(19) United States
(12) Patent Application Publication
(10) Pub. No.: US 2026/0364213 A1
(43) Pub. Date: Nov. 19, 2026

(54) RADIOLABELED COMPOUNDS OF TRIMETHOPRIM AND USES THEREOF

(71) Applicant: THE TRUSTEES OF THE UNIVERSITY OF PENNSYLVANIA, Philadelphia, PA (US)

(72) Inventors: Mark A. SELLMYER, Philadelphia, PA (US); Nitika SHARMA, Philadelphia, PA (US); Jean M. ETERSQUE, Philadelphia, PA (US); Swarbhanu SARKAR, Philadelphia, PA (US); Hwan LEE, Philadelphia, PA (US)

(21) Appl. No.: 19/488,140

(22) PCT Filed: May 30, 2024
(86) PCT No.: PCT/US2024/031640

§ 371 Date: Nov. 26, 2025

Related U.S. Application Data
(60) Provisional application No. 63/505,029, filed on May 30, 2023.

(51) Int. Cl. A61K 51/04; A61P 35/00; C07B 59/00; C07D 239/49; C12N 9/06; C12N 15/62

(52) U.S. Cl. CPC A61K 51/0459; A61P 35/00; C07B 59/002; C07D 239/49; C12N 9/003; C12N 15/62; A61K 2121/00; C07B 2200/05; C07K 2319/10; C12Y 105/01003

(57) ABSTRACT

The present disclosure provides compounds of formula I, II, or III or pharmaceutically acceptable salts thereof, wherein R¹-R⁴, R¹¹-R¹⁴, R³¹, R³³, and R³⁴ are defined herein. Also provided are compositions comprising the compounds, methods of using the compounds for delivering radiation to dihydrofolate reductase (DHFR) expressing cells or tissues, killing a cell or tissue comprising E. coli dihydrofolate reductase (eDHFR), or treating a cancer in a subject in need thereof.

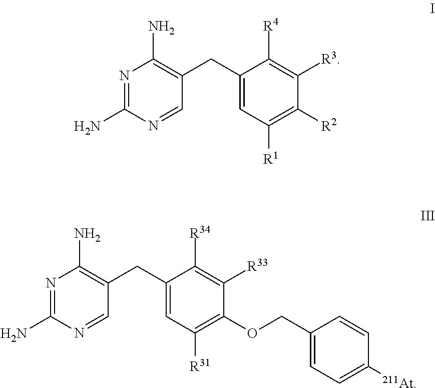

### CROSS-REFERENCE TO RELATED APPLICATIONS

This application claims the benefit of the priority of U.S. Provisional Patent Application No. 63/505,029, filed May 30, 2023, the contents of which are incorporated by reference herein.

### TECHNICAL FIELD

This disclosure relates to the radiolabeled compounds of trimethoprim (TMP) and methods of use thereof.

### BACKGROUND

External-beam radiotherapy (EBRT), the most common form of radiation therapy (RT), has been a staple treatment for solid tumors. EBRT can directly kill cancer cells and simultaneously affect the tumor microenvironment (TME). It is a standard-of-care treatment in some localized tumors and is also used for palliation in advanced cancers. However, key limitations of EBRT are its inability to effectively treat widely metastatic tumors and significant side effects to normal tissues that are in the treatment field.

Radioligand therapeutics (RLT) that are administered intravenously have the ability to target multifocal diseases in the human body. One approach is to use gene and cell therapies to deliver exogenous (synthetic) targets to multifocal diseases and then use RPT to administer therapeutic radiation to any sites that received the exogenous target.

Adoptive cell therapies such as chimeric antigen receptor (CAR)-T therapy have demonstrated promising results in various blood cancers and lymphoma. However, multiple challenges have hindered the application to a broader set of solid tumors. Despite its success in the hematological malignancies, CAR-T therapy faces limitations in treating solid tumors and can lead to life-threatening immune-related conditions like cytokine release syndrome, neurotoxicity, and on-target-off target effects. While suicide switches could alleviate the toxicity associated with CART cell therapy, they suffer from limitations such as the spatial and temporal control, potential immunogenic responses, and lack useful approaches for quantitative in vivo and clinical imaging. RLT after CAR T cells have localized to a tumor could provide a kill switch to alleviate a CAR T cell derived toxicity or to deliver therapeutic radiation to the surrounding tissue.

What is needed is more effective and selective therapies for treating cancer, such as solid tumors.

### SUMMARY

In some embodiments, the disclosure provides compounds of formula I or pharmaceutically acceptable salts thereof, wherein R¹-R⁴ are defined herein:

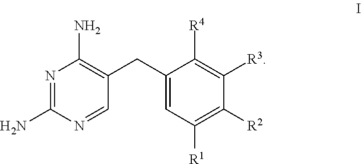

In some embodiments, the disclosure provides compounds of formula II or pharmaceutically acceptable salts thereof, wherein R¹¹-R¹⁴ are defined herein:

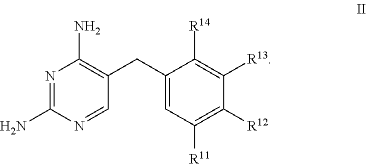

In further embodiments, the disclosure provides compounds of formula III or pharmaceutically acceptable salts thereof, wherein R³¹, R³³, and R³⁴ are defined herein:

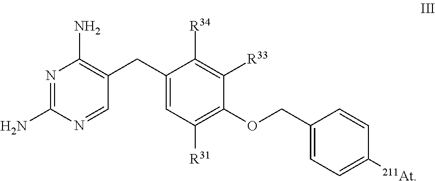

In further embodiments, the disclosure provides compositions comprising one or more compounds described herein and a pharmaceutically acceptable carrier or diluent.

In yet other embodiments, the disclosure provides methods of delivering radiation to dihydrofolate reductase (DHFR) expressing cells or tissues in a subject, comprising administering a compound described herein to the DHFR expressing cells or tissues.

In still further embodiments, the disclosure provides methods of killing a cell or tissue comprising E. coli dihydrofolate reductase (eDHFR), comprising exposing the cell or tissue to a compound described herein.

In other embodiments, the disclosure provides methods of treating a cancer in a subject in need thereof, comprising (a) delivering eDHFR to cells or tissue surrounding the cancer; and (b) administering an effective amount of a compound described herein to said subject.

Other aspects and embodiments of the invention will be readily apparent from the following detailed description of the invention.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

FIGS. 1A and 1B is a schematic of genetic constructs of eDHFR-YFP-T2A-Luciferase (eDHFR-YFP) and eDHFR-Luciferase-T2A-mCherry (eDHFR-Luc) construct, YFP and Renilla Luciferase are directly fused to the C-terminus of eDHFR, respectively. FIG. 1C is a schematic of eDHFR_Flag construct FIG. 1D is a bar graph showing uptake in HEK cells. eDHFR+ and wild type cells receive 0.037 MBq of [¹²⁵I]mI-TMP, incubated for 60 min at 37 °C, for blocked controls, preincubation with 10 μm of TMP. FIG. 1E is a bar graph showing uptake in OVCAR8_eDHFR_Luc and WT cells incubated with 0.037 MBq of [¹³¹I]mI-TMP. FIG. 1F is a bar graph showing uptake of I45_eDHFR_flag and WT cells with 0.037 MBq of [¹²⁵I]mI-TMP incubated for 60 min at 37 °C. Uptake was assayed on gamma counter. n=3, data points are mean±SD.

FIGS. 2A-2C are bar graphs showing the viability of OVCAR 8-eDHFR cells assessed after dosing them 1.85-37 MBq of [¹³¹I]mI-TMP, at different incubation times. In FIGS. 2A and 2B, cells were incubated with 1.85 MBq and 18.5 MBq of [¹³¹I]mI-TMP for 60 min, respectively. Cell viability was measured on days 1, 3, 7 and 12 using MTT reagents and absorbance was measured via plate reader at 590 nm. In FIG. 2C, cells were incubated with 37 MBq of [¹³¹I]mI-TMP for 20 min. Cell viability was measured on days 1, 3, 7 and 12 using MTT reagents and absorbance was measured via plate reader at 590 nm.

FIGS. 3A and 3B are bar graphs showing cell viability of HCT116 cells after 5b treatment. In FIG. 3A, eDHFR+ and WT HCT116 cells were dosed with 3.7 MBq of [¹³¹I]mI-TMP at 37 °C, for 2 h incubation, Cell viability was measured on days 1, 3, 7 and 10 using MTT reagents and absorbance was measured via plate reader at 590 nm. FIG. 3B is the same as 3A, but with 4 h incubation. n=3, data points are mean±SD. Thyroid uptake was blocked with a pre-treatment of SSKI.

FIG. 4 is a bar graph showing ex vivo biodistribution quantification at 1-hour post 0.67 MBq of [¹³¹I]mI-TMP injection via tail vein in healthy Balb/c mice. Total radioactive uptake per organ, in counts per minute (CPM), was determined by γ-counter (Wizard Detector, Perkin Elmer). No SSKI was administered.

FIG. 5A is a bar graph showing ex vivo biodistribution quantification at 3-hours post 1.85 MBq of [¹³¹I]mI-TMP injection for major organs in a HCT116 eDHFR-positive (left bar in each group) and negative tumor-bearing athymic nude mice (right bar in each group). Total radioactive uptake per organ, in CPM, was determined by γ-counter (Wizard Detector, Perkin Elmer). FIG. 5B is a bar graph showing [¹³¹I]I-TMP distribution in eDHFR+(left bar) and WT tumor (right bar). Unpaired t test; **p=0.0037.

FIGS. 6A and 6B illustrate [¹³¹I]mI-TMP uptake in CAR T cells. FIG. 6A is a schematic of the Fibroblast Activation Protein (FAP) CAR construct is shown. eDHFR protein is fused directly to the C-terminus of the CD3zeta domain of the FAP CAR. FIG. 6B is a bar graph of the uptake in FAP_CAR_eDHFR_DF IRES BFP cells compare to Non-transduced cells. Both cell populations received 0.074 MBq of [¹³¹I]mI-TMP, incubated for 60 min at 37 °C.

FIG. 7A is a radio HPLC profile of the isolated [¹³¹I]mI-TMP. FIG. 7B is the corresponding 254 nm UV chromatogram

FIGS. 8A-8C show serum stability data. FIG. 8A is the LC-MS profile of non-radioactive 3a. FIG. 8B is the LC-MS profile of non-radioactive 3a after incubating with FBS at 37 °C for 72 h. FIG. 8C is a ling graph of the serum stability of [¹³¹I]mI-TMP. 0.925 MBq of [¹³¹I]mI-TMP was incubated with FBS or PBS (pH=7.4) at 37 °C and stability was monitored up to 72 h by TLC (iTLC/saline)

FIG. 9 is a differential scanning fluorimetry (DSF) spectrum. This figure shows comparison of binding to TMP and mI-TMP (not radiolabeled) to E. coli DHFR. Purified WT eDHFR (10 mg/mL stock); mI-TMP (10 mM stock); TMP (10 mM stock); NADPH. mI-TMP (not radiolabeled) stabilizes eDHFR as TMP.

FIG. 10A is a bar graph for uptake studies in HCT116 cells. eDHFR+ and WT HCT116 cells were incubated with 0.037 MBq of [¹²⁵I]mI-TMP for 60 min at 37 °C, for blocked controls the cells were preincubated with 10 μM TMP. FIG. 10B is a line graph of a washout experiment to evaluate the retention of [¹³¹I]mI-TMP in HCT116 cells. Following 20 min uptake, the cells were washed thrice with 1% DMSO/PBS and were sampled serially at 1 h, 2 h and 4 h. Uptake was assayed on gamma counter. n=3, ata points are mean±SD.

FIG. 11 is a bar graph showing ex vivo biodistribution quantification at 1-(left bar in each group), 4- (middle bar in each group), and 24-hours (right bar in each group) post [¹³¹I]mI-TMP injection via tail vein in healthy Balb/c mice. See, Example 3.

FIG. 12 is a bar graph showing tumor size for mice were given 18.5 MBq of [¹³¹I]mI-TMP via tail vein. Tumor size was monitored by caliper over 15 days. See, Example 2.

FIGS. 13A and 13B are bar graphs showing [¹³¹I]mI-TMP uptake in FAP_CAR_eDHFR cells with 0.074 MBq (13B) and washout studies (13A) after 5 h.

FIGS. 13C and 13D are graphs showing eDHFR expression in FAP_CAR_eDHFR_(df) and FAP_CAR_eDHFR_(df)_BFP, respectively. FIG. 13E is a bar graph showing the results when the uptake experiment of FIG. 13C was repeated using CART cells at day 5 of expansion and showing smaller differences specific uptake. FIG. 13F and FIG. 13G are fibroblast activation protein (FAP) CAR constructs. eDHFR protein is fused directly to the C-terminus of the CD3zeta domain of the FAP CAR.

FIG. 14 is a bar graph for the hepatocyte spheroid assay.

FIG. 15 is are dose response curves for Example 4.

FIG. 16A-16C are dose-response curves for Example 5. FIG. 16A is for ²¹¹At-PAB-TMP treatment in IMR5 cells expressing eDHFR-YFP localized to the DNA, nucleus, and cytoplasm. Higher cytotoxicity was seen with increasing proximity to DNA, resulting in significantly lower EC₅₀ values. FIG. 16B is a bar graph of the EC50s for ²¹¹At-PAB-TMP in different eDHFR cell lines. (**P<0.01, ****P<0.0001). FIG. 16C is a curve showing ²¹¹At-PTT in eDHFR IMR5 cells (control for no difference in cellular toxicity of an alpha therapy in each eDHFR expressing cell line).

FIG. 17 is a model showing the differences in distance for targeting alpha therapies at the nucleus, cytoplasm, and cell membrane.

FIG. 18 are radio-HPLC spectra showing the in vitro stability of [²¹¹At]At-TMP. The traces show stability of [²¹¹At]TMP in aqueous media for up to 6 hours.

FIG. 19 are line graphs of the measurement of the dissociation constant (K_{d}) of [¹²⁵I]I-TMP binding to eDHFR-YFP under ligand-depleting conditions. Linear best-fit lines were drawn across the eDHFR-YFP subtypes in I45 (R²=0.98) and SKOV3 (R²=0.92) cells. The K_{d} (y-intercept/slope) was 0.65±0.11 nM in I45 and 0.13±0.17 nM in SKOV3 cells (mean±SEM).

FIGS. 20 and 21 are curves showing the cytotoxicity of doxorubicin and TMP. FIG. 20: In both I45 and SKOV3 cell lines, wild-type and eDHFR-YFP-expressing cells showed comparable cytotoxicity from doxorubicin. FIG. 21: I45 and SKOV3 cells expressing the membrane-bound eDHFR-YFP showed higher cytotoxicity from non-radiolabeled TMP.

FIG. 22 are bar graphs showing the intracellular distribution of eDHFR-YFP subtypes. The subcellular fractions were used to isolate the cytotoxicity of treating eDHFR-YFP only in the desired subcellular compartments, as per Supplemental Methods 2.

FIG. 23 is a diagram of the “incident angle” effect, where membrane-associated ²¹¹At decay imparts more damage to the plasma membrane. In this figure, Let r, t, 0, and L be defined as shown on the figure. Using the Pythagorean theorem, L can be expressed as

L=\sqrt{(d cos θ)²−d²+(r+t)²}\sqrt{−(d cos θ)²−d²+r²}
where d is the distance from the alpha emitter to the center of the cell.
The ratio of average length within the plasma membrane traversed by an alpha particle from a 6 μm radius nucleus (d=4.5 μm) vs. plasma membrane (d=r) is:

$${\int }_{0 °}^{360 °} {L}_{nucleus} d θ / 2 π : {\int }_{0 °}^{360 °} {L}_{membrane} d θ / 2 π$$

For r=12 μm and t=8 nm, a 3.2-fold more damage to the plasma membrane from a membrane-associated alpha decay compared to an alpha decay in the nucleus was obtained.
Compared to a cytoplasmic alpha emitter with d=10 μm, 2.5-fold more damage from a membrane-associated alpha emitter was obtained.

FIG. 24 is a three-dimensional model of [²¹¹At]At-TMP bound to eDHFR, fused to the Sso7d DNA-binding domain at the N-terminus. The distance from the ²¹¹At atom (shaded sphere) to the adjacent DNA strand was estimated to be 5 nm (dashed line).

FIG. 25 is an illustration of the subcellular eDHFR & [²¹¹At]At-TMP system and chemical approach. eDHFR-YFP fusion protein was localized to the DNA, nucleus, cytoplasm, and cell membrane for subsequent targeting with [²¹¹At]At-TMP in human cancer cells.

FIGS. 26A-26D show the characterization of subcellular eDHFR-YFP expression in human cancer cells. FIG. 26A are live-cell confocal microscopy images of I45 and SKOV3 cells expressing eDHFR-YFP in specific subcellular locations. FIG. 26B are fluorescence images showing the co-localization in the cell nucleus between DNA (Hoechst 33342) and eDHFR (YFP). FIG. 26C are plots of the distance from the cell nucleus to cytoplasmic eDHFR-YFP and membrane-bound eDHFR-YFP, expressed as probability histograms. **P<0.01, ***P<0.001. FIG. 26D are flow cytometry histograms of I45 and SKOV3 cells expressing eDHFR-YFP in specific subcellular locations.

FIGS. 27A-27C show the pharmacologic characterization of radiolabeled TMP-eDHFR binding. FIG. 27A are the representative saturation binding curves for [¹²⁵I]I-TMP in I45 and SKOV3 cells. FIG. 27B are plots showing the strongly positive correlation between Bₘₐₓ and YFP fluorescence intensity in I45 (R²=0.97) and SKOV3 (R²=0.85) cell lines. FIG. 27C are δ⁻¹ vs. [R_{T}]⁻¹ plot to determine the dissociation constant (K_{d}) of [²¹¹At]At-TMP as the slope of the linear best-fit line.

FIGS. 28A-28F show the cytotoxicity of [²¹¹At]At-TMP based on subcellular targeting. FIG. 28A are plots of eDHFR-YFP-expressing I45 and SKOV3 cells showed significantly higher cytotoxicity from [²¹¹At]At-TMP compared to the wild-type cells. FIGS. 28B and 27C are plots of the cytotoxicity of [²¹¹At]At-TMP+10 μM cold TMP (FIG. 28B) and free ²¹¹At (FIG. 28C) that was comparable across the wild-type and eDHFR-YFP-expressing cells in both I45 and SKOV3. FIG. 28D are plots where the [²¹¹At]At-TMP concentration was converted to the number of bound [²¹¹At]At-TMP decays/cell. FIG. 28E are bar graphs where EC₅₀ values were plotted, with horizontal bars representing the isolated EC₅₀ of eDHFR-YFP only in the desired subcellular compartments. FIG. 28F are bar graphs comparing between the relative cytotoxicity and relative nuclear dose to calculate the RBE for eDHFR-YFP in different subcellular compartments, with reference to cytoplasmic eDHFR-YFP. *P<0.05, **P<0.01, ***P<0.001, ****P<0.0001.

FIG. 29A-29C show the three-dimensional dosimetry of scTAT in microscopic tumor cell clusters. FIG. 29A is a plot of equivalent alpha dose per decay from self- and cross-dose contributions. FIG. 28B is a plot to relative cytotoxicity of DNA-bound, nuclear, and membrane-bound TAT per decay, with reference to cytoplasmic TAT. Cytotoxic advantage of DNA and nuclear targeting is maximized below the threshold tumor size of 100 μm. FIG. 29C is a diagram of a spectrum of tumor cell cluster sizes with pathologic (top boxes) and hematologic (bottom boxes) definitions. Individual circulating tumor cells (CTCs) and most CTC clusters are smaller than 100 μm in size, subject to high cytotoxicity from DNA-bound and nuclear TAT.

FIGS. 30A and 30B illustrate in vivo [¹³¹I]I-TMP uptake in I45 mesothelioma tumors. FIG. 30A is a bar graph of a limited biodistribution that was performed at 1, 3, and 24 hours after injection of 18.5 MBq [¹³¹I]I-TMP. FIG. 30B is a bar graph of the ratio of uptake in eDHFR tumors compared to WT tumors is plotted over time. Individual mouse ratios are shown. In these figures, the left bar in each group is at 1 hour, the middle bar in each group is at 3 hours, and the right bar in each group is at 24 hours.

FIGS. 31A and 31B are plots showing that [¹³¹I]I-TMP shows in vivo cytotoxicity toward eDHFR-expressing I45 cells. FIG. 31A is a plot where I45 WT and eDHFR cells were implanted and grown in CD1 nude mice for −2 weeks prior to administration of a single dose of 18.5 MBq [¹³¹I]I-TMP on day 9 of initial tumor inoculation. The day of introduction of [¹³¹I]I-TMP or saline in mice is considered as day 0 in the above diagram. Tumor volumes were measured using calipers. Error bars represent the standard deviation. At each time point, the first set of dots correspond to I45 saline, second set of dots correspond to I45 WT [¹³¹I]I-TMP, the third set of dots correspond to I45 eDHFR-FLAG saline, and the fourth set of dots correspond to I45 eDHFR-FLAG [¹³¹I]I-TMP. FIG. 31B is a plot where Tumor growth is expressed on the Y-axis as a fold change over baseline and averaged (n=7).

FIGS. 32A and 32B are tumor growth curves after [²¹¹At]At-TMP treatment. For FIG. 32A, wild type (WT) I45 cells, Cytoplasmic eDHFR I45, and Nuclear eDHFR I45 cells were injected into nude mice approximately 3 weeks prior to [²¹¹At]At-TMP treatment. Approximately 30 uCi was injected into all groups via tail vein injection on days 0, 3, and 6 after SSKI injection. Tumor growth was monitored by caliper measurements. For FIG. 32B, relative tumor volume as compared to the day 0 tumor volume. WT vs. cytoplasmic p-value is 0.009 (**) for absolute volume and 0.02 (*) for relative volume.

FIG. 33 are the LCMS spectra of reaction mixture of Method B2.

### DETAILED DESCRIPTION OF ILLUSTRATIVE EMBODIMENTS

Traditional therapeutic agents for treating cancer rely on the target density of cancer cells for delivery of the cytotoxic agent. However, eDHFR can be delivered into tissues by a variety of genetic mechanisms. Thus, since the compounds of the disclosure target DHFR, they are selective and specific when it comes to killing eDHFR cells or cells near eDHFR expressing cells such as neoplastic cells.

Escherichia coli dihydrofolate reductase (eDHFR) is a biologically orthogonal target. Human cells can be engineered to express eDHFR using vectors. eDHFR also can contain a localization domain, such as the nuclear localization sequence (NLS), thus permitting attack of the nucleus or other subcellular organelles. As used herein, eDHFR refers to a bacterial protein, that can be engineered into human and mammalian cells, which allows for high contrast (e.g., target to background) and high therapeutic index between eDHFR expressing tissues and normal mammalian cells.

The compounds described herein are radiotherapeutics and are useful in radioligand therapy (RLT). The compounds permit delivery of alpha (such as alpha particles emitted by ²¹¹At) or beta (such as beta particles emitted by ¹³¹I) particles to cells, for focused killing of such cells. The use of alpha particles has several advantages for cancer treatment. Alpha particles interact with the cell nucleus and can interact with DNA by traversing the cytoplasm. For example, alpha particles have the capability of depositing dense energy over a short path length to efficiently cause lethal double-strand DNA breaks. They then generate reactive oxygen species to damage the target cell. Alpha particles also produce nanometer-range recoil radiation, which contributes to the cytotoxicity when the radionuclide is located close to DNA. Further, linear energy transfer (LET) of alpha particles can change during its path to the target cells, thereby resulting in a maximum biological effect once it reaches the target cell. Alpha radiation also can cause direct DNA damage independent of oxygen level, unaffected by hypoxia-induced radiation resistance. Alpha particles also are less sensitive to cell diameters, traveling across multiple cell diameters to cause damage to nearby tumor cells with low target expression, while simultaneously avoiding toxicity to surrounding normal tissues. Beta particles, however, can interact and treat surrounding cells and tissues, thereby providing another layer of treatment.

### DEFINITIONS

In the present disclosure the singular forms “a,” “an” and “the” include the plural reference, and reference to a particular numerical value includes at least that particular value, unless the context clearly indicates otherwise. Thus, e.g., a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially.” In other cases, the gradations used in a series of values may be used to determine the intended range available to “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

As used herein, “treating” encompasses treating a subject clinically diagnosed as having a disease or medical condition. In one embodiment, the subject is treated and the disease or medical condition is eradicated, i.e., the subject is cured.

“Patient” or “subject” as used herein refer to a mammalian animal. In one embodiment, the patient or subject is a human. In another embodiment, the patient or subject is a veterinary or farm animal, a domestic animal or pet, or animal normally used for clinical research. In still a further embodiment, the subject or patient has cancer. The subject or patient has either been recognized as having or at risk of having cancer.

“Imaging” as used herein refers to any method of scanning the body of a subject using techniques such as positron emission tomography (PET) or single photon emission computed tomography (SPECT), among others.

The term “alkyl” is used herein to refer to both straight- and branched-chain saturated aliphatic hydrocarbon groups. In some embodiments, an alkyl group has 1 to about 10 carbon atoms (C₁₋₁₀). In other embodiments, an alkyl group has 1 to about 6 carbon atoms (C₁₋₆). In further embodiments, an alkyl group has 1 to about 4 carbon atoms (C₁₋₄). In yet other embodiments, the alkyl is methyl. In still further embodiments, the alkyl is ethyl. In other embodiments, the alkyl is propyl. In further embodiments, the alkyl is butyl. In yet other embodiments, the alkyl is pentyl. In further embodiments, the alkyl is hexyl. An alkyl is optionally substituted with one, two, or three substituents that are halo (F, Cl, Br, or I), C₁₋₆alkyl, OH, C₁₋₆alkoxy, C₃₋₈cycloalkyl, or aryl.

The term “alkoxy” as used herein refers to the O-(alkyl) group, where the point of attachment is through the oxygen-atom and the alkyl group is defined above. In some embodiments, the alkoxy is methoxy. In other embodiments, the alkoxy is ethoxy. In further embodiments, the alkoxy is propoxy. In yet other embodiments, the alkoxy is butoxy. In still further embodiments, the alkoxy is pentoxy. In other embodiments, the alkoxy is hexoxy. An alkoxy is optionally substituted with one, two, or three substituents that are halo (F, Cl, Br, or I), C₁₋₆alkyl, OH, C₁₋₆alkoxy, C₃₋₈cycloalkyl, or aryl.

The term “cycloalkyl” is used herein to refer to a cyclic, saturated hydrocarbon group. In some embodiments, a cycloalkyl has 3 to about 10 carbon atoms (C₃₋₁₀). In other embodiments, a cycloalkyl has 3 to about 8 carbon atoms (C₃₋₈). In further embodiments, a cycloalkyl has 3 to about 6 carbon atoms (C₃₋₆). In yet other embodiments, the cycloalkyl is cyclopropyl. In further embodiments, the alkyl is cyclobutyl. In yet other embodiments, the alkyl is cyclopentyl. In further embodiments, the alkyl is cyclohexyl. A cycloalkyl is optionally substituted with one, two, or three substituents that are halo (F, Cl, Br, or I), C₁₋₆alkyl, OH, C₁₋₆alkoxy, C₃₋₈cycloalkyl, or aryl.

The term “aryl” refers to carbocyclic aromatic groups having from 6 to 10 carbon atoms (“C₆₋₁₀”) such as phenyl, naphthyl, and the like. An aryl is optionally substituted with one, two, or three substituents that are halo (F, Cl, Br, or I), C₁₋₆alkyl, OH, C₁₋₆alkoxy, C₃₋₈cycloalkyl, or aryl.

The term “radiolabeled halo” refers to ¹⁸F, ¹²³I, ¹²⁵I, ¹²⁴I, ¹³¹I, ³²Cl, ³³Cl, ³⁴Cl, ⁷⁴Br, ⁷⁵Br, ⁷⁶Br, ⁷⁷Br, ⁷⁸Br, or ²¹¹At. In some embodiments, the radiolabeled halo is ¹⁸F. In other embodiments, the radiolabeled halo is ¹²³I. In further embodiments, the radiolabeled halo is ¹²⁵I. In yet other embodiments, the radiolabeled halo is ¹²⁴I. In still further embodiments, the radiolabeled halo is ¹³¹I. In other embodiments, the radiolabeled halo is ³²Cl. In further embodiments, the radiolabeled halo is ³³Cl. In yet other embodiments, the radiolabeled halo is ³⁴Cl. In still further embodiments, the radiolabeled halo is ⁷⁴Br. In other embodiments, the radiolabeled halo is ⁷⁵Br. In further embodiments, the radiolabeled halo is ⁷⁶Br. In still other embodiments, the radiolabeled halo is ⁷⁷Br. In yet further embodiments, the radiolabeled halo is ⁷⁸Br. In other embodiments, the radiolabeled halo is ²¹¹At.

The term “TMP” refers to trimethoprim, having the following structure:

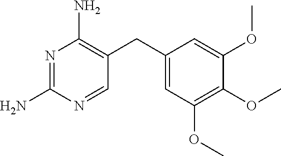 [SMILES: COc1cc(Cc2cnc(N)nc2N)cc(OC)c1OC]

### The Compounds

The present disclosure provides compounds of formula I, II, and III.

### A. Compounds of Formula I

In certain aspects, the disclosure provides compounds of formula I or pharmaceutically acceptable salts thereof.

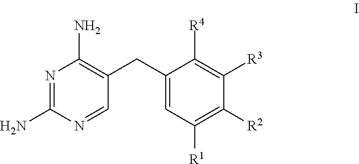

According to the disclosure, R¹ is H or radioactive halo. In some embodiments, R¹ is H. In other embodiments, R¹ is a radioactive halo such as ¹⁸F, ³²Cl, ³³Cl, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I or ²¹¹At. In further embodiments, R¹ is ¹⁸F. In yet other embodiments, R¹ is ³²Cl. In still further embodiments, R¹ is ³³Cl. In other embodiments, R¹ is ³⁴Cl. In some embodiments, R¹ is ¹²⁵I. In other embodiments, R¹ is ¹³¹I. In further embodiments, R¹ is ²¹¹At. In other embodiments, R¹ is ⁷⁴Br. In yet other embodiments, R¹ is ⁷⁵Br. In still further embodiments, R¹ is ⁷⁶Br. In other embodiments, R¹ is ⁷⁷Br. In further embodiments, R¹ is ⁷⁸Br.

According to the disclosure, R² is H, halo, C₁₋₆alkoxy, C₁₋₆alkyl, or aryl. In some embodiments, R² is H. In further embodiments, R² is halo (not radiolabeled) such as F, Cl, Br, or I. In other embodiments, R² is C₁₋₆alkoxy, such as methoxy, ethoxy, propoxy, butoxy, pentoxy or hexoxy, or such as methoxy. In further embodiments, R² is C₁₋₆alkyl, such as methyl, ethyl, propyl, butyl, pentyl, or hexyl, or such as methyl. In yet other embodiments, R² is aryl such as phenyl.

According to the disclosure, R³ is H or radioactive halo. In some embodiments, R³ is H. In other embodiments, R³ is a radioactive halo such as ¹⁸F, ³²Cl, ³³Cl, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I or ²¹¹At. In further embodiments, R³ is ¹⁸F. In yet other embodiments, R³ is ³²Cl. In still further embodiments, R³ is ³³Cl. In other embodiments, R³ is ³⁴Cl. In some embodiments, R³ is ¹²⁵I. In other embodiments, R³ is ¹³¹I. In further embodiments, R³ is ²¹¹At. In other embodiments, R³ is ⁷⁴Br. In yet other embodiments, R³ is ⁷⁵Br. In still further embodiments, R³ is ⁷⁶Br. In other embodiments, R³ is ⁷⁷Br. In further embodiments, R³ is ⁷⁸Br.

According to the disclosure, R⁴ is H or radioactive halo. In some embodiments, R⁴ is H. In other embodiments, R⁴ is a radioactive halo such as ¹⁸F, ³²Cl, ³³Cl, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I or ²¹¹At. In further embodiments, R⁴ is ¹⁸F. In yet other embodiments, R⁴ is ³²Cl. In still further embodiments, R⁴ is ³³Cl. In other embodiments, R⁴ is ³⁴Cl. In some embodiments, R⁴ is ¹²⁵I. In other embodiments, R⁴ is ¹³¹I. In further embodiments, R⁴ is ²¹¹At. In other embodiments, R⁴ is ⁷⁴Br. In yet other embodiments, R⁴ is ⁷⁵Br. In still further embodiments, R⁴ is ⁷⁶Br. In other embodiments, R⁴ is ⁷⁷Br. In further embodiments, R⁴ is ⁷⁸Br.

According to the disclosure, at least one of R¹, R³, and R⁴ is a radioactive halo in the compound of formula I.

In certain embodiments, the compound is of formula I-A:

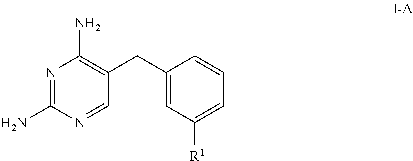

In other embodiments, the compound is of formula I-B:

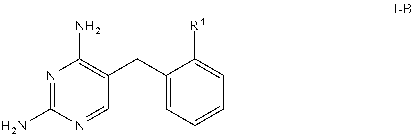

In some embodiments, the compound is

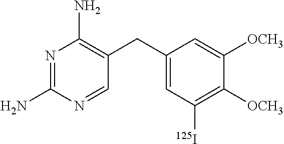 [SMILES: COc1cc(Cc2cnc(N)nc2N)cc([125I])c1OC]

([¹²⁵I]mI-TMP) or a pharmaceutically acceptable salt thereof. In other embodiments, the compound is

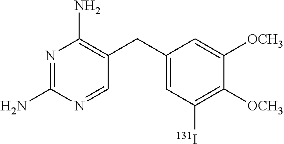 [SMILES: COc1cc(Cc2cnc(N)nc2N)cc([131I])c1OC]

([¹³¹I]mI-TMP) or a pharmaceutically acceptable salt thereof. In further embodiments, the compound is

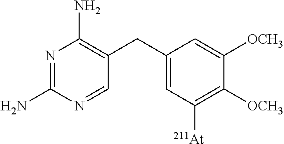

(²¹¹At-mAt-TMP) or a pharmaceutically acceptable salt thereof. In yet other embodiments, the compound is

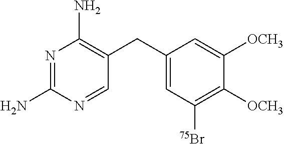 [SMILES: COc1cc(Cc2cnc(N)nc2N)cc([75Br])c1OC]

or a pharmaceutically acceptable salt thereof. In still further embodiments, the compound is

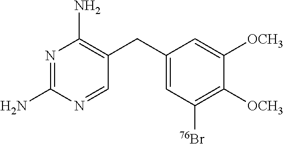 [SMILES: COc1cc(Cc2cnc(N)nc2N)cc([76Br])c1OC]

or a pharmaceutically acceptable salt thereof. In other embodiments, the compound is

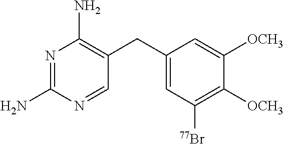 [SMILES: COc1cc(Cc2cnc(N)nc2N)cc([77Br])c1OC]

or a pharmaceutically acceptable salt thereof.

The inventors found that the compounds of formula I, having a radiolabel at the meta position were more easily synthesized via precursors, including Sn precursors, and had increased binding affinity and cell uptake, as compared to compounds similarly labeled at the para position.

### B. Compounds of Formula II

In other aspects, the disclosure provides compounds of formula II or pharmaceutically acceptable salts thereof.

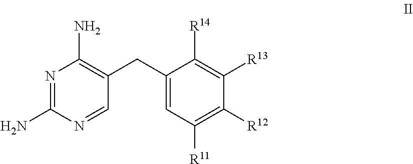

According to the disclosure, R¹¹ is H, C₁₋₆alkoxy, C₁₋₆alkyl, or aryl. In some embodiments, R¹¹ is H. In other embodiments, R¹¹ is C₁₋₆alkoxy, such as methoxy, ethoxy, propoxy, butoxy, pentoxy or hexoxy, or such as methoxy. In further embodiments, R¹¹ is C₁₋₆alkyl, such as methyl, ethyl, propyl, butyl, pentyl, or hexyl, or such as methyl. In yet other embodiments, R¹¹ is aryl such as phenyl.

According to the disclosure, R¹² is a radioactive halo. In some embodiments, R¹² is ¹⁸F, ³²Cl, ³³Cl, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I, or ²¹¹At. In other embodiments, R¹² is ¹²⁵I. In yet other embodiments, R¹² is ¹³¹I. In further embodiments, R¹² is ²¹¹At. In yet other embodiments, R¹² is ⁷⁵Br. In still further embodiments, R¹² is ⁷⁶Br. In other embodiments, R¹² is ⁷⁷Br. In further embodiments, R¹² is ¹⁸F. In yet other embodiments, R¹² is ³²Cl. In still further embodiments, R¹² is ³³Cl. In other embodiments, R¹² is ³⁴Cl. In further embodiments, R¹² is ⁷⁴Br. In still other embodiments, R¹² is ⁷⁸Br. In yet further embodiments, R¹² is ¹²³I. In other embodiments, R¹² is ¹²⁴I.

According to the disclosure, R¹³ is H, C₁₋₆alkoxy, C₁₋₆alkyl, or aryl. In some embodiments, R¹³ is H. In other embodiments, R¹³ is C₁₋₆alkoxy, such as methoxy, ethoxy, propoxy, butoxy, pentoxy or hexoxy, or such as methoxy. In further embodiments, R¹³ is C₁₋₆alkyl, such as methyl, ethyl, propyl, butyl, pentyl, or hexyl, or such as methyl. In yet other embodiments, R¹³ is aryl such as phenyl.

According to the disclosure, R¹⁴ is H, C₁₋₆alkoxy, C₁₋₆alkyl, or aryl. In some embodiments, R¹⁴ is H. In other embodiments, R¹⁴ is C₁₋₆alkoxy, such as methoxy, ethoxy, propoxy, butoxy, pentoxy or hexoxy, or such as methoxy. In further embodiments, R¹⁴ is C₁₋₆alkyl, such as methyl, ethyl, propyl, butyl, pentyl, or hexyl, or such as methyl. In yet other embodiments, R¹⁴ is aryl such as phenyl.

According to the disclosure, at least one of R¹¹, R¹³ and R¹⁴ is C₁₋₆alkyl or aryl in the compounds of formula II.

### D. Compounds of Formula III

In further aspects, the disclosure provides compounds of formula III:

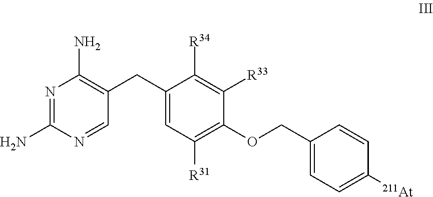

According to the disclosure, R³¹ is H, C₁₋₆alkoxy, C₁₋₆alkyl, or aryl. In some embodiments, R³¹ is H. In other embodiments, R³¹ is C₁₋₆alkoxy, such as methoxy, ethoxy, propoxy, butoxy, pentoxy or hexoxy, or such as methoxy. In further embodiments, R³¹ is C₁₋₆alkyl, such as methyl, ethyl, propyl, butyl, pentyl, or hexyl, or such as methyl. In yet other embodiments, R³¹ is aryl such as phenyl.

According to the disclosure, R³³ is H, C₁₋₆alkoxy, C₁₋₆alkyl, aryl, ¹²⁵I, ¹³¹I ⁷⁵Br, ⁷⁶Br, ⁷⁷Br, or ²¹¹At. In some embodiments, R³³ is H. In other embodiments, R³³ is C₁₋₆alkoxy, such as methoxy, ethoxy, propoxy, butoxy, pentoxy or hexoxy, or such as methoxy. In further embodiments, R³³ is C₁₋₆alkyl, such as methyl, ethyl, propyl, butyl, pentyl, or hexyl, or such as methyl. In yet other embodiments, R³³ is aryl such as phenyl.

According to the disclosure, R³⁴ is H, C₁₋₆alkoxy, C₁₋₆alkyl, aryl, ¹²⁵I, ¹³¹I ⁷⁵Br, ⁷⁶Br, ⁷⁷Br, or ²¹¹At. In some embodiments, R³⁴ is H. In other embodiments, R³⁴ is C₁₋₆alkoxy, such as methoxy, ethoxy, propoxy, butoxy, pentoxy or hexoxy, or such as methoxy. In further embodiments, R³⁴ is C₁₋₆alkyl, such as methyl, ethyl, propyl, butyl, pentyl, or hexyl, or such as methyl. In yet other embodiments, R³⁴ is aryl such as phenyl.

In some embodiments, R³² and R³⁴ are C₁₋₆alkoxy. In further embodiments, R³² and R³⁴ are methoxy.

In other embodiments, the compound of formula III is

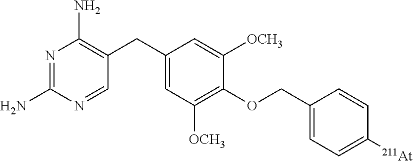

or a pharmaceutically acceptable salt thereof.

In further embodiments, the disclosure provides the following compound:

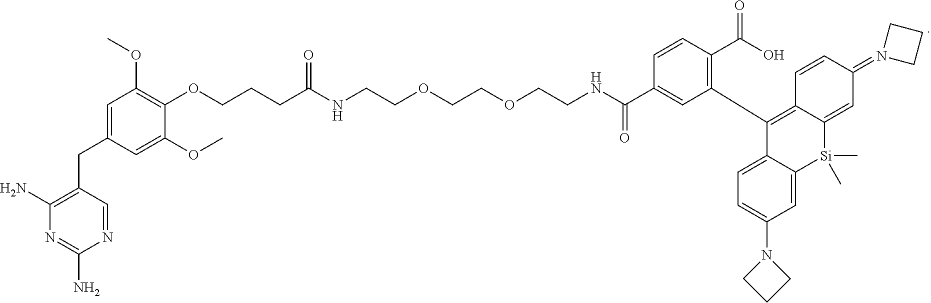

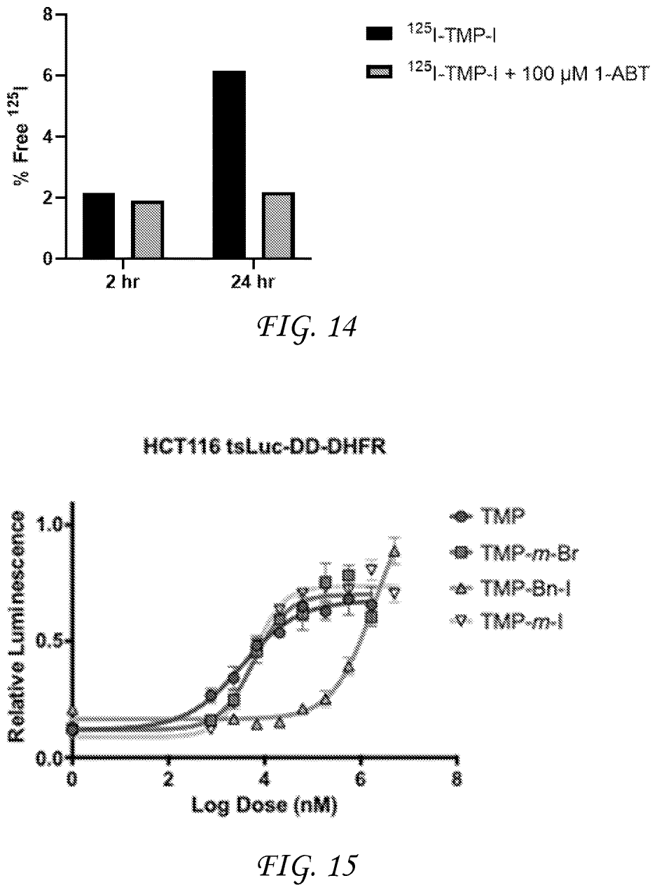

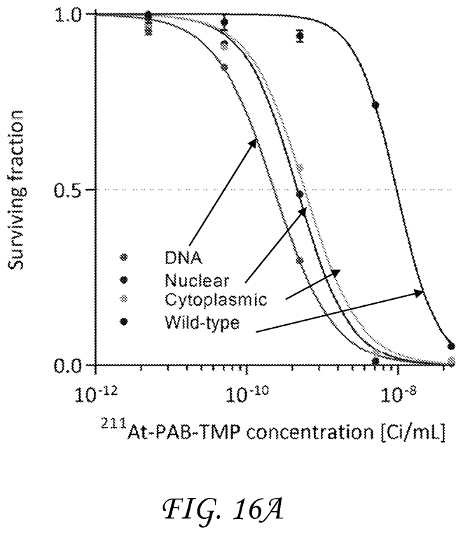

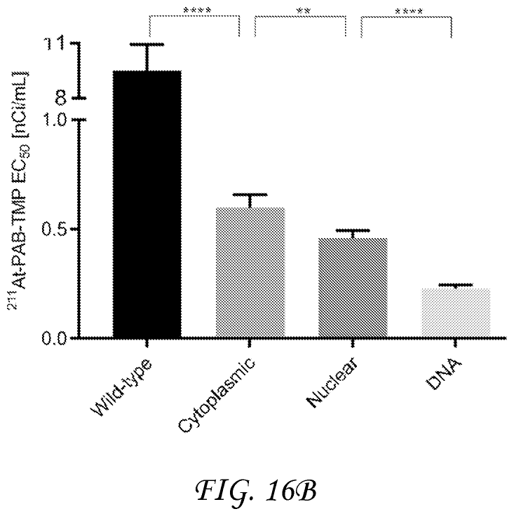

The inventors found that this compound had enhanced killing of eDHFR expressing neuroblastoma cells. In some embodiments, the inventors hypothesize that this is due to the localization of eDHFR to the nucleus of the neuroblastoma cells. The inventors show that in HCT116 cells, the meta position iodination (mI-TMP) and meta-position bromination (mBr-TMP), has higher affinity for eDHFR binding in cells than the para-benzyl-iodo-TMP (all compounds non-radiolabeled, FIG. 15).

### Compositions Containing the Compounds

Pharmaceutical compositions useful herein contain the compounds discussed herein in a pharmaceutically acceptable carrier or diluent with other optional suitable pharmaceutically inert or inactive ingredients. In other embodiments, the compounds are present in a single composition. In a further embodiment, the compounds are combined with one or more excipients and/or other therapeutic agents as described below.

### (i) Salts

The compounds may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals.

In some embodiments, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids.

In other embodiments, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is sodium hydroxide, lithium hydroxide, potassium hydroxide, or mixtures thereof.

### (ii) Carriers and Diluents

The pharmaceutical compositions include a compound described herein formulated neat or with one or more pharmaceutical carriers for administration, the proportion of which is determined by the solubility and chemical nature of the compound, chosen route of administration and standard pharmacological practice. The pharmaceutical carrier may be solid or liquid.

Although the compound may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions.

When liquid carriers are utilized, they are desirably sterile liquids. Liquid carriers are typically utilized in preparing solutions, suspensions, emulsions, syrups and elixirs. In one embodiment, the compound is dissolved a liquid carrier. In another embodiment, the compound is suspended in a liquid carrier. One of skill in the art of formulations would be able to select a suitable liquid carrier, depending on the route of administration. In one embodiment, the liquid carrier includes, without limitation, water, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, the compound may be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable excipients, including those described below.

Examples of excipients which may be combined with the compound include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients,” 5^{th} Edition, Eds.: Rowe, Sheskey, and Owen, APhA Publications (Washington, DC), Dec. 14, 2005, which is incorporated herein by reference.

### Methods of Using the Compound

As discussed herein, the radiolabeled compounds described herein are useful in delivering radiation to a patient in need thereof. In doing so, they have potential in a wide scope of applications such as treating cancer. The compounds described herein target cells or tissue that express dihydrofolate reductase (DHFR). Thus, by delivering the compounds to such cells and/or tissues (e.g., contacting the cells and/or tissues with the compounds described herein), it is possible to deliver radiation to the DHFR expressing cells or, thereby resulting in death of the DHFR expressing cells. It also is possible to deliver radiation to cells or tissues that are adjacent to DHFR expressing cells or tissues are killed, thereby also resulting in death of the adjacent cells or tissue.

“Cancer” as used herein, refers to neoplastic cells in a patient which have abnormal cell group and invade or have the potential to invade one or more body parts of the patient. In some embodiments, the cancer is a neuroendocrine cancer. In other embodiments, the cancer is of the adrenal gland, appendix, bladder, blood, brain, bone, breast, bronchus, central nervous system, cervix, chest, colon, esophagus, eye, gallbladder, head, intestines, kidney, larynx, liver, lung, lymph nodes, mouth, neck, ovaries, pancreas, pharynx, pituitary, prostate, rectum, skin, stomach, testicles, throat, thymus, thyroid, uterus, urinary tract, or vagina, or is a leukemia. In further embodiments, the cancer is a solid cancer, such as a tumor.

The treatment methods include genetically engineering cells from the subject patient to express dihydrofolate reductase. The engineered cells may then be tagged with a compound described herein. In some embodiments, the cells are transgenic cells carrying DHFR, such as E. coli DHFR. The genetically engineered and tagged cells may then be administered to the patient. Such genetic engineering may be performed using skill in the art. In certain embodiments, the genetically engineered cells are T-cells, NK-cells, macrophages, B-cells, stem cells, hematopoietic stem cells, mesenchymal stem cells, neuroprogenitor cells, induced pluripotent cells, or any combinations thereof. In other embodiments, the genetically engineered cells are CAR T-cells. In further embodiments, Ec DHFR is fused to a C-terminus of the signaling CD3zeta domain of the fibroblast activation protein (FAP) of a CAR T-cell. The inventors found that the use of CAR T-cells resulted in more selective uptake in cells as compared to using other genetically engineering cells.

DIFR may be delivered to the target cells of interest using skill in the art. In some embodiments, DI-FR may be delivered using lentivirus, naked DNA, encapsulated DNA, naked RNA, encapsulated RNA, or a viral vector. In some embodiments, DIFR is delivered to the target cells using lentivirus such as lentiviral engineering of an adoptive cell therapy such as CRISPR or similar knock-in modifications of genomes. In other embodiments, DIFR is delivered using naked DNA. In further embodiments, DIFR is delivered using encapsulated RNA. In yet other embodiments, DIFR is delivered using naked RNA. In still further embodiments, DIFR is delivered using encapsulated RNA. In other embodiments, DHFR is delivered using a viral vector such as an oncolytic virus or AAV.

Desirably, the genetically engineered cells express DHFR. The genetically engineered cells may be bacterial or mammalian. In some embodiments, the genetically engineered cells are bacterial. In other embodiments, the genetically engineered cells are mammalian. Bacterial cells may be commensal or infectious. In some embodiments, the bacterial cells are commensal. In other embodiments, the bacterial cells are infectious. In further embodiments, the bacteria cells, e.g., bacterial cells expressing DHFR, are E. coli (Ec DI-FR or eDHFR), S. aureus, P. aureginosa, Enterobacter, Haemophilus, Klebsiella, Morganella, Proteus, Providencia, Salmonella, Serratia, Streptococcus A, Streptococcus B, Streptococcus C, Streptococcus G, Mycobacterium TB, or any combination thereof.

In some embodiments, the disclosure provides methods of killing a cell or tissue comprising E. coli dihydrofolate reductase (eDHFR), comprising exposing the cell or tissue to one or more compounds described herein.

In other embodiments, the disclosure provides methods of treating a cancer in a subject in need thereof. The methods include (a) delivering eDHFR to cells or tissue surrounding the cancer as described herein and (b) administering an effective amount of a compound described herein to said subject.

In addition to the compounds described herein, another therapeutic agent may be administered to the subject. In some embodiments, the another therapeutic agent is an expressed protein or immunotherapy protein. In some embodiments, the another therapeutic agent is an expressed protein such as CAR-eDHFR. In yet other embodiments, the another therapeutic agent is an immunotherapy protein such as an antibody, mini body, diabody, or a cytokine such as IL-2 or IL-12.

The methods may also include monitoring radiation delivery of the compounds using imaging. One of skill in the art would be able to select a suitable imagining technique including, e.g., single photon emission computed tomography (SPECT).

A therapeutically or prophylactically effective amount of a compound is that amount of a compound which provides a sufficient amount of radiation. The effective amount of may be determined by the attending physician, formulation and route of delivery, condition treated, compound, route of delivery, age, weight, severity of the patient's symptoms, and response pattern of the patient. In one embodiment, effective amount does not exceed normal organ dose limits. In one embodiment, the effective amount is about 0.01 mg/kg to 10 mg/kg body weight. In another embodiment, the effective amount is less than about 5 μg/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg.

A therapeutically or prophylactically effective amount of a compound may also be that amount of a compound which provides a sufficient amount of radiation. The sufficient amount of radiation may vary depending upon the formulation and route of delivery. In one embodiment, the amount (i.e., per unit) of the compound is that which does not exceed normal organ dose limits. In one embodiment, the compound delivers about 1 μCi to about 100 mCi of radiation. In other embodiments, the compound delivers about 1 μCi to about 50 mCi, or about 1 μCi to about 10 mCi, of radiation. However, the effective amount to be used is subjectively determined by the attending physician and variables such as the size, age and response pattern of the patient.

These effective amounts may be provided on regular schedule, i.e., daily, weekly, monthly, or yearly basis or on an irregular schedule with varying administration days, weeks, months, etc. Alternatively, the effective amount to be administered may vary. In one embodiment, the effective amount for the first dose is higher than the effective amount for one or more of the subsequent doses. In another embodiment, the effective amount for the first dose is lower than the effective amount for one or more of the subsequent doses.

The compound may be administered by any desirable route, taking into consideration the specific condition for which it has been selected. The compound may, therefore, be delivered orally, by injection, i.e., transdermal, intravenous, subcutaneous, intramuscular, intravenous, intra-arterial, intraperitoneal, intrathecal, intracavitary, or epidural, among others.

### Targeted Subcellular Therapy

Targeted alpha therapy (TAT) refers to a novel class of radiopharmaceutical therapy (RPT) using an alpha particle-emitting radioisotope. Compared to the currently available clinical RPT that uses high-energy beta emitters, TAT offers several advantages that support its therapeutic potential.

It was found that the disclosed compounds target subcellular components of cells that have been engineered with DHFR. By doing so, the compounds cause irreparable DNA damage to cells, thereby producing unusually high cytotoxicity. Desirably, the targeted cells are cancer cells. Moreover, the compounds are able to selectively target cancer cells over the patient's normal cells. In some embodiments, the compounds target the cell nucleus. In other embodiments, the compounds target cellular DNA. In further embodiments, the compounds disrupt the cell membrane. Thus, in some embodiments, the compounds may be useful in treating patients who failed conventional beta-emitting RPT.

The disclosure also provides methods of engineering a radioligand binding domain to target a nucleus or DNA of a cell. By doing so, a target protein (ligand binding domain) can be engineered to different subcellular locales for the purpose of radioligand therapy. Advantageously, the method result in increasing cytotoxicity of the cell when a radioligand is delivered to the cell. In some embodiments, the radioligand is a compound described herein.

In some embodiments, the methods target DNA binding of the radioligand. For example, DNA binding may be targeted by fusing Sulfolobus solfataricus Sso7d DNA-binding domain, such as the amino acid sequence of MATVKFKYKGEEKEVDISKIKKVWRVGKMISFTYDEGGGKTGRGAVSEKDAPKE LLQMLEKQKK. In other embodiments, the methods target nucleus binding of the radioligand. For example, nucleus binding is targeted by fusing a Simian virus 40 large tumor antigen nuclear localization signal, such as the amino acid sequence PKKKRKV, or a Nucleoplasmin nuclear localization signal, such as the amino acid sequence KRPAATKKAGQAKKKK.

The methods comprise fusing a nucleic acid encoding the radioligand binding domain to a nucleic acid encoding a tag that spatially constrains the location of the radioligand binding domain to a specific subcellular locale to provide a fusion genetic construct. In some embodiments, the radioligand binding domain is eDHFR.

The methods also comprise expressing one or more proteins of the fusion genetic construct in the cell.

The methods may also include administering a radioligand to the cell. In some embodiments, the cell is in a patient. In other embodiments, the radioligand is selected from any of the compounds described herein.

### CAR T Cells

In some embodiments, the compounds may be used in conjunction with CAR-T therapy in the treatment of neoplastic disease. By doing so, the compounds modulates the toxicity of CAR-T therapy and act as a kill switch (e.g., cyto-reductive agent) for CAR-T therapy to abrogate toxicities or potentially as a combination therapy with adoptive cell treatments. Because CAR T cells show robust and selective uptake of the tracer, the compounds of the disclosure selectively target CAR T cells expressing eDHFR. By doing so, CAR T cells are ablated and near-by tumor cells are irradiated and are more effective eradicated or shrunk as compared to other therapies in the art.

In certain embodiments, the CAR T cells target fibroblast activation protein (FAP) which is a widely overexpressed cancer fibroblast marker. See, e.g., U.S. Patent Publication No. US-2020/0316231. The FAP-eDHFR CAR T cell targets the tumor stroma, rather than the tumor cells themselves. In some embodiments, once the CAR T cells are no longer needed, they can be ablated using the compounds of the disclosure which target the eDHFR present therein. In other embodiments, FAP-eDHFR CAR T cells have a synergistic effect with the compounds of the disclosure. Thus, while FAP-targeted CAR T cells can reduce extracellular matrix in tumors, they do not eradicate tumors. FAP-targeted CAR T cells, however, can deliver a the compounds of the disclosure to the TME to ablate any remaining tumor cells. In some embodiments FAP-targeted CAR T cells are transient in the body and, thus, can have continued therapeutic applications weeks after administration, e.g., at least 1 or 2 weeks after administration.

“Activation,” as used herein, refers to the state of a T cell that has been sufficiently stimulated to induce detectable cellular proliferation. Activation can also be associated with induced cytokine production, and detectable effector functions. “Activated T cells” refers to, among other things, T cells that are undergoing cell division.

“Antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies may exist in a variety of forms including, e.g., polyclonal antibodies, monoclonal antibodies, Fv, Fab and F(ab)₂, as well as single chain antibodies (scFv) and humanized antibodies.

“Antigen” or “Ag” as used herein is defined as a molecule that provokes an immune response. This immune response may involve either antibody production, or the activation of specific immunologically-competent cells, or both. The skilled artisan will understand that any macromolecule, including, e.g., proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have specificity to a selected target, e.g. a B cell surface receptor. CARs may also comprise an intracellular activation domain, a transmembrane domain and an extracellular domain comprising a tumor associated antigen binding region. In some aspects, CARs comprise an extracellular domain comprising an anti-B cell binding domain fused to CD3-zeta transmembrane and intracellular domain.

“Cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system and “exogenous” refers to any material introduced from or produced outside an organism, cell, tissue or system.

“Expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are about 50% homologous; if about 90% of the positions, are matched or homologous, the two sequences are about 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. In some embodiments, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. See, e.g., Jones, Nature, 321: 522-525, 1986; Reichmann, Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

“Immune response” as used herein is a cellular response to an antigen that occurs when lymphocytes identify antigenic molecules as foreign and induce the formation of antibodies and/or activate lymphocytes to remove the antigen.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, e.g., a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

“Modulating,” as used herein, means mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Overexpressed” tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

“Promoter” as used herein is defined as a DNA sequence recognized by the synthetic machinery of the cell, or introduced synthetic machinery, required to initiate the specific transcription of a polynucleotide sequence.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

“Specifically binds,” as used herein with respect to an antibody, refers to an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. If an antibody is specific for epitope “A,” the presence of a molecule containing epitope A (or free, unlabeled A), in a reaction containing labeled “A” and the antibody, will reduce the amount of labeled A bound to the antibody.

A “stimulatory molecule,” as used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this refers simply to cell that have been separated from the cells with which they are naturally associated in their natural state. In some embodiments, the cells are cultured in vitro. In other embodiments, the cells are not cultured in vitro.

A “ligand binding domain” as used herein means a domain that binds a ligand, e.g. a radiolabeled tracer or a small molecule suicide switch.

As used herein, “T cell receptor” or “TCR” refers to a complex of membrane proteins that participate in the activation of T cells in response to the presentation of antigen.

“Transfected” or “transduced” as used herein refers to a process by which exogenous nucleic acid is transferred or introduced into the host cell. A “transfected” or “transduced” cell is one which has been transfected or transduced with exogenous nucleic acid. The cell includes the primary subject cell and its progeny.

“Operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, “vector” includes an autonomously replicating plasmid or a virus. This term should also be construed to include non-plasmid and non-viral compounds which facilitate transfer of nucleic acid into cells, such as, e.g., polylysine compounds, liposomes, and the like. Examples of viral vectors include, but are not limited to, Sendai viral vectors, adenoviral vectors, adeno-associated virus vectors, retroviral vectors, lentiviral vectors, and the like.

The methods described herein may use cells comprising a chimeric antigen receptor (CAR) and further comprising a nucleic acid molecule comprising a ligand binding domain capable of binding to a radiolabeled tracer. In one embodiment, the cells are T cells.

In further aspects, the disclosure provides uses of cells known in the art that can be genetically modified. In some embodiments, the genetically engineering cell is a T-cell, NK-cell, macrophage, B-cell, stem cell, hematopoietic stem cell, mesenchymal stem cell, neuroprogenitor cell, induced pluripotent cell, or any combination thereof.

In some embodiments, the methods comprise administering to the subject an engineered T cell comprising a chimeric antigen receptor (CAR) and a nucleic acid molecule comprising a ligand binding domain, administering to the subject a radiolabeled tracer capable of binding to the ligand binding domain; and, detecting the amount of radiolabeled tracer bound by imaging.

### A. Radioligand Binding Domain for RLT

Some of the potential side effects of CAR T cells can be overcome by the co-expressing a radioligand binding domain in the CAR T cell. According to the disclosure, a compound of the disclosure is added to the engineered CAR T cells and these cells are then selectively removed in case toxicity arises in the subject or after targeting B cells for depletion when treating a disease or condition.

The methods include use of an isolated nucleic acid encoding a radioligand binding domain. Examples of radioligand binding domains include proteins that cause selective accumulation of radioactive molecules, and include but are not limited to, Herpes Simplex Virus thymidine kinase (HSV-tk) and [¹³¹I]FIAU and sodium iodide symporter (NIS) and [¹³¹I]NaI.

The radioligand binding domain may be operably linked to a promoter, such as an inducible promoter sequence. Examples of inducible promoters include, but are not limited to, a heat shock promoter, a tetracycline-regulated promoter, a steroid-regulated promoter, a metal-regulated promoter, an estrogen receptor-regulated promoter, and others known in the art. In one aspect, an isolated nucleic acid sequence comprising a nucleic acid sequence encoding a radioligand binding domain and a nucleic acid encoding a chimeric antigen receptor may be used. In another aspect, an isolated nucleic acid sequence encoding a radioligand binding domain and a nucleic acid encoding a chimeric antigen receptor may be used.

In some embodiments, the radioligand binding domain is under the control of an inducible promoter.

In further embodiments, the radioligand binding domain is in an expression vector. In an exemplary embodiment, a vector comprising a nucleic acid sequence comprising a radioligand binding domain may be used. The expression vector may also include other genes, such as a chimeric antigen receptor and/or CRISPR system.

The disclosure also includes cells comprising the radioligand binding domain. In an exemplary aspect, a modified cell comprising a nucleic acid comprising a radioligand binding domain and a nucleic acid encoding a chimeric antigen receptor may be used.

In other embodiments, expression of the radioligand binding domain is activated in the cell by contacting the cell with an inducing agent administered to the cell or to a subject comprising the cell. The inducing agent then activates an inducible promoter to express the radioligand binding domain. In such an embodiment, the inducing agent is administered to the subject to induce expression of the radioligand binding domain.

### B. Chimeric Antigen Receptor (CAR)

Described herein are engineered cells with a CAR and a radioligand binding domain and a ligand comprising a radiolabeled tracer. Also described are nucleic acids encoding a CAR or an engineered cell (e.g. T cell) comprising a CAR, wherein the CAR includes an antigen binding domain, a transmembrane domain and an intracellular domain.

One or more domains or a fragment of a domain of the CAR may be human. In one embodiment, the disclosure provides a fully human CAR. The nucleic acid sequences coding for the desired domains can be obtained using recombinant methods known in the art. Alternatively, the gene of interest can be produced synthetically, rather than as a cloned molecule.

Examples of CARs are described in U.S. Pat. Nos. 8,911,993, 8,906,682, 8,975,071, 8,916,381, 9,102,760, 9,101,584, and 9,102,761, all of which are incorporated herein by reference in their entireties.

### B. Antigen Binding Domain

In some embodiments, the CAR comprises an antigen binding domain that binds to a B cell. Cell surface markers selectively found on B cells may act as an antigen that binds to the antigen binding domain of the CAR.

The anti-B cell antigen binding domain can include any domain that binds to the B cell and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof, such as a single chain variable fragment (scFv).

The antigen binding domain may bind one or more B cell antigens, such as, but not limited to, any surface marker selectively found on a B cell, such as a pro-B cell, pre-B cell, immature B cell, mature B cell, memory B cell, and plasma cell. In one embodiment, the antigen binding domain binds at least one B cell antigen, such as CD19, BCMA, and any combination thereof. In another embodiment, the antigen binding domain binds at least one B cell antigen, such as CD20, CD21, CD27, CD38, CD138, and any combination thereof.

In some instances, it is beneficial for the antigen binding domain to be derived from the same species in which the CAR will ultimately be used in. For example, for use in humans, it may be beneficial for the antigen binding domain of the CAR to comprise a human antibody, humanized antibody as described elsewhere herein, or a fragment thereof.

It is also beneficial that the antigen binding domain is operably linked to another domain of the CAR, such as the transmembrane domain or the intracellular domain, both described elsewhere herein, for expression in the cell. In one embodiment, a nucleic acid encoding the antigen binding domain is operably linked to a nucleic acid encoding a transmembrane domain and a nucleic acid encoding an intracellular domain.

In addition to B cell antigen binding domains, the antigen binding domain can be any antigen binding domain suitable for introduction into a CAR construct, where binding of the antigen binding domain to its cognate binding partner has a beneficial effect on a subject.

### C. Transmembrane Domain

With respect to the transmembrane domain, the CAR can be designed to comprise a transmembrane domain that connects the antigen binding domain of the CAR to the intracellular domain. In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

### D. Intracellular Domain

The intracellular domain or otherwise the cytoplasmic domain of the CAR is responsible for activation of the cell in which the CAR is expressed. “Intracellular domain” is thus meant to include any portion of the intracellular domain sufficient to transduce the activation signal. In one embodiment, the intracellular domain includes a domain responsible for an effector function. “Effector function” refers to a specialized function of a cell. Effector function of a T cell, e.g., may be cytolytic activity or helper activity including the secretion of cytokines.

In some embodiments, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of intracellular domains include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability. In one embodiment, the intracellular domain of the CAR comprises dual signaling domains. The dual signaling domains may include a fragment or domain from any of the molecules described herein.

Examples of intracellular domain includes a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rb), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In other embodiments, the intracellular domain of the CAR includes any portion of a co-stimulatory molecule, such as at least one signaling domain from CD3, CD27, CD28, CD83, CD86, CD127, 4-1BB, 4-1BBL, PD1, PD1L, T cell receptor (TCR), any derivative or variant thereof, any synthetic sequence thereof that has the same functional capability, and any combination thereof.

Between the antigen binding domain and the transmembrane domain of the CAR, or between the intracellular domain and the transmembrane domain of the CAR, a spacer domain may be incorporated. As used herein, “spacer domain” generally means any oligo- or polypeptide that functions to link the transmembrane domain to, either the antigen binding domain or, the intracellular domain in the polypeptide chain. In one embodiment, the spacer domain may comprise up to about 300 amino acids, preferably about 10 to about 100 amino acids and most preferably about 25 to about 50 amino acids. In another embodiment, a short oligo- or polypeptide linker, preferably between about 2 and about 10 amino acids in length may form the linkage between the transmembrane domain and the intracellular domain of the CAR. An example of a linker includes a glycine-serine doublet.

In some embodiments, the CAR further comprises a signal peptide.

In some embodiments, the CAR further comprises E. coli dihydrofolate reductase (eDHFR) or similar molecule. In such an embodiment, the presence of CAR molecules on the surface of the modified T cell is prevented by spontaneous aggregation of the CAR molecules in the cytoplasm or other internal location in the cell. Between the antigen binding domain and the transmembrane domain of the CAR, or between the intracellular domain and the transmembrane domain of the CAR, a spacer domain may be incorporated.

In some embodiments, the CAR further comprises a signal peptide.

### E. CRISPR/Cas

The T cells described herein made be modified by deleting the endogenous T cell receptor (TCR) and/or major histocompatibility complex (MHC) molecules with genome editing technology to reduce or prevent the transmission of stimulatory signals through the endogenous TCR/MHC complex.

The CRISPR/CAS system can be engineered to cleave virtually any DNA sequence by redesigning the gRNA in cell lines (such as 293T cells), primary cells, and CAR T cells. The CRISPR/CAS system can simultaneously target multiple genomic loci by co-expressing a single CAS9 protein with two or more gRNAs, making this system uniquely suited for multiple gene editing or synergistic activation of target genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, the CRISPR system comprises an expression vector, such as, but not limited to, an pAd5F35-CRISPR vector. In one embodiment, the modified T cell described herein is further modified by introducing a Cas expression vector and a guide nucleic acid sequence specific for a gene into the modified T cell. In another embodiment, the Cas expression vector induces expression of Cas9 endonuclease. Other endonucleases may also be used including, but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence may be specific for a T cell receptor (TCR) chain (such as an alpha, beta, gamma and/or delta chain), a major histocompatibility complex protein (such as a HLA class I molecule and/or HLA class II molecule), and any combination thereof.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In some embodiments, a T cell is modified to express a CAR and the CAR T cell is further modified to delete endogenous TCR or MHC molecules, such as before administration to a subject. In one embodiment, the CAR modified T cell described herein is further modified by deleting a gene that is a T cell receptor (TCR) chain, a major histocompatibility complex protein, or any combination thereof. In another embodiment, the T cell is modified before administration to the subject in need thereof.

In some embodiments, a T cell is modified to express a CAR, administered to a subject, and then further modified in vivo to delete endogenous TCR or MHC molecules, such as through inducing targeted gene deletion. In one embodiment, the modified T cell described herein is modified by inducing a CRISPR/Cas system to minimize native reactivity of the modified T cell or host reactivity to the modified T cell. In some embodiments, inducing the Cas expression vector comprises exposing the modified T cell to an agent that activates an inducible promoter in the Cas expression vector. In such an embodiment, the Cas expression vector includes an inducible promoter, such as one that is inducible by exposure to an antibiotic (e.g., by tetracycline or a derivative of tetracycline, e.g. doxycycline). However, it should be appreciated that other inducible promoters can be used. The inducing agent can be a selective condition (e.g., exposure to an agent, e.g. an antibiotic) that results in induction of the inducible promoter. This results in expression of the Cas expression vector.

In some embodiments, a T cell is modified to delete endogenous TCR or MHC molecules prior to modification to express the CAR. In some embodiments, the modified T cell is further modified by deleting TCR or MHC molecules prior to inducing expression of the radioligand binding domain.

### F. Introduction of Nucleic Acids

Methods of introducing nucleic acids into a cell include physical, biological and chemical methods.

In one embodiment, the nucleic acids introduced into the T cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, e.g., genomic DNA, plasmid DNA, phage DNA, cDNA, synthetic DNA sequence or any other appropriate source of DNA. The desired template for in vitro transcription is a chimeric membrane protein. By way of example, the template encodes an antibody, a fragment of an antibody or a portion of an antibody. By way of another example, the template comprises an extracellular domain comprising a single chain variable domain of an antibody, such as anti-CD3, and an intracellular domain of a co-stimulatory molecule. In one embodiment, the template for the RNA chimeric membrane protein encodes a chimeric membrane protein comprising an extracellular domain comprising an antigen binding domain derived from an antibody to a co-stimulatory molecule, and an intracellular domain derived from a portion of an intracellular domain of CD28 and 4-1BB.

PCR can be used to generate a template for in vitro transcription of mRNA which is then introduced into cells.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, Trends in Biochem. Sci., 29:436-444 (2001); Stepinski, RNA, 7:1468-95 (2001); Elango, Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

Some IVT vectors may be utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

### G. Sources of T Cells

Prior to expansion, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, e.g., Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, e.g., by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an

### Kits Containing the Compound

Also provided herein are kits or packages of pharmaceutical formulations containing a one or compound or composition described herein. The kits may be organized to indicate a single formulation or combination of formulations to be taken at each desired time. The composition may also be sub-divided to contain appropriate quantities of the compound. For example, the unit dosage can be packaged compositions, e.g., packeted powders, vials, ampoules, prefilled syringes or sachets containing liquids.

Suitably, the kit contains packaging or a container with the compound formulated for the desired delivery route. Suitably, the kit contains instructions on dosing and an insert regarding the compound. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The compound or composition described herein can be a single dose or for continuous or periodic discontinuous administration. For continuous administration, a package or kit can include the compound in each dosage unit, e.g., solution, lotion, tablet, pill, or other unit described above or utilized in drug delivery. When the compound is to be delivered with periodic discontinuation, a package or kit can include placebos during periods when the compound is not delivered. When varying concentrations of a composition, of the components of the composition, or of relative ratios of the compound or other agents within a composition over time is desired, a package or kit may contain a sequence of dosage units, so varying.

A number of packages or kits are known in the art for the use in dispensing pharmaceutical agents for oral use. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, such as the lungs, injected into a subject, or even applied to and mixed with the other components of the kit.

The compound or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In one embodiment, a pharmaceutical kit is provided and contains one or more compounds of formula I, II, and III. The compound may be in the presence or absence of one or more of the carriers or excipients described above. The kit may optionally contain a chemotherapeutic and/or instructions for administering the chemotherapeutic and the compound to a subject having cancer.

In a further embodiment, a pharmaceutical kit is provided and contains a chemotherapeutic in a first dosage unit, one or more of a compound described herein in a second dosage unit, and one or more of the carriers or excipients described above in a third dosage unit. The kit may optionally contain instructions for administering the chemotherapeutic and/or compound to a subject having cancer.

### Aspects

Aspect 1. A compound of formula I:

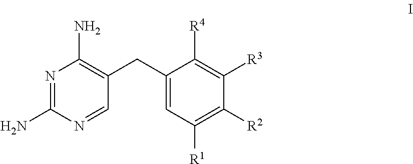

wherein:
   R¹, R³, and R⁴ are, independently, H or radioactive halo; and R² is H, halo, C₁₋₆alkoxy, C₁₋₆alkyl, or aryl, provided that at least one of R¹, R³, and R⁴ is radioactive halo; or a pharmaceutically acceptable salt thereof.

Aspect 2. The compound of Aspect 1, wherein the radioactive halo is ¹⁸F, ³²Cl, ³³C, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I, or ²¹¹At.

Aspect 3. The compound of Aspect 1, wherein R¹, R³, or R⁴ is ¹²⁵I.

Aspect 4. The compound of Aspect 1, wherein R¹, R³, or R⁴ is ¹³¹I.

Aspect 5. The compound of Aspect 1, wherein R¹, R³, or R⁴ is ²¹¹At.

Aspect 6. The compound of Aspect 1, wherein R¹, R³, or R⁴ is ⁷⁵Br.

Aspect 7. The compound of Aspect 1, wherein R¹, R³, or R⁴ is ⁷⁶Br.

Aspect 8. The compound of Aspect 1, wherein R¹, R³, or R⁴ is ⁷⁷Br.

Aspect 9. The compound of any one of Aspects 1-8, wherein R² is H.

Aspect 10. The compound of any one of Aspects 1-8, wherein R² is C₁₋₆alkoxy, such as methoxy.

Aspect 11. The compound of any one of Aspects 1-8, wherein R² is C₁₋₆alkyl, such as methyl.

Aspect 12. The compound of any one of Aspects 1-8, wherein R² is aryl such as phenyl.

Aspect 13. The compound of any one of Aspects 1-8, wherein R² is halo such as F, Cl, Br, or I.

Aspect 14. The compound of Aspect 1, that is of formula I-A or I-B:

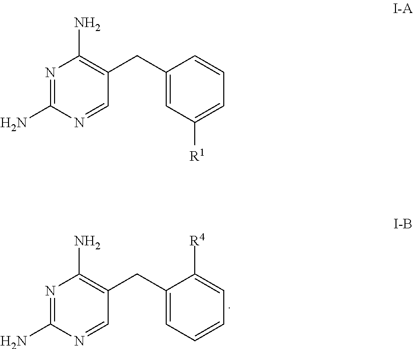

Aspect 15. The compound of Aspect 1 that is

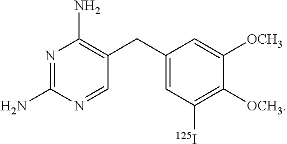 [SMILES: COc1cc(Cc2cnc(N)nc2N)cc([125I])c1OC]

Aspect 16. The compound of Aspect 1 that is

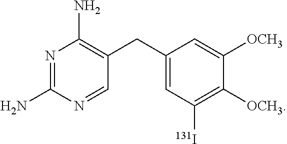 [SMILES: COc1cc(Cc2cnc(N)nc2N)cc([131I])c1OC]

Aspect 17. The compound of Aspect 1 that is

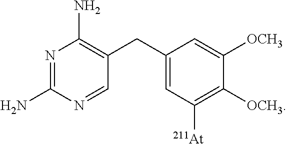

Aspect 18. The compound of Aspect 1 that is

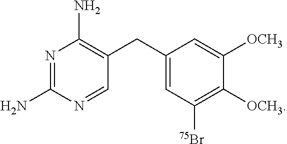 [SMILES: COc1cc(Cc2cnc(N)nc2N)cc([75Br])c1OC]

Aspect 19. The compound of Aspect 1 that is

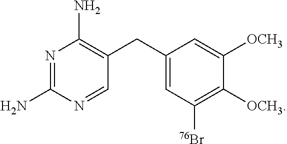 [SMILES: COc1cc(Cc2cnc(N)nc2N)cc([76Br])c1OC]

Aspect 20. The compound of Aspect 1 that is

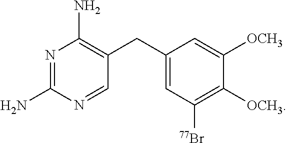 [SMILES: COc1cc(Cc2cnc(N)nc2N)cc([77Br])c1OC]

or a pharmaceutically acceptable salt thereof.

Aspect 21. A compound of formula II:

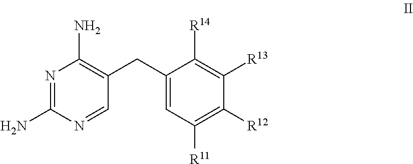

wherein: R¹² is a radioactive halo; and R¹¹, R¹³, and R¹⁴ are, independently, H, C₁₋₆alkoxy, C₁₋₆alkyl, or aryl, provided that at least one of R¹¹, R¹³ and R¹⁴ is C₁₋₆alkyl or aryl; or a pharmaceutically acceptable salt thereof.

Aspect 22. The compound of Aspect 20, wherein the radioactive halo is ¹⁸F, ³²Cl, ³³Cl, ³⁴Cl, ⁷⁴Br, ⁷⁵Br, ⁷⁶Br, ⁷⁷Br, ⁷⁸Br, ¹²³I, ¹²⁵I, ¹²⁴I, ¹³¹I, or ²¹¹At.

Aspect 23. The compound of Aspect 21, wherein R¹² is ¹²⁵I.

Aspect 24. The compound of Aspect 21, wherein R¹² is ¹³¹I.

Aspect 25. The compound of Aspect 21, wherein R¹² is ²¹¹At.

Aspect 26. The compound of Aspect 21, wherein R¹² is ⁷⁵Br.

Aspect 27. The compound of Aspect 21, wherein R¹² is ⁷⁶Br.

Aspect 28. The compound of Aspect 21, wherein R¹² is ⁷⁷Br.

Aspect 29. The compound of any one of Aspects 20-27, wherein R¹¹ is H.

Aspect 30. The compound of any one of Aspects 20-27, wherein R¹¹ is C₁₋₆alkoxy, such as methoxy.

Aspect 31. The compound of any one of Aspects 20-27, wherein R¹¹ is C₁₋₆alkyl, such as methyl.

Aspect 32. The compound of any one of Aspects 20-27, wherein R¹¹ is aryl such as phenyl.

Aspect 33. The compound of any one of Aspects 20-31, wherein R¹³ is H.

Aspect 34. The compound of any one of Aspects 20-31, wherein R¹³ is C₁₋₆alkoxy, such as methoxy.

Aspect 35. The compound of any one of Aspects 20-31, wherein R¹³ is C₁₋₆alkyl, such as methyl.

Aspect 36. The compound of any one of Aspects 20-31, wherein R¹³ is aryl such as phenyl.

Aspect 37. The compound of any one of Aspects 20-35, wherein R¹⁴ is H.

Aspect 38. The compound of any one of Aspects 20-35, wherein R¹⁴ is C₁₋₆alkoxy, such as methoxy.

Aspect 39. The compound of any one of Aspects 20-35, wherein R¹⁴ is C₁₋₆alkyl, such as methyl.

Aspect 40. The compound of any one of Aspects 20-35, wherein R¹⁴ is aryl such as phenyl.

Aspect 40. A compound of formula III:

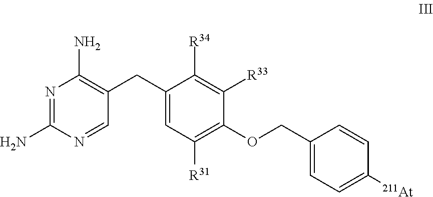

wherein R³¹, R³³, and R³⁴ are, independently, H, C₁₋₆alkoxy, C₁₋₆alkyl, or aryl; or a pharmaceutically acceptable salt thereof.

Aspect 41. The compound of Aspect 40, wherein R³¹ is H.

Aspect 42. The compound of Aspect 40, wherein R³¹ is C₁₋₆alkoxy, such as methoxy, ethoxy, propoxy, butoxy, pentoxy or hexoxy, or such as methoxy.

Aspect 43. The compound of Aspect 40, wherein R³¹ is C₁₋₆alkyl, such as methyl, ethyl, propyl, butyl, pentyl, or hexyl, or such as methyl.

Aspect 44. The compound of Aspect 40, wherein R³¹ is aryl such as phenyl.

Aspect 45. The compound of any one of Aspects 40-44, wherein R³³ is H.

Aspect 46. The compound of any one of Aspects 40-44, wherein R³³ is C₁₋₆alkoxy, such as methoxy, ethoxy, propoxy, butoxy, pentoxy or hexoxy, or such as methoxy.

Aspect 47. The compound of any one of Aspects 40-44, wherein R³³ is C₁₋₆alkyl, such as methyl, ethyl, propyl, butyl, pentyl, or hexyl, or such as methyl.

Aspect 48. The compound of any one of Aspects 40-44, wherein R³³ is aryl such as phenyl.

Aspect 49. The compound of any one of Aspects 40-48, wherein R³⁴ is H.

Aspect 50. The compound of any one of Aspects 40-48, wherein R³⁴ is C₁₋₆alkoxy, such as methoxy, ethoxy, propoxy, butoxy, pentoxy or hexoxy, or such as methoxy.

Aspect 51. The compound of any one of Aspects 40-48, wherein R³⁴ is C₁₋₆alkyl, such as methyl, ethyl, propyl, butyl, pentyl, or hexyl, or such as methyl.

Aspect 52. The compound of any one of Aspects 58-78, wherein R³⁴ is aryl such as phenyl.

Aspect 53. The compound of Aspect 40, that is

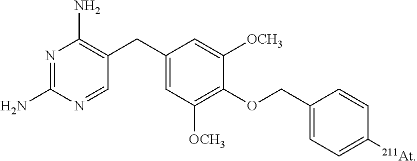

Aspect 54. A composition comprising one or more compounds of any one of Aspects 1-53 and a pharmaceutically acceptable carrier or diluent.

Aspect 55. A method of delivering radiation to dihydrofolate reductase (DHFR) expressing cells or tissues in a subject, comprising administering a compound of any one of Aspects 1-53 to the DHFR expressing cells or tissues.

Aspect 56. A method of killing a cell or tissue comprising E. coli dihydrofolate reductase (eDHFR), comprising exposing the cell or tissue to the compound of any one of Aspects 1-53.

Aspect 57. A method of treating a cancer in a subject in need thereof, comprising:
(a) delivering eDHFR to cells or tissue surrounding the cancer; and
(b) administering an effective amount of a compound of any one of Aspects 1-53 to said subject.

Aspect 58. The method of any one of Aspects 55-57, wherein the cells are bacterial or mammalian.

Aspect 59. The method of Aspect 58, wherein said bacteria are commensal or infectious.

Aspect 60. The method of Aspect 58 or 59, wherein said bacteria expresses DHFR.

Aspect 61. The method of Aspect 60, wherein said bacterial DHFR is E. coli, S. aureus, P. aureginosa, Enterobacter, Haemophilus, Klebsiella, Morganella, Proteus, Providencia, Salmonella, Serratia, Streptococcus A, Streptococcus B, Streptococcus C, Streptococcus G, Mycobacterium TB, or any combination thereof.

Aspect 62. The method of any one of Aspects 55-61, further comprising administering another therapeutic agent such as an expressed protein, or such as CAR-eDHFR, or such as an immunotherapy protein, or such as an antibody, mini body, diabody, or cytokine such as IL-2, IL12.

Aspect 63. The method of any one of Aspects 55-62, wherein other cells that are adjacent to DHFR expressing cells or tissues are killed.

Aspect 64. The method of any one of Aspects 55-63, further comprising imaging radiation delivery from the compound.

Aspect 65. The method of Aspect 64, wherein imaging is performed with single photon emission computed tomography.

Aspect 66. The method of any one of Aspects 55-65, wherein the cell or tissue is present in subject.

Aspect 67. The method of any one of Aspects 55-66, wherein the cell is contacted with the compound.

Aspect 68. The method of Aspect 57, wherein eDHFR is delivered using lentivirus such as lentiviral engineering of an adoptive cell therapy, naked DNA, encapsulated DNA, naked RNA, encapsulated RNA, or a viral vector such as an oncolytic virus or AAV.

Aspect 69. The method of any one of Aspects 55-68, wherein said cells are T-cells, NK-cells, macrophages, B-cells, stem cells, hematopoietic stem cells, mesenchymal stem cells, neuroprogenitor cells, or induced pluripotent cells.

Aspect 70. The method of Aspect 69, wherein the T-cells are CAR T-cells.

Aspect 71. The method of Aspect 70, wherein the eDHFR is fused to a C-terminus of the signaling CD3zeta domain of the fibroblast activation protein (FAP) of a CAR T-cell.

Aspect 72. The method of any one of Aspects 55-71, wherein said compound is administered orally, intravenously, intra-arterially, intraperitoneally, intrathecally, or intracavitarily.

The following examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C, pressure is at or near atmospheric.

Abbreviations

| | |
|---|---|
| d | doublet |
| DMSO | Dimethyl sulfoxide |
| EtOH | ethanol |
| FeCl₃ | ferric chloride |
| KOt-Bu | potassium tert-butoxide |
| LCMS | liquid chromatography/mass spectrometry |
| m | Multiplet |
| MeOH | methanol |
| NaI | sodium iodide |
| ppm | parts per million |
| Pd(PPh₃)₄ | Tetrakis(triphenylphosphine)palladium(0) |
| FeCl₃ | ferric chloride |
| s | Singlet |
| t | Triplet |

### EXAMPLES

Unless otherwise noted, chemicals were purchased from commercial suppliers at the highest purity grade available and were used without further purification. Thin layer chromatography was performed on 0.25 mm silica gel plates (60F254) using UV light as the visualizing agent. Silica gel (100-200 mesh) was used for column chromatography. Nuclear magnetic resonance spectra were recorded on a 400 MHz spectrometer equipped with a 5 mm BBFO IProbe (¹H=400 MHz and ¹³C=100 MHz), and chemical shifts are reported in δ units, ppm. All spectra were referenced internally to the residual proton resonance in CDCl₃ (δ 7.26 ppm) or with tetramethylsilane (TMS, δ 0.00 ppm) as the internal standard. Chemical shifts (6) were reported as part per million (ppm) on the δ scale downfield from TMS. ¹³C NMR spectra were referenced to CDCl₃ (δ 77.0 ppm, the middle peak). Coupling constants are expressed in Hz. High-resolution mass spectra were recorded with a micro TOF-Q analyzer spectrometer by using the electrospray mode. Target compounds and/or intermediates were characterized by LCMS using a Waters Acquity separation module.

Iodine-125 was purchased from PerkinElmer, product number NEZ033010MC, and received in a 0.1M NaOH (pH 10-12) (reductant free) solution ~17Ci(629 GBq)/mg. Iodine-131 was purchased from International Isotope Inc. USA. Radio-HPLC analysis was performed with an Agilent 1260 Infinity II HPLC system with a Flow-Count radio-HPLC detector system.

All statistical analyses were performed on Prism 9 (GraphPad). An Unpaired, two-tailed Student t test or Mann-Whitney test was applied to determine the statistical significance between two groups. For the comparison of more than two groups, a one-way ANOVA was used with appropriate post hoc testing. For all analyses, a p-value of <0.05 was considered statistically significant. Data points are presented as mean±SD.

### Example 1: Preparation of Compounds

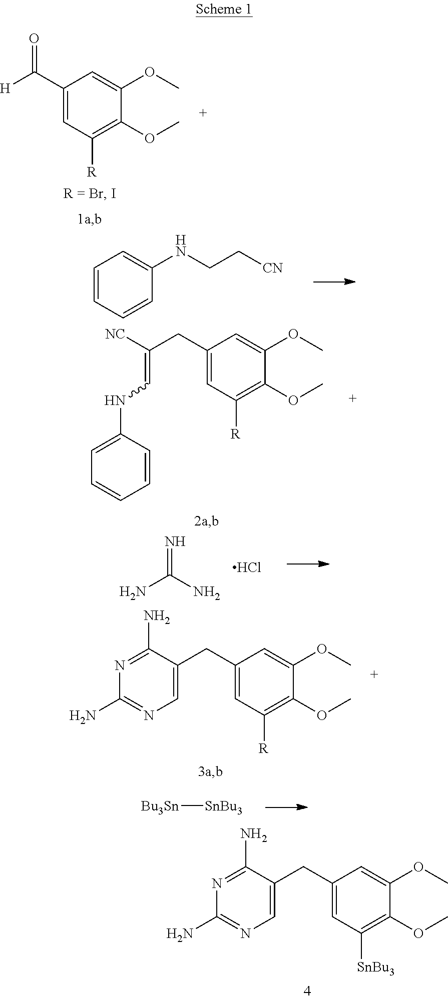

### A. Preparation of Compounds 3a and 3b (not Radiolabeled)

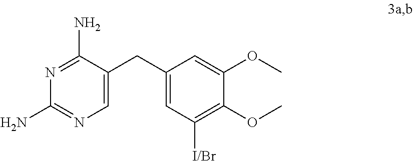

Step 1:To a solution of meta halogenated dimethoxy benzaldehyde (1a,b) (~2 g, 7.54 mmol) and 3-anilinoproprionitrile (1.3 g, 8.29 mmol, 1.1 eq) in dry DMSO (25 mL), potassium t-butoxide (972 mg, 8.68 mmol, 1.5 eq) was added and color changed to deep orange. This reaction was allowed to stir at room temperature for 1 hour. Completion of the reaction was verified by TLC and ethyl acetate (25 mL) and water (25 mL) was added. The water layer was washed with 2*10 mL of ethyl acetate. Organic layers were combined, rinsed with brine, dried over sodium sulfate and the intermediate was concentrated under rotovap. Brown oil was formed.

Step 2: In separate round bottom flask under N₂ atmosphere, anhydrous ethanol (20 mL) was added to guanidine·HCl (2.17 g, 22.67 mmol) and allowed to stir at room temperature. After 5 minutes, potassium t-butoxide (2.54 g, 22.67 mmol) and anhydrous ethanol (10 mL) were added to the solution. The reaction was allowed to stir at room temperature for 30 minutes. The solution turned into white precipitate. The solid formed was removed via vacuum filtration and filtrate was collected for the next step. The filtrate was rinsed with 5 mL cold ethanol.

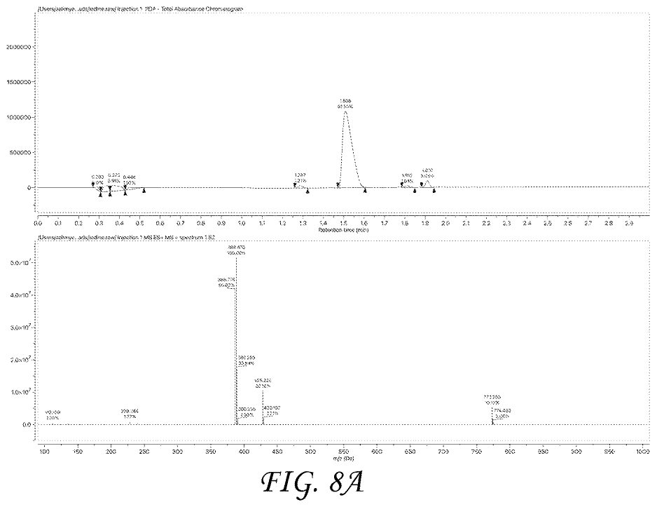

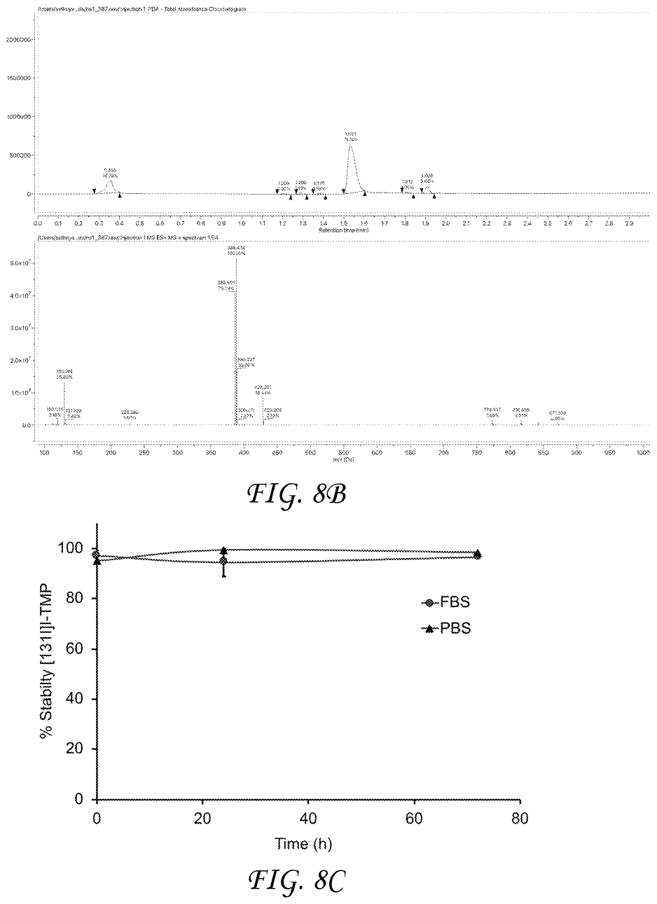

Step 3: The brown oil formed during step 1 was dissolved in minimal amounts of anhydrous ethanol, flushed with nitrogen for 10 minutes, and the clear filtrate collected from step 2 was added. The mixture was refluxed for 16 hours. The round bottomed flask was then transferred to an ice bath after cooling to room temperature for 15 minutes. A yellow precipitate began to form at the bottom of the round bottomed flask. The solid was collected via vacuum filtration and was rinsed with cold ethanol. The filtrate was extracted with ethyl acetate and water, the organic layer dried over Na₂SO₄ was concentrated, and purified by column chromatography to generate 3a (1.85 g, 74% yield) 3b (2 g, 69%). ¹H NMR (400 MHz, CDCl₃) 3a: ¹H NMR (400 MHz, CDCl₃): δ 7.56 (s, 1H), 7.14 (d, J=2.0 Hz, 1H), 6.98 (d, J=1.6 Hz, 1H), 6.11 (s, 2H), 5.71 (s, 2H), 3.77 (s, 3H), 5.71 (s, 2H), 3.77 (s, 3H), 3.65 (s, 3H), 3.53 (s, 3H). LCMS 3b m/z calcd. for C₁₃H₁₆IN₄O₂ [M+H]+: 387.031. Found: 387.470. LCMS analysis confirmed the stability of TMP-I (3a) in FBS, (FIGS. 8A-8C).

### B. Preparation of 5-(3,4-dimethoxy-5-(tributylstannyl)benzyl)pyrimidine-2,4-diamine (Compound 4)

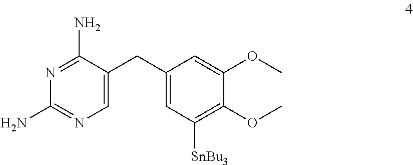

Route 1: The round bottom flask charged with TMP-meta halo derivative (0.60 mmol) and palladium catalyst (0.09 mmol, 104 mg, 0.15 equiv.) was flushed with N₂. After 10 minutes 40 mL of dry toluene was added at room temperature. 3.4 mL of Bis(tributyltin) was added to the mixture and the reaction was refluxed for 12 h. Monitor the reaction completion by LCMS. Column chromatography was performed for purification to yield 4 (52%); ¹H NMR (400 MHz, CDCl₃): δ 7.75 (s, 1H), 6.75-6.74 (m, 1H), 6.62-6.61 (m, 1H), 4.84 (s, 2H), 4.64 (s, 2H), 3.79-3.78 (m, 6H), 3.65 (s, 2H), 1.54-1.46 (m, 6H), 1.35-1.26 (m, 6H), 1.08-0.98 (m, 6H), 0.89-0.85 (m, 9H). ¹³C NMR (100 MHz, CDCl₃): δ 162.75, 161.79, 155.90, 152.17, 151.91, 136.03, 133.93, 127.56, 112.70, 107.03, 60.59, 55.43, 34.14, 29.10, 27.32, 13.66, 9.98.

Route 2: A round bottom flask charged with meta-halide TMP derivative (0.295 mmol), bis-tributyltin (1.5 mL) and palladium(0)tetrakistriphenylphosphine (51 mg, 0.15 eq.) was flushed with argon. After 10 minutes of flushing, anhydrous toluene (15 mL) was added at room temperature to the reaction mixture. The reaction mixture was then refluxed for 12 hour, the reaction was monitored by LCMS. Column chromatography was performed for purification to afford 4 (42-50% isolated yield). ¹H NMR (400 MHz, CDCl₃): δ 7.75 (s, 1H), 6.75-6.74 (m, 1H), 6.62-6.61 (m, 1H), 4.84 (s, 2H), 4.64 (s, 2H), 3.79-3.78 (m, 6H), 3.65 (s, 2H), 1.54-1.46 (m, 6H), 1.35-1.26 (m, 6H), 1.08-0.98 (m, 6H), 0.89-0.85 (m, 9H). ¹³C NMR (100 MHz, CDCl₃): δ 162.75, 161.79, 155.90, 152.17, 151.91, 136.03, 133.93, 127.56, 112.70, 107.03, 60.59, 55.43, 34.14, 29.10, 27.32, 13.66, 9.98. LCMS m/z calcd. for C₂₅H₄₃N₄O₂Sn [M+H]+ 551.240 Found: 551.200.

### C. Preparation of 5-(3-iodo-4,5-dimethoxybenzyl)pyrimidine-2,4-diamine ([^{125/131}I]m-TMP—Compound 5′)

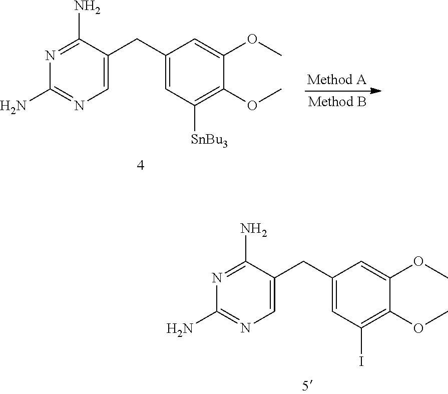

Method A: In a microcentrifuge tube, acetic acid (90 μL) in methanol (5% v/v), NaI (1.5 mM), and 27.6 μL of chloramine-T in methanol (0.1635 mM) were added and, after thoroughly mixing, 12 μL of 4 in methanol (0.62 mM) was added. The reaction was allowed to progress for 5 minutes and reaction monitored by LCMS.

Method B1:To the solution of TMP-SnBu₃ (1 mg, 1.8 μmol) in methanol (250 μl), FeCl₃ (4.3 mg, 26.5 μmol) in methanol (250 μl) was added. After vortexing, NaI (3.1 mg, 20.6 μmol) as added to the reaction mixture. The reaction progressed at room temperature for 5-7 minutes and diluted with acetonitrile and injected into HPLC using Agilent column Eclipse XDB C-18 (C18, 9.4×250 mm, 5 μm). Conditions: Mobile phase A; 0.1% TFA/H₂O (solvent A) and 0.1% TFA/I (solvent B). Gradient: 95% A to 5% B 0-8 min; 60% A to 40% B 8-18 min; 40% A to 60% B 18-30 min; 100% B 30-40 min. The labeled compounds were identified by co-elution with the non-radioactive reference compounds. See, FIGS. 7A and 7B.

Method B2:To the solution of m-SnBu₃TMP (1 mg, 1.8 μmol) in methanol (250 μl), FeCl₃ (2 mg, 12.3 μmol) in methanol (250 μl) was added. After vortexing, NaI (3.1 mg, 20.6 μmol) as added to the reaction mixture. After 5 minutes, the reaction progress was monitored by LCMS. See, FIG. 33.

Method B3:To the solution of m-SnBu₃TMP (0.5-2 mg, 0.91-3.6 μmol) in methanol (250 μl), FeCl₃ (1.0-4.3 mg, 6.62-26.5 μmol) in methanol (250 μl) was added. After vortexing, [^{125/131}I]NaI (37-185 MBq) was added to the reaction mixture. The reaction progressed at room temperature for 5-7 minutes and was then diluted with acetonitrile and injected onto a 1260 Infinity II HPLC system equipped with an Eclipse XDB-C18 4.6×250, 5μ column for purification with the mobile phase consisting of 0.1% TFA/H₂O (solvent A), 0.1% TFA/acetonitrile (solvent B) and a gradient consisting of 5% B at 0 min to 40% B in 8 min, then to 60% B in 18 min and finally to initial composition at 30 min with a flow rate of 1 mL/min. Eluents corresponding to the radiotracer were collected in 1-2 mL HPLC fractions and concentrated to 0.5 mL under nitrogen flow. It was then diluted to 5 mL with MilliQ water and loaded into a preactivated C18 Sep-Pak cartridge (Waters #WATO20515). The C-18 Sep-Pak cartridge was activated prior to use by passing 5 mL of EtOH followed by 15 mL of MilliQ water. The compound was then loaded into it and washed with 10 mL of water. Finally, radiotracer was eluted with EtOH with an isolated yield of approximately 30%. QC was performed to analyze the purity of the compound and was found to be ≥95% in all cases.

### D. HPLC Characterization

The Radio-HPLC analysis was conducted utilizing the Agilent 1260 Infinity II HPLC system, attached to Eckert and Ziegler PMT detector for precise radio peak detection. Reductant free [¹²⁵I]NaI (Perkin Elmer—product number NEZ033A005MC); specific activity of 629 GBq/mg in 10-5 M NaOH (pH 8-11) aqueous solution. [¹³¹I]NaI purchased from International Isotopes Inc., 1490-5619 mCi/mL (55.13-207.9 GBq/mL) in buffer.

Purification was performed by reverse-phase HPLC using Agilent column Eclipse XDB C-18 (C18, 9.4×250 mm, 5 μm). Conditions: Mobile phase A; 0.1% TFA/H₂O (solvent A) and 0.1% TFIACN (solvent B). Gradient: 95% A to 5% B 0-8 min; 60% A to 40% B 8-18 min; 40% A to 60% B 18-30 min; 100% B 30-40 min.

### Example 2: In Vitro Experiments

### Primary Human CAR T Cell Generation and Sorting

Primary human T cells were sourced from healthy volunteers through the Human Immunology Core at the University of Pennsylvania. All specimens were obtained following institutional review board-approved protocols and informed consent from the volunteers. Bulk T cells were activated and expanded following established methods and subsequently, either pTRPE-FAP CAR-eDHFR DF or pTRPE-FAP CAR-eDHFR DF-T2A-BFP (7) lentivirus was introduced to the activated T cells 16 hours post-activation at a multiplicity of infection (MOI) of 5. The T cells were then expanded for a period of 10 days. Transduced CAR T cells were incubated with Alexa Fluor 647 AffiniPure F(ab′2) fragment goat anti-mouse immunoglobulin G (Jackson ImmunoResearch Laboratories) for half an hour at room temperature. Following incubation, stained cells were washed 3*100 μL with FACS buffer (2% bovine serum albumin in PBS, Invitrogen). The washed cells were analyzed using a flow cytometer (LSR II, BD Biosciences) to detect CAR and/or BFP expression. FlowJo software was used to analyze the flow data.

### A. Mammalian Cell Culture

HEK293, HCT116, OVCAR8 WT cells were purchased from ATCC. I45 wild-type (WT) provided by Dr. Joseph Testa (Fox Chase Cancer Center, Philadelphia, PA). HEK293, HCT116 cells were maintained in DMEM supplemented with 10% fetal bovine serum (Invitrogen), 2 mM glutamine, 100 U/mL penicillin and 100 mg/mL streptomycin (all from Gibco). OVCAR8, JURKAT and I45 cells were maintained in complete media: RPMI 1640 with 10% fetal bovine serum (Invitrogen), 2 mM glutamine, 100 U/mL penicillin and 100 mg/mL streptomycin (all from Gibco). The cells were maintained in constant humidity level and incubated at a temperature of 37 °C. All cell lines were tested for Mycoplasma using PCR (Sigma-Aldrich, catalog no. 200-664-3) and tested negative.

### B. Lentivirus Production, Generation of Stable Cell Lines and Cell Sorting

Lentiviral transduction was utilized to create stable cell lines that expressed either eDHFR-YFP-T2A-Luciferase (eDHFR-YFP) or eDHFR-Luciferase-T2A-mCherry (eDHFR-Luc), or eDHFR-FLAG. eDHFR-Flag.eDHFR-Luc-T2A-mCherry, eDHFR-YFP-T2A-Luc and eDHFR-Flag genes were cloned into a pTRPE lentiviral vector backbone (gift of the Riley, Milone, and June labs at UPenn), and lentivirus was packaged using HEK293T/17 (ATCC) and 2nd generation packaging plasmids psPAX and pMD2 (Addgene). Target cells were transduced with lentivirus overnight in presence of 8 μg/mL of polybrene (Millipore), washed and incubated with fresh media for 1-2 days, passaged, and were sorted on either YFP (for eDHFR-YFP) or mCherry (for eDHFR-Luc), expression using fluorescence-activated cell sorting (FACS). For eDHFR-Flag-expressing cells, Hexachloroflourescien TMP is used for sorting.

C. Compound Formulation for In Vivo Studies: [¹³¹I]mI-TMP was Formulated in 2% Ethanol/Saline (v/v). The Vehicle Contained 2% Ethanol/Saline (v/v).

### D. In Vitro Assays

### (i) Preparation of HEK293T, OVCAR8, HCT116-eDHFR-Luc, I45_eDHFR_Luc for [I^{125/131}]mI-TMP Uptake

20×10⁶, HEK293T-eDHFR-Luc, OVCAR8-eDHFR-Luc and HCT116-eDHFR-Luc cells and their respective WT cells each were sedimented by centrifuging at 1200 rpm for 5 minutes. The cells were resuspended and aliquoted in 4*5×10⁶ cells/mL in RPMI (Gibco), incubated with [^{125/131}I]mI-TMP (20 E cpm, <1% ethanol) and Na[^{125/131}I]I (20 E cpm, <1% ethanol) at different time points (20, 60, 120, and 240 minutes) at room temperature. Blocked aliquots were pre-incubated with excess of cold, unlabeled trimethoprim (TMP—10 μM) and radiotracer was added after 30 minutes to determine the nonspecific binding. Following incubation, the cells were centrifuged at 1000 rpm for 8 minutes and washed with 3*5 mL of cold mixture of 1% DMSO/PBS and centrifuged again at same conditions. After the third wash, the sedimented cells were resuspended in 600 μL of PBS and split into 3 technical replicates of 200 μL. Radiotracer uptake was assayed on Gamma Counter (Perkin Elmer) with decay correction and was analyzed by dividing counts by the injected dose (ID) of [^{125/131}I]mI-TMP and Na[^{125/131}I]I and normalizing to cell number (% ID/million cells).

### (ii) Cellular Uptake Studies

20×10⁶ HCT116 eDHFR and HCT116 WT cells each were sedimented by centrifuging at 1200 rpm for 5 minutes. The cells were resuspended and aliquoted in 4*5×10⁶ cells/mL in RPMI (Gibco), incubated with [^{125/131}I]mI-TMP (1E6 cpm, <1% ethanol) and Na[^{125/131}I]I (20 E cpm, <1% ethanol) at different time points (20, 60, 120, and 240 minutes) at room temperature. Blocked aliquots were pre-incubated with excess of cold, unlabeled TMP (10 μM) and radiotracer was added after 30 minutes to determine the nonspecific binding. Following incubation, the cells were centrifuged at 1000 rpm for 8 minutes and washed with 3*5 mL of cold mixture of 1% DMSO/PBS and centrifuged again at same conditions. After the third wash, the sedimented cells were resuspended in 600 μL of PBS and split into 3 technical replicates of 200 μL. Radiotracer uptake was assayed on Gamma Counter (Perkin Elmer) with decay correction and was analyzed by dividing counts by the injected dose (ID) of [^{125/131}I]mI-TMP and Na[^{125/131}I]I and normalizing to cell number (% ID/million cells). OVCAR8, HCT116 and I45-eDHFR-FLAG cell uptakes studies were similarly performed.

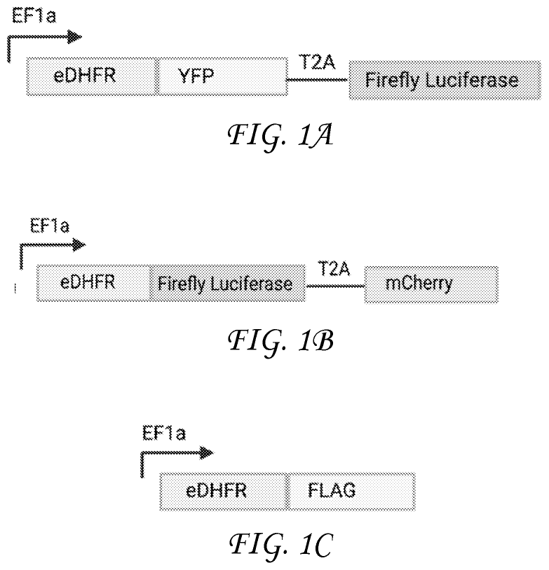

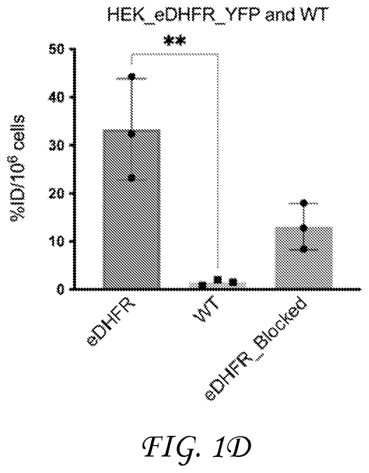

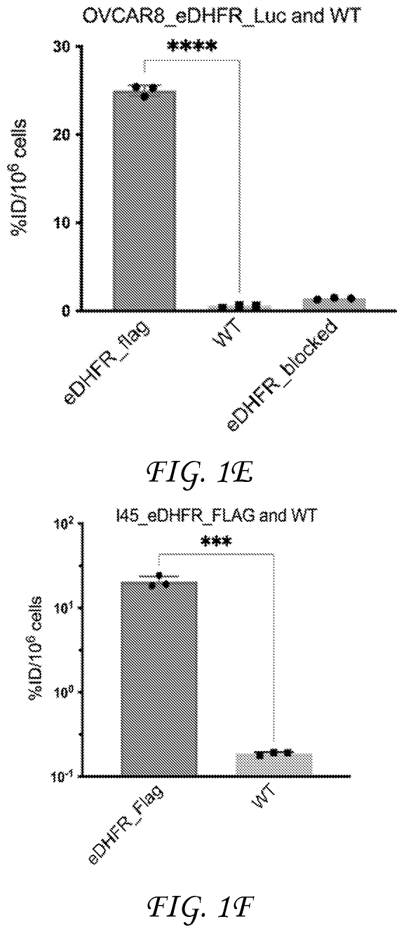

HEK 293 cells were engineered to express a fusion protein of eDHFR and yellow fluorescent protein (YFP), HEK293T-eDHFR-YFP with a C-terminal T2A firefly luciferase. Uptake studies were then conducted by incubating both HEK293T-eDHFR-YFP and HEK 293 WT cells with 0.037 MBq of [¹²⁵I]mI-TMP for 1 hour. Results showed a ~50-fold greater uptake of the radiotracer in eDHFR+ cells than in WT cells, indicating a strong uptake of the radiotracer for eDHFR+ cells. In order to evaluate the specificity of the uptake, blocked conditions were used, where eDHFR+ cells were pre-incubated with an excess of unlabeled TMP (10 μM). This resulted in a reduced uptake of compound 5b, thereby confirming the high specificity of the radiotracer for eDHFR+ cells compared to I45-wild type. See, FIG. 1D.

(iii) Washout Kinetic Assay

5×10⁶ each HCT116EDHFR and HCT116 WT cells were incubated with 20E6 cpm dose of [^{125/131}I]mI-TMP and Na[^{125/131}I]I in 15 ml conicals for 20 min at room temperature. Blocked aliquots were pre-incubated with excess of cold, unlabeled TMP (10 μM) for 30 minutes. Following 20 min incubation, the cells were washed with 3*5 mL of cold mixture of 1% DMSO/PBS. After washing, the uptake was quantified on Gamma Counter (Perkin Elmer) with decay correction and analyzed by dividing counts by the injected dose (ID) of [¹²⁵I]mI-TMP and Na[¹²⁵I]I. The cells were resuspended in 4 mL fresh media and divided into 4, 1 mL aliquots. The cells were sampled at 1 h, 2 h and 4 h. After 1 h the cells sampled at 1 h washed 3* cold mixture of 1% DMSO/PBS and uptake was assayed on gamma counter. Similarly, the process cells was repeated for 2 h and 4 h samples.

### (iv) Kill Assay

Following the same uptake protocol till washing after the third wash the cells were counted using cell counter and they were diluted to generate even number of cells/mL. In summary, 20×106 HCT116 eDHFR-Luc cells were suspended at 5×106 cells/mL and incubated with 3.7 MBq of [¹³¹I]I-TMP for 2 and 4 h at room temperature. We've applied the same blocked conditions used in the uptake assay. Following incubation, the cells were centrifuged at 1000 rpm for 8 minutes and washed with 5 mL of cold 1% DMSO/PBS three times. Then cells will be re-distributed into 96 well plates (2,000 cells/well) with fresh media to monitor cell proliferation by MTT assay on days 1, 3, 7 and 10. A total of 9 wells (3×3) were measured on the plate per sample per time point. To complete the assay cell media was removed and replaced with 50 μL of serum free media and 50 μL of MTT reagent. The plates were covered with tin foil and incubated at 37 °C for 3 hours. The plates were shaken at 200 rpm at 37 °C for 15 min. Using the microplate plate reader, sample absorbance was measured at 590 nM for 600 ms. Controls included [¹³¹I]NaI treated cells and blocking with 10 μM parent TMP (unlabeled). An analogous procedure was used for the OVCAR8-eDHFR-Luc kill assay with different doses (1.85-37 MBq) and treatment times.

### (v) Materials and Methods Information for Liver Metabolite Study

Cryopreserved primary human hepatocytes were acquired from ThermoFisher. To each well of a 96 well ultra-low attachment U-bottom plate, 2,000 cells were plated in 200 μL William's E Medium (no phenol red, 20% FBS, 1% penicillin-streptomycin, 1% insulin-transferrin-selenium solution, 0.1 M dexamethasone, 1% L-glutamine). The plate was centrifuged at 300 rpm for 2 minutes and then incubated without disturbance. After 5 days, 100 μL of spent media was removed and replaced with fresh media. On day 7, 100 μL of spent media was removed and replaced with fresh William's E Medium (no phenol red, 1% penicillin-streptomycin, 1% insulin-transferrin-selenium solution, 0.1 M dexamethasone, 1% L-glutamine) to achieve a final concentration of 10% FBS. Blocking of CYP450 activity was achieved by pre-incubation with 1-aminobenzotriazole (Cayman Chemical) at 100 μM for 4 hours prior to the addition of 1 μCi (0.037 MBq) [¹³¹]mI-TMP. Samples were collected after 2 and 24 hours by transfer of the entire well volume into an Eppendorf tube and centrifugation for 10 minutes at 13,000 rpm. The supernatant was then removed and analyzed on an Agilent 1260 Infinity II LC system equipped with an Eckert & Ziegler FlowCountPRO detector.

### (vi) Hepatocytes Spheroid Assay

Specifically, 2,000 cells were plated in 200 μL William's E Medium (no phenol red, 20% FBS, 1% penicillin-streptomycin, 1% insulin-transferrin-selenium solution, 0.1 M dexamethasone, 1% L-glutamine). Blocking of CYP450 activity was achieved by pre-incubation with 1-aminobenzotriazole (Cayman Chemical) at 100 μM for 4 hours prior to the addition of 1 μCi [¹³¹]mI-TMP. Samples were collected after 2 and 24 hours by transfer of the entire well volume into an Eppendorf tube and centrifugation for 10 minutes at 13,000 rpm. The supernatant was then removed and analyzed on an Agilent 1260 Infinity II LC system equipped with an Eckert & Ziegler FlowCountPRO detector.

Specifically, cryopreserved hepatocytes were thawed and diluted 1:10 in a 15 mL conical tube with warm culture media (William's E Medium, no phenol red, 20% FBS, 1% penicillin-streptomycin, 1% insulin-transferrin-selenium solution, 0.1 M dexamethasone, 1% L-glutamine). Supernatant was aspirated and resuspended in 5 mL culture media for counting. Cells were counted and diluted to a concentration of 10,000 live cells/mL. 200 μL of the cell suspension was plated into each well of a 96-well ultra-low attachment U-bottom plate. The plate was spun at 300 rpm for 2 minutes and set in an incubator that sees minimal use and leave undisturbed for 5 days. After 5 days, 100 μL of media from each well was removed and replaced with 100 μL of fresh media. On day 7, 100 μL of media from each well was removed and replaced with 100 μL of fresh media without FBS. This set the FBS concentration of the assay media at 10%. For blocking of CYP450 activity, 1-aminobenzotriazole was added to a concentration of 100 M and incubated for 4 hours. One μCi radioligand was added to each well. At desired time points, the entire volume of the well was transferred into an Eppendorf tube and centrifuge at 13,000 rpm for 10 minutes. The supernatant was transferred into an autosampler vial and analyzed by HPLC.

(vii) Serum Stability

In vitro serum stability of was performed by adding the [¹³¹I]mI-TMP (50 μL, 1.85 MBq) to fetal bovine serum (500 μL). The mixture was then incubated in a ThermoMixer (800 rpm) at 37 °C, and deiodination was analyzed by radio-TLC (10% NH40Ac:MeOH=3:7) at 0, 24, and 72 h. In this condition, free I-131 moved to solvent front whereas intact [¹³¹I]I-TMP remained at the origin.

The serum stability experiment demonstrated the stability of radio tracer 5b in FBS at 37 °C for 72 hours without any detectable deiodination confirming the stability of 5b in serum. See, FIGS. 8A-8C.

(viii) Specific Activity Measurement

Molar activity (Am) was calculated by applying the formula:

$$Am = \frac{\% radiochemical purities}{number of moles of the non - radioactive compound} \times radioactivity$$

The number of moles of the non-radioactive compound was established by integrating the corresponding UV peak area from the reversed-phase high-performance liquid chromatography (RPHPLC) using a calibration curve.

A calibration curve was first prepared by injecting various amounts (10 ng-10 g) of non-radioactive TMP-I into HPLC (λₘₐₓ=288 nm). Subsequently, a known amount of activity of the purified [¹³¹I]I-TMP (1 MBq) was injected into HPLC, and the peak area obtained in the UV chromatogram was used to calculate specific activity and was found to be 148 GBq/mol.

The HPLC-detected radioactivity was recorded and expressed in gigabecquerel (GBq), while the number of moles of the compound was expressed in micromole (mol). The calibration curve was determined for different concentrations of [¹³¹I]mI-TMP ranging from 0.01 to 15 g were analyzed on RP-HPLC at 254 nm.

The HPLC system (5 m, 4.6×150 mm) was used. Elution was performed at a flow rate of 1 mL/min using the following gradient program: 10% B from 5 to 15 min, 10-90% of B from 15 to 20 min, 90% B from 20 to 30 min, and 90-0% B from 30 to 40 min (mobile phase A: 0.1% trifluoroacetic acid (TFA) in water; mobile phase B 0.1% TFA in acetonitrile). The retention times of [¹¹³I]mI-TMP was 10.3.

### (ix) Determination of Partition Coefficient

Following the radio synthesis, [¹³¹I]mI-TMP (5b) was evaluated for its specific activity, lipophilicity, and serum stability. Log D values of compound 5b were determined by employing the octanol/PBS method. In this method, a mixture of n-octanol and phosphate buffered saline (pH 7.4) (1:1 v/v, 1 mL) was prepared, and 5 μL of the tracer (1.85 MBq) was added to it. The mixture was vigorously vortexed for 5 min at room temperature and then centrifuged (10000 rpm, 5 min) to ensure complete separation of both layers. After that, 100 μL aliquots were taken from each phase and counted separately using a γ-counter. The partition coefficient was calculated as the ratio of counts in 1-octanol to that in PBS. Log D values were measured six times and reported the average as −1.66 which indicates sufficient hydrophilicity for distribution to target eDHFR engineered tissues. The reported Log D_{7.4} value is the average of six readings. An in vitro hepatocyte culture study showed minor de-iodination after 24 hours, which could be inhibited with the non-specific CYP450 inhibitor 1-aminobenzotriazole (FIG. 14).

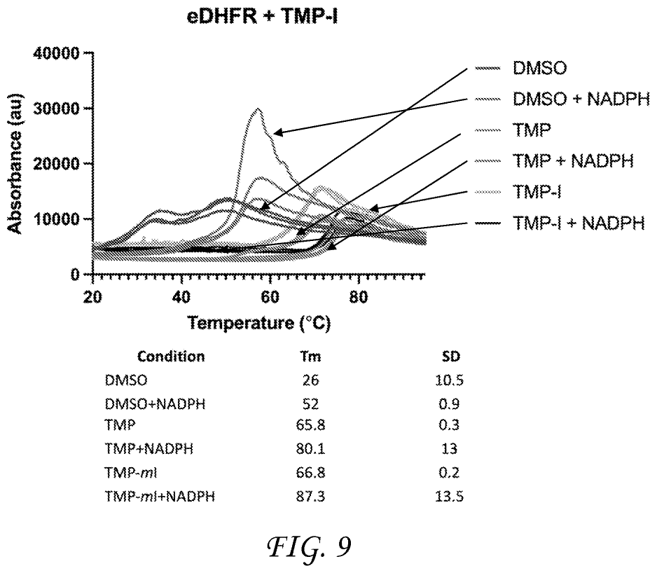

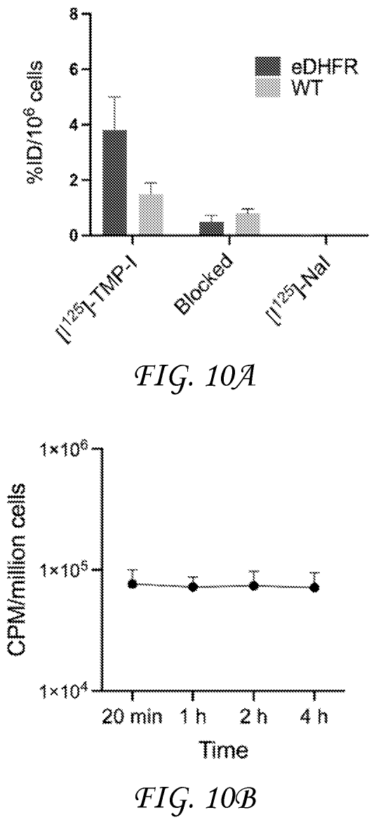

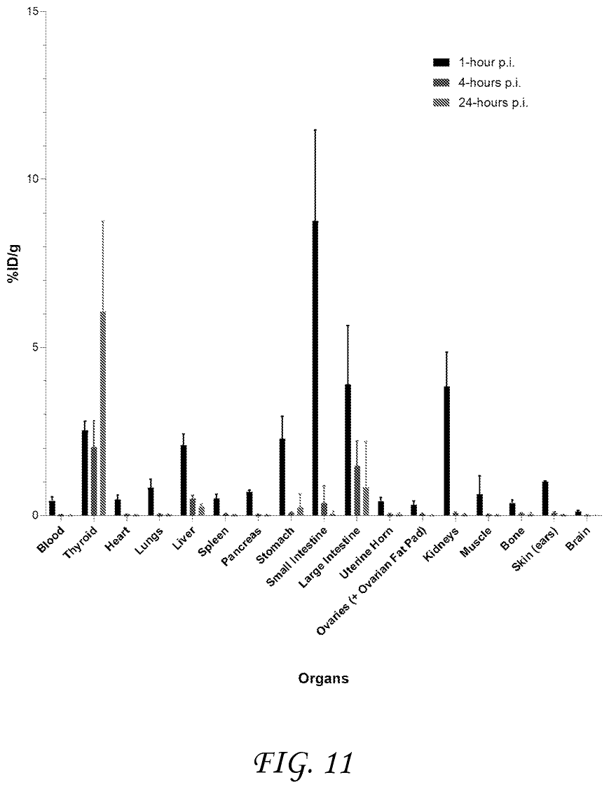

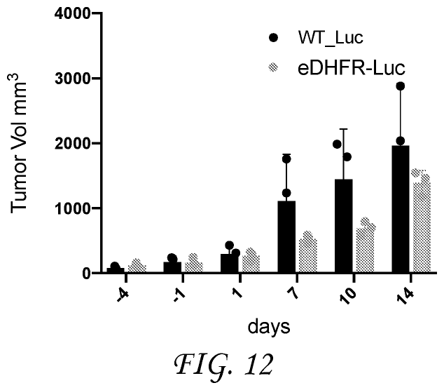

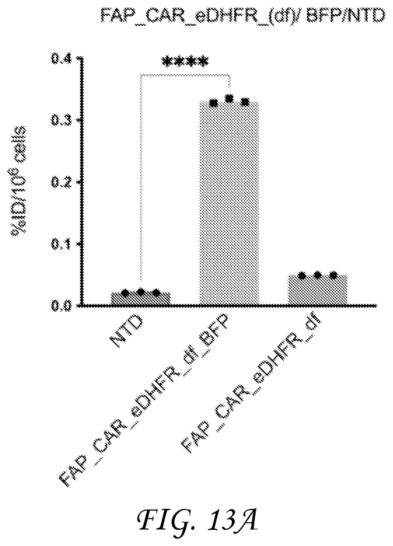

The binding affinity of TMP-I (3b) to eDHFR was evaluated by thermal shift assay, which reveals that 3b strongly stabilizes eDHFR, similar to TMP (FIG. 9). Based on the above findings, the radioiodinated TMP 5b was tested in biological systems.

### (x) Differential Scanning Fluorimetry (DSF)

Purified protein is diluted to 0.1 mg/mL protein in 500 μL in mild buffer, 25 mM HEPES pH=7.5, 200-500 mM NaCl, DTT. 17 μL of the 0.1 mg/mL protein sample is aliquoted into an opaque white 384 well PCR plate. Then 2 μL of stabilizing additive or 1 μL additive and 1 μL of concentrated drug (10 mM TMP or 10 mM cold mI-TMP or vehicle, plus 1 μL of 1×SYPRO Orange dye was added and the total well volume is 20 μL. The plate is sealed with clear optical cover, protect it from light with foil, and let it incubate for 5 minutes. Then the plate is centrifuged at 3,600×g for 2 minutes. The plate is analyzed on RT-PCR machine using a temperature-ramp method that ranges from 20-95 °C.

### (xi) Uptake Studies

To investigate the uptake of [^{125/131}I]mI-TMP, different cell lines were engineered to express eDHFR. HEK 293 cells were engineered to express a fusion protein of eDHFR and yellow fluorescent protein (YFP), HEK293T-eDHFR-YFP with a C-terminal T2A firefly luciferase. HCT116 and OVCAR 8 cells were engineered to express fusion protein eDHFR-firefly luciferase (eDHFR-Luc), HCT116-eDHFR-Luc and OVCAR 8-eDHFR-Luc with C-terminal T2A mCherry fluorescent protein. I45 cells were transduced to express eDHFR-FLAG. See, FIGS. 1A-1C.

HEK293T-eDHFR-YFP and HEK 293 WT cells were incubated with 0.037 MBq of [¹²⁵I]mI-TMP for 1 hour and showed a ~30-fold increased uptake of the radiotracer in eDHFR+ cells than in WT cells. In order to evaluate the specificity of the uptake, blocked conditions were used, where eDHFR+ cells were pre-incubated with an excess of unlabeled TMP (10 μM) for 20 min. This resulted in a reduced uptake of compound 5b, confirming the specificity of the radiotracer for eDHFR+ cells. See, FIG. 1D.

After observing the uptake of the radiotracer [¹²⁵I]mI-TMP in HEK-eDHFR cells, the internalization of [^{125/131}I]mI-TMP in tumor cell lines was investigated. In initial uptake studies in tumor cell lines, OVCAR WT and OVCAR 8-eDHFR-Luc cells were exposed to 0.037 MBq of [¹³¹I]mI-TMP for 1 hour. Consistent with our findings in HEK cells, a ~25-fold higher tracer uptake was observed in OVCAR8-eDHFR-Luc+ cells compared to OVCAR8 WT cells, demonstrating the high specificity of [¹³¹I]mI-TMP tracer for eDHFR. Again, blocked conditions, by preincubating the eDHFR+ cells with an excess of TMP, resulted in reduced uptake of radiotracer 5b in OVCAR8 eDHFR cells. See, FIG. 1E. The radiosensitivity of [¹³¹I]mI-TMP in mesothelioma cells I45 cells was then analyzed. An ~80-fold uptake in eDHFR+ cells compared to wild type was observed. These findings show that the radiotracer as therapeutic efficacy against various types of tumors. See, FIG. 1F.

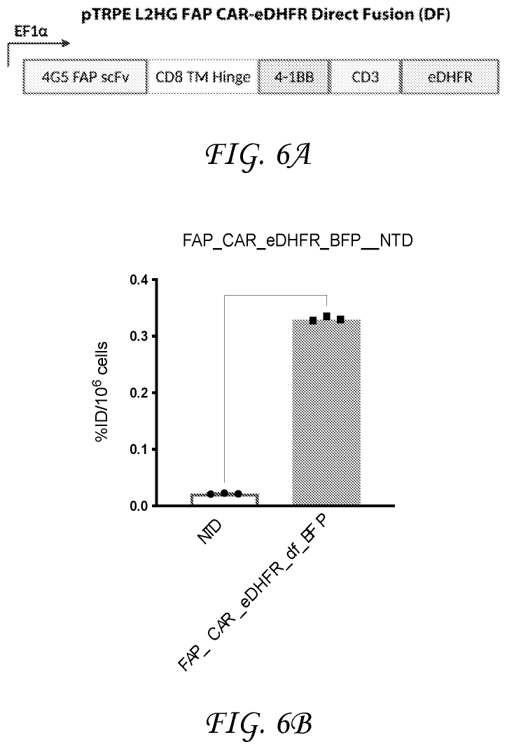

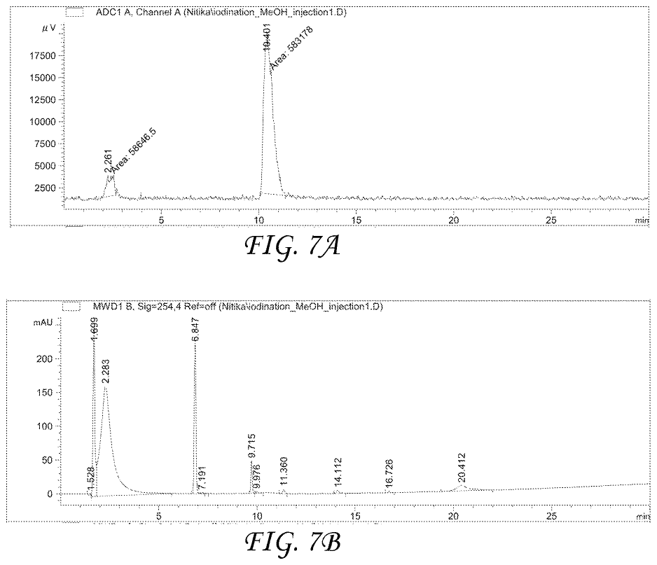

Next, the uptake radio-iodinated probe 5b was evaluated in primary human CAR-T cells. CAR-T cells were engineered to express eDHFR which is fused directly to the C-terminus of the signaling CD3zeta domain of the fibroblast activation protein (FAP) CAR. The FAP-CAR-eDHFR cells (3×10⁶) and NTD cells were incubated with 0.074 MBq of [¹³¹I]mI-TMP, incubated for 60 min at 37 °C. After incubation, the cells were centrifuged at 1200 rpm and were washed thrice with cold PBS (Corning). Subsequently, the cell pellet was resuspended in 1% DMSO/PBS and divided into 3 technical replicates of 100 μL each. The uptake of radiotracer was quantified using a gamma counter (PerkinElmer). The final uptake was expressed as a ratio of % ID normalized per 106 cells (% ID/million cells). An about 15 fold more uptake in eDHFR+ cells was observed as compared to NTD cells. See, FIGS. 6A and 6B. The specificity is further assessed by comparing with FAP_CAR_eDHFR(df) which has low eDHFR expression. Less uptake of radiotracer 5b was observed as compared to high expressing eDHFR FAP_CARs. Also, the washout study to check the retention of the radiotracer in FAP_CAR_eDHFR cells showed strong internalization of compound 5b even after 5 h.

(xii) Therapeutic Efficacy

To investigate the therapeutic potential of m[¹³¹I]mI-TMP 5b in tumor cell lines, OVCAR8 eDHFR+ cells were exposed to varying doses (ranging from 1.85-37 MBq) of the radiotracer of 5b at different incubation times. Different conditions were employed to assess the selective therapeutic effect of the radio tracer on eDHFR+ cells. To determine the specific killing of eDHFR+ tumor cells, blocked conditions were analyzed. The cell viability of compound 5b-treated cells was also compared with the cells exposed to [¹³¹I]I—Na. Additionally, untreated cells were used as controls. The MTT assay was used to measure the cell viability of the cells under the aforementioned conditions on days 1, 3, 7, and 12.

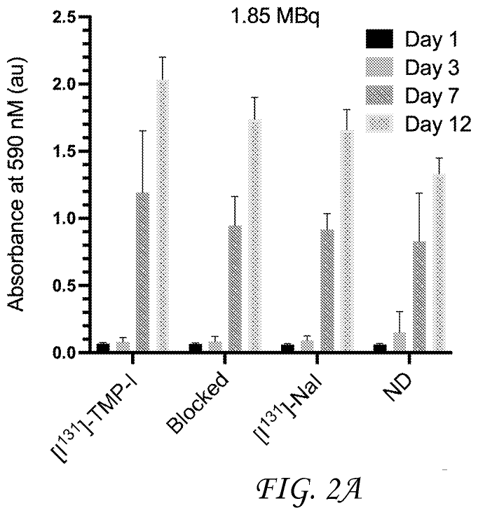

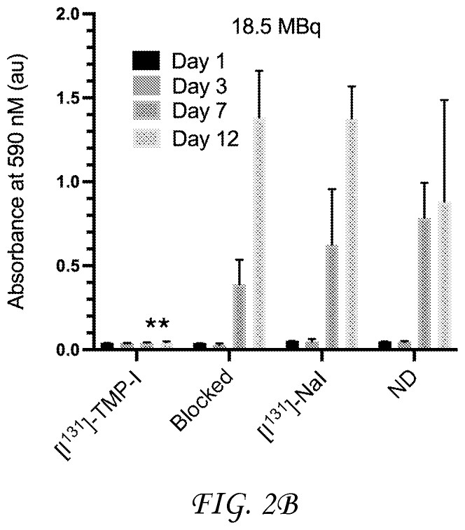

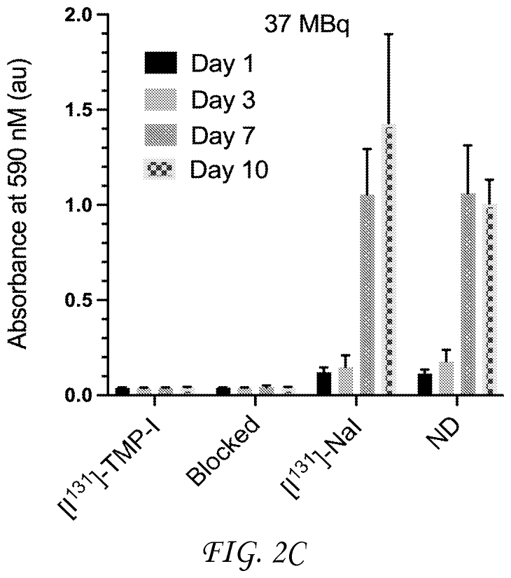

OVCAR8-eDHFR-Luc+ cells were treated with 1.85 and 18.5 MBq of m[¹³¹I]mI-TMP 5b and [¹³¹I]I—Na, incubated for 1 hour. The cells received lower dose of compound 5b (1.85 MBq) did not exhibit any cytotoxic effects/decreased viability. The cell viability of the compound 5b-treated cells was similar to cells under blocked, [¹³¹I]NaI, and no drug conditions. See, FIG. 2A. However, the eDHFR+ cells treated with 10× higher dose of compound 5b (18.5 MBq) for 1 h, showed strong cytotoxic effects, and thus no cell regrowth/significantly lower viability was observed at day 7 and even at day 12 (indicated by **). In contrast, cells treated with [¹³¹I]I—Na showed viability and significant growth. The cells treated with the blocking agent exhibited comparable viability to those under no treatment or [¹³¹I]I—Na conditions. See, FIG. 2B. Based on the results obtained, a continued dose escalation was considered to better understand the therapeutic window. Finally, OVCAR_eDHFR+ cells were tested with 37 MBq dose of m[¹³¹I]mI-TMP. However, this resulted in non-specific cell killing, with eDHFR+ cells under blocked conditions behaving the same way as eDHFR+ cells only treated with compound 5b (FIG. 2C), which occurred even when decreasing the incubation time to 20 minutes.

Next, the therapeutic effect of [^{131/125}I]mI-TMP in a human colorectal carcinoma cell line, HCT116 was analyzed. Results showed two-fold uptake of radiotracer in eDHFR+ cells in comparison to their wild-type counterparts, which was in contrast to the robust uptake in CAR T cells, I45 and HEK293 previously. The specificity of radiotracer uptake was determined by blocking conditions. TMP competed with [^{131/125}I]mI-TMP and resulted in reduced uptake of the radiotracer by eDHFR+ cells. See, FIG. 10A.

A washout study was performed to know the retention of the tracer in the cells. HCT116_eDHFR cells were incubated with 0.037 MBq of compound 5b for 20 minutes. The cells were washed, resuspended and sampled at 60, 120 and 240 minutes. The result demonstrates substantial retention of [¹³¹I]mI-TMP in eDHFR+ cells even after 4 hours. To examine the radiotherapeutic effect, the incubation period was increased and the dose decreased. The cells were dosed with 3.7 MBq at 37 °C and incubated them with 2 and 4 hours. See, FIG. 10B.

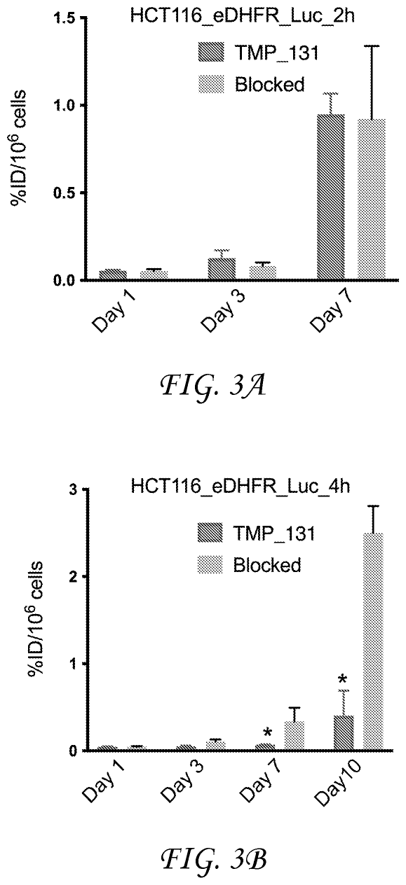

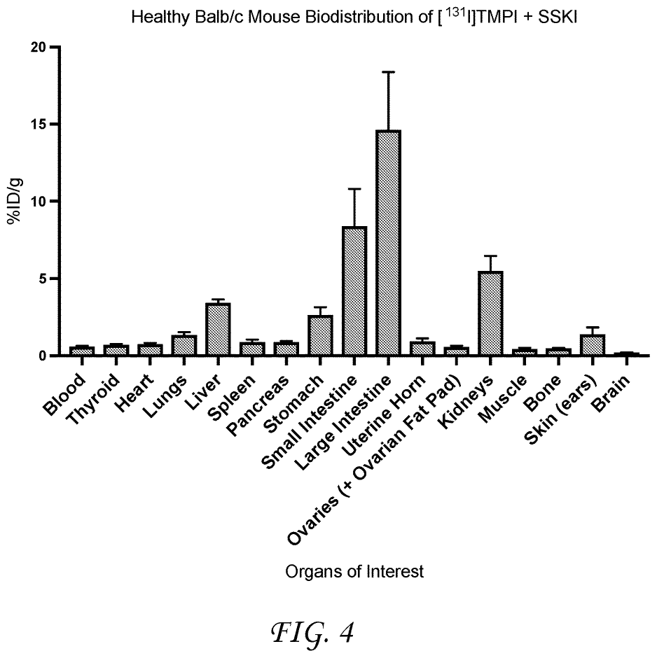

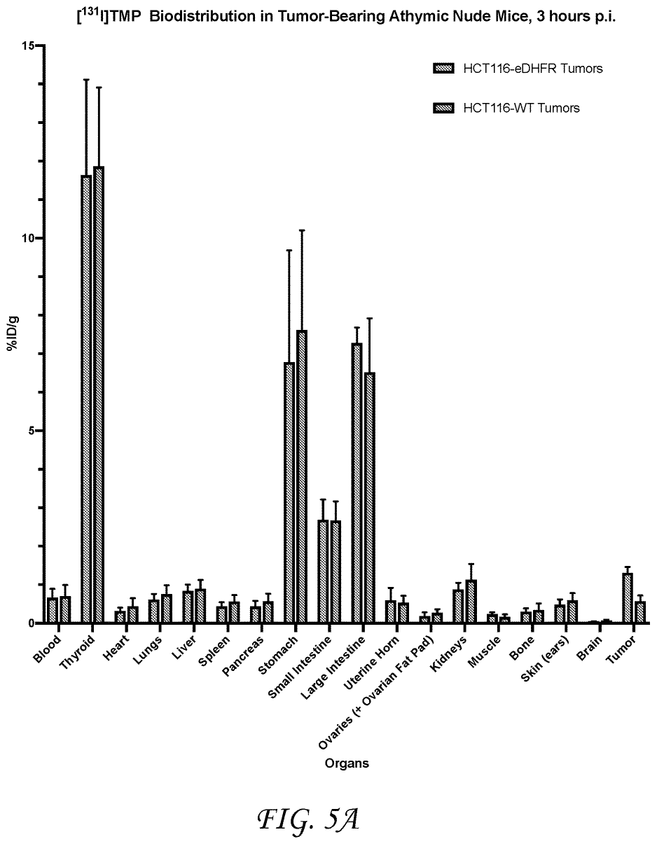

Despite lower uptake in HCT116 cells, cell viability was measured on days 1, 3, 7, and 10 using the MTT assay and compared with blocked, no drug and [¹³¹I]I—Na treated cells. Cells incubated for 2 hours did not exhibit any cytotoxic effect. See, FIG. 3A. However, the cells incubated for a longer period, 4 hours, demonstrated a cytotoxic effect (indicated by *), decreased viability and a therapeutic window compared to blocked cells. Thus, the cells receiving only compound 5b did not show any viability on day 7 and exhibiting mild growth at day 10 in comparison to the blocked conditions. See, FIG. 3B.

Tumor size measurements were taken using calipers from day 1 to 14 post-injection of the [¹³¹I]mI-TMP to monitor the response of radiation therapy. Analysis of the data revealed that mice with eDHFR-Luc tumors showed a reduction in tumor size at day 7 post-injection, which remained stable until day 10 but increased by day 14. Conversely, eDHFR-negative tumors continued to grow from day 7 to day 14, illustrates the specificity and effectiveness of [¹³¹I]mI-TMP for eDHFR-positive tumors. These results are consistent with in vitro data, which showed a two-fold increase in uptake in eDHFR+ HCT116 cells compared to WT cells. Furthermore, the cell viability assay revealed no cell regrowth at day 7, but mild regrowth was observed at day 10 and beyond. See, FIG. 11 and FIG. 12.

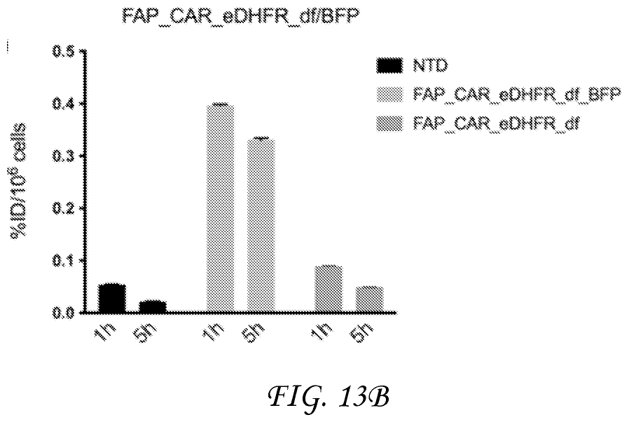

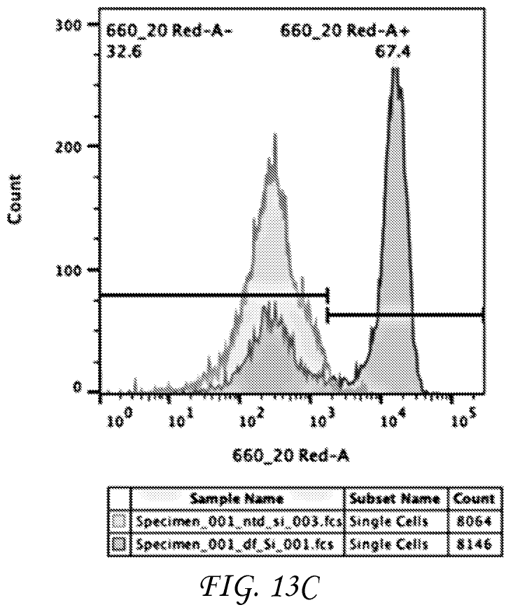

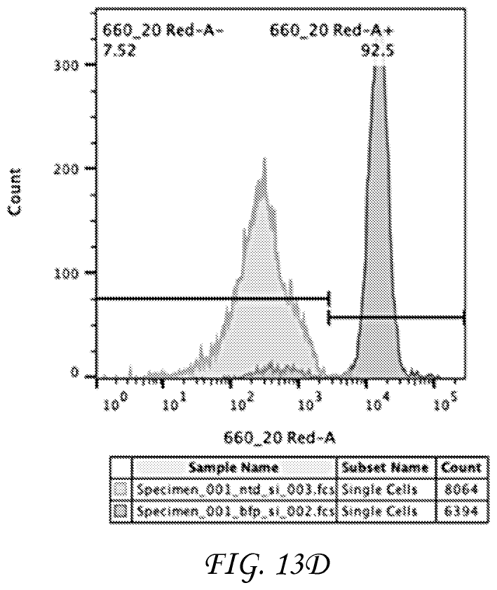

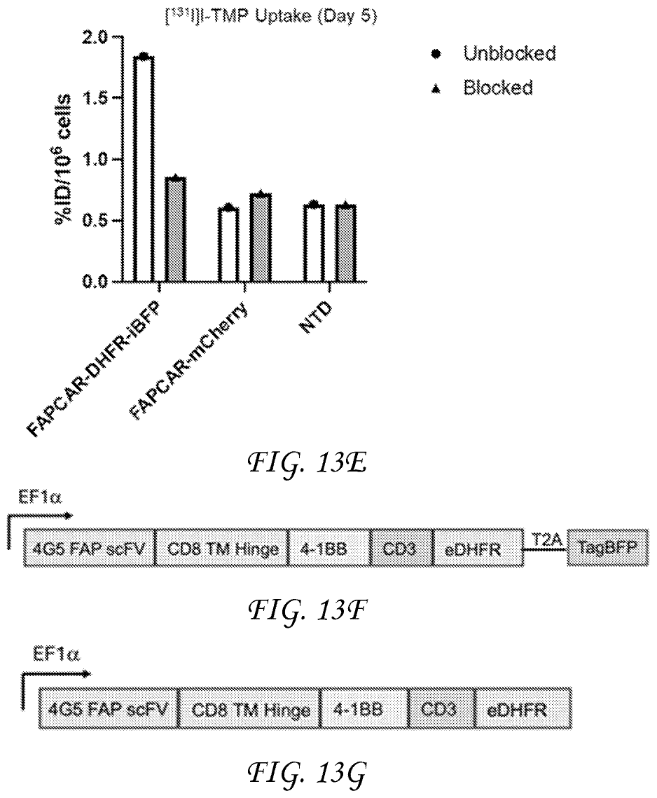

RLT in I45 tumors. Given that there was more robust uptake of [¹³¹I]I-TMP in I45 eDHFR-FLAG cells (FIG. 1F), it was then tested whether this would translate in vivo and again tested for cytotoxicity. Briefly, I45 eDHFR-FLAG and WT cells were xenografted into the shoulders of female athymic CD1-Foxn1nu mice. The tumors were allowed to grow for ~2 weeks. Subsequently, [¹³¹I]I-TMP was administered intravenously and a limited biodistribution study was performed. [¹³¹I]I-TMP was present in eDHFR+ tumors, even after 24 hours (FIG. 30A). The dynamic range of uptake showing over 1000-fold increased at 24 hours in eDHFR tumors compared to WT tumors (FIG. 30B), without substantial washout. To assess for cytotoxicity in I45 mesothelioma cells, I45 WT and I45 eDHFR tumors were separately xenografted into CD1-Foxn1nu mice and either [¹³¹I]I-TMP or saline was administered to form 4 experimental cohorts (n=7). A clear reduction in tumor growth was seen in I45 eDHFR tumors, while not in any other of the controls (FIGS. 31A and 31B).

[¹³¹I]I-TMP in CAR T cells. Finally, the in vitro uptake of [¹³¹I]I-TMP was evaluated in primary human CAR T cells as a model system that could potentially be used to localize RLT protein targets, like eDHFR, to tumors. In this case, two different fibroblast activation protein (FAP) CAR T cell constructs that express eDHFR fused directly to the C-terminus of the signaling CD3zeta domain of the CAR were used (7) (FIGS. 13F and 13G). The FAP-CAR-eDHFR T cells and NTD T cells were treated with 0.074 MBq of [¹³¹I]I-TMP and incubated for 1 hour at 37 °C. There was ~15-fold more uptake in FAP-CAR-eDHFR-DF-BFP in comparison to NTD cells at day 8 of T cell expansion (FIG. 13A). A washout study was performed again to check the retention of the radiotracer in FAP CAR-eDHFR cells which showed retention of [¹³¹I]I-TMP even after 5 hours (FIG. 13B). A follow up study at an earlier day of expansion showed ~3-fold higher uptake, suggesting that the timing of CAR T cell expansion and protein expression may play a role in radiotracer uptake (FIG. 13E).

### Example 3: In Vivo Studies

### (i) Healthy Mouse Biodistribution Study

Given promising in vitro experimental results, a biodistribution study was conducted on healthy balb/c mice. 8-12-week-old female balb/c mice (n=9) were administered with 0.74 MBq of [¹³¹I]mI-TMP via the tail vein. Mice were then euthanized 1-hour, 4-hour, and 24-hour post radiotracer injection, and organs were harvested for bio-distribution quantification. Ex-vivo biodistribution studies were conducted at 1-, 4-, and 24-hours post-injection of [¹³¹I]mI-TMP to assess the uptake of the tracer accumulation in different organs or tissues. Total radioactive uptake per organ, in CPM, was determined by γ-counter (Wizard Detector, Perkin Elmer). After 1 h time point, there was a significant increase in the radioactivity in the kidney, small intestine, stomach, and liver. However, over time, 24 h ex-vivo biodistribution study showed an increase in radioactivity in thyroid, indicating the deiodination of [¹³¹I]mI-TMP. Minimal to no uptake was observed in the heart, lungs, brain, pancreas, bones and spleen at all time points. See, FIG. 10A-10B and FIG. 11. These tissues could be sites where eDHFR could be localized using genetic vectors to achieve a high therapeutic index.

Another ex-vivo biodistribution study was performed with potassium iodide (SSKI) administration 8-12-week-old female balb/c mice (n=9) were administered with intraperitoneal administration for thyroid protection, with a dose of 100 mg/kg potassium iodide in a volume of 100 μL/10 g given the day before treatment, 30 minutes before the radioactive [¹³¹I]mI-TMP injection. The mice (n=4) were administered 0.67 MBq of [¹³¹I]mI-TMP via the tail vein. Ex-vivo biodistribution studies were conducted at 1-hour post-injection of [¹³¹I]mI-TMP to assess the uptake of the tracer accumulation in different organs or tissues. Minimal to no uptake was observed in the heart, lungs, brain, pancreas, bones and spleen (FIG. 4). KI pre-treatment abrogated thyroid uptake, and again, there was a significant increase in the radioactivity in the kidney, small intestine and large intestine while the stomach and liver showed low uptake, and the blood, heart, lungs, brain, pancreas, bones and spleen had negligible uptake (FIG. 4).

(ii) In vivo HCT116 Tumor Model

To evaluate the therapeutic potential [¹³¹I]mI-TMP, sixteen (16) 8- to 12-week-old female athymic nude mice (Charles River Laboratories) were inoculated with 1×10⁷ HCT116 tumor cells (eDHFR+ or WT) on the left shoulder. The mice were then divided into two groups: right (8) mice with HCT116-eDHFR (eDHFR-positive cells) and Eight (8) mice with HCT116-WT (eDHFR-negative) cells.

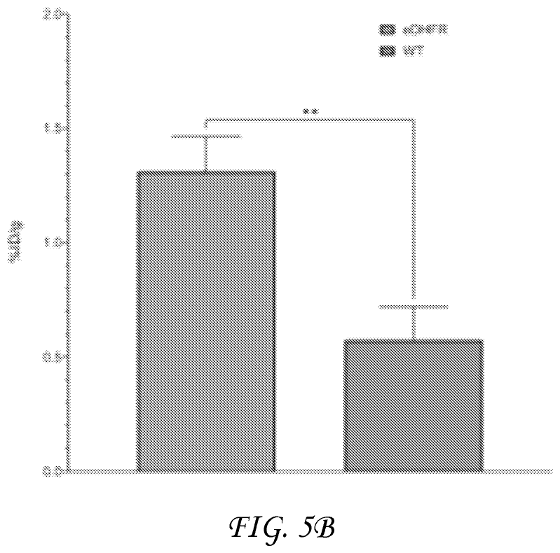

Ex-vivo biodistribution studies were conducted on three (3) tumor-bearing mice from each group, while the remaining ten (10) mice were used to monitor tumor response to radiotherapy. The ex-vivo biodistribution studies showed that [¹³¹I]mI-TMP was taken up twice as much in tumors with eDHFR cells than in tumors without eDHFR cells (WT) (FIGS. 5A and 5B), which mirrored the uptake limits of our in vitro HCT116 data (FIG. 10A).

(iii) HCT116 Tumor Model Biodistribution Study

On day 13 post tumor inoculation, three (3) HCT116-eDHFR and three (3) HCT116-WT tumor-bearing athymic nude mice were injected with 50 μCi/1.85 MBq of [¹³¹I]mI-TMP via tail vein. Three (3) hours post radiotracer injection mice were euthanized, and organs of interest were harvested for biodistribution quantification. Total radioactive uptake per organ, in counts per minute (CPM), was determined by γ-counter (Perkin Elmer).

### (iv) [¹³¹I]mI-TMP Radiotherapy Administration, Imaging, and Monitoring

On day 13 post tumor inoculation, five (5) HCT116-eDHFR and five (5) HCT116-WT tumor-bearing athymic nude mice were injected with a radiotherapy dose of 500 μCi of [¹³¹I]mI-TMP via tail vein. Three (3) of the five (5) mice from each cohort were imaged at 3- and 24-hours post [¹³¹I]mI-TMPI radiotherapy injection using small animal SPECT and CT instruments. Five (5) mice from each cohort (which included the 3 imaged mice from each cohort) were then monitored for 14 days using caliper measurements and IVIS imaging.

(v) In vivo I45 eDHFR-FLAG Tumor Model. Female CD1 nu/nu mice, aged six to eight weeks, were subject to subcutaneous xenografting. Specifically, I45 eDHFR-Flag cells (1×10⁶ cells/mouse) were suspended in a solution comprising equal volumes of 75 μL RPMI complete medium and 75 μL Matrigel Matrix (Corning, 354234) for implantation on the shoulder.

### (vi) I45 RLT

[¹³¹I]I-TMPI Administration, and Monitoring. Female CD1 nu/nu mice, aged six to eight weeks, underwent subcutaneous xenografting with 1×10⁶ I45 WT and I45 eDHFR-Flag cells on the shoulder. The cells were suspended in a 1:1 volume ratio of 75 μL RPMI complete medium and 75 μL Matrigel Matrix (Corning, 354234). Both I45 WT and I45 eDHFR-Flag mice were randomly divided into two groups, with each group consisting of 7 mice. Tumors were grown for 2 weeks. Prior to the injection of [³¹I]I-TMP (18.5 MBq/mouse), a solution of potassium iodide in saline (2 mg/200 μL) was administered intraperitoneally to each mouse, both 24 hours and 1 hour before the injection of [¹³¹I]I-TMP. [¹³¹I]I-TMP, diluted in 100 μL saline, was injected into two groups containing I45 WT and I45 eDHFR-Flag xenografts on day 9 of initial tumor inoculation. The remaining two groups of I45 WT and I45 eDHFR-Flag xenografts were injected with saline. Tumor growth was monitored from two days before to 10 days after [¹³¹I]I-TMP injection, with measurements taken every other day using calipers. Subsequently, the animals were euthanized under anesthesia, following the guidelines of the University of Pennsylvania.

### Example 4

This example illustrates the favorability of radiolabeling the meta position over the para position. EC₅₀ dose response curves for eDHFR binding were generated as described in Iwamoto, Chemistry & Biology, 17(9):981-988, 2010 for TMP derivatives in HCT-116 cells which constitutively express a tsLuc-DD-DHFR fusion protein. TMP-m-Br and TMP-m-I, neither radiolabeled, show similar EC₅₀ values (5 nM) to parent TMP (5 nM). TMP-Bn-I (para-iodo benzyl at the para position on the TMP trimethoxy benzene ring) shows a large reduction in EC₅₀ (1970 nM) compared to the smaller meta-TMP derivatives. See, FIG. 15. Specifically, this data shows that the meta-position is favored compared to the TMP-Bn-I at the para position.

### Example 5

Using standard cloning procedures and DNA sequence synthesis, the recombinant eDHFR protein was modified in the following ways. First, the eDHFR nucleotide sequence was extended with a yellow fluorescent protein (YFP) domain at the C-terminus to construct a fusion eDHFR-YFP protein. See, Sellmyer, Mol Ther 2020; 28:42-51. Then, one of the following localization domains was added: simian virus 40 nuclear localization signal (NLS) at the N-terminus, human immunodeficiency virus Rev protein nuclear export signal (NES) at the C-terminus, and Saccharolobus solfataricus P2 DNA-binding domain 7d (DNAB) at the N-terminus. See, Kobayashi, Antimicrob Agents Chemother 2006; 50:1118-1119; and Kalichuk, Sci Rep 2016; 6:37274. The resulting library of pUC57 plasmids, each containing one of NLS-eDHFR-YFP, eDHFR-YFP-NES, and DNAB-eDHFR-YFP, were successfully cloned into a lentiviral vector. The lentiviral plasmids were introduced into HEK293T cells for viral production and isolation.

Then, three human cancer cell lines were cultured: HCT116 (colorectal cancer), IMR5 (neuroblastoma), and SKOV3 (ovarian cancer). They were transfected with the three lentiviral constructs for expression of NLS-eDHFR-YFP, eDHFR-YFP-NES, and DNAB-eDHFR-YFP, resulting in nine cell lines in total. Subcellular localization of eDHFR-YFP in the cell lines was verified with fluorescence microscopy. The eDHFR-YFP-NES and DNAB-eDHFR-YFP showed excellent localization to the cytoplasm and DNA, respectively, while the NLS-eDHFR-YFP showed predominant but non-exclusive nuclear localization.

Astatine-211 was produced at the in-house cyclotron with radionuclidic purity of over 99.9%, as described in Batra, Clin Cancer Res 2022; 28:4146-4157. Then, ²¹¹At-para-astatobenzyl-trimethoprim (²¹¹At-PAB-TMP) with over 95% radiochemical purity was prepared by from the 5-(3,5-dimethoxy-4-((4-(4,4,5,5-tetramethyl-1,3,2-dioxaborolan-2-yl)benzyl)oxy)benzyl)pyrimidine-2,4-diamine (BPIN precursor).

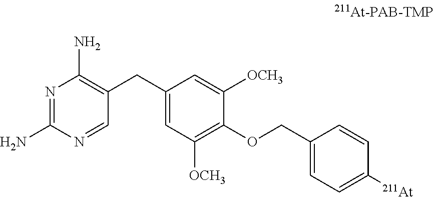

Cytotoxicity experiments were performed as follows: wild-type IMR5 cells as well as those expressing eDHFR-YFP localized to the DNA, nucleus, and cytoplasm were treated with various concentrations of ²¹¹At-PAB-TMP for 72 hours. The survival fraction for each treatment condition was measured with luminescent cell viability assay.

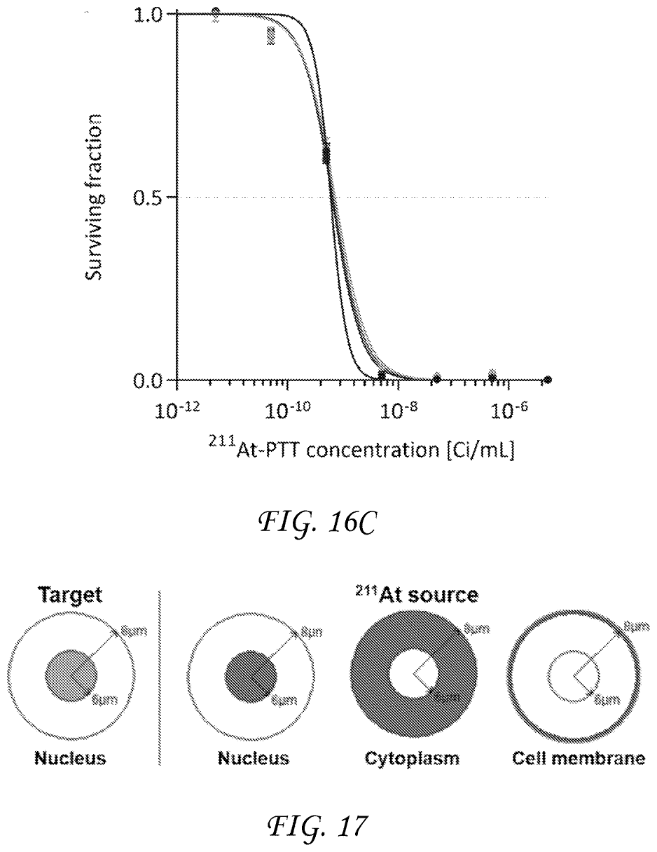

The resulting survival vs. dose curves were analyzed to determine effective concentrations for 50% reduction in cell viability (EC₅₀). See, FIGS. 16A-16C. As anticipated, cytotoxicity of ²¹¹At-PAB-TMP was the highest in the IMR5 cells expressing DNA-bound eDHFR-YFP, followed by nuclear and cytoplasmic eDHFR-YFP. See, FIGS. 16A-16C. As a positive control, treatment with ²¹¹At-PTT that targets poly(ADP-ribose) polymerase 1 in the cell nucleus did not result in difference in cytotoxicity among the IMR5 cells (FIG. 16C, See, Makvandi, Mol Cancer Ther 2019; 18:1195-1204), illustrating that the difference in cell survival seen with ²¹¹At-PAB-TMP is attributed to subcellular targeting of eDHFR rather than difference in radiosensitivity of the different cell lines in general. ²¹¹At-PTT refers to astatine-211-parthanatine and is also known in the art as [²¹¹At]MM4. ²¹¹At-PTT has the following structure and may be prepared as described in Reilly, Org Lett. 2018 Apr. 6; 20(7): 1752-1755.

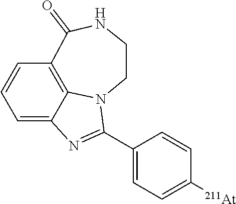

### Example 7

Preliminary Monte Carlo dosimetric calculations with MIRDcell v3 was performed to calculate the nuclear dose deposition from ²¹¹At decay. Using the geometric dimensions of IMR5 cells (Lee, J Nucl Med 2020; 61:850-856), the absorbed dose to the cell nucleus was 6.01 cGy/Bq·s, 2.48 cGy/Bq·s, and 1.78 cGy/Bq·s per each ²¹¹At decay event in the nucleus, cytoplasm, and plasma membrane, respectively. See, FIG. 17. The absorbed dose to the cell nucleus was the highest with ²¹¹At in the cell nucleus, followed by the cytoplasm and the cell membrane.

### Example 8

### A. Chemistry and Radiolabeling

Astatine-211 was produced at the University of Pennsylvania Cyclotron Facility. [²¹¹At]At-TMP was synthesized from meta-iodo-TMP via a stannane precursor as outlined in Scheme 2 as described herein.

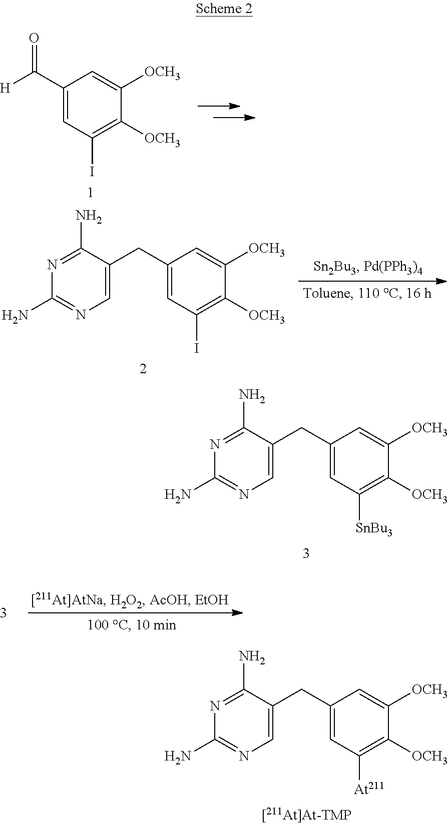

Radio-HPLC was used to isolate [²¹¹At]At-TMP from unreacted free ²¹¹At and other byproducts with >95% chemical and radiochemical purity. The isolated [²¹¹At]At-TMP was filtered through a Sep-Pak C18 Cartridge (Waters) and eluted in ethanol.

The specific activity of [²¹¹At]At-TMP and [¹²⁵I]I-TMP was determined by comparison of radioactivity to the area under 288-nm ultraviolet light peak on radio-HPLC. The in vitro stability of [²¹¹At]At-TMP was examined with incubation in Roswell Park Memorial Institute (RPMI) 1640 medium at 37 °C for up to 6 hours, followed by radio-HPLC analysis.

### B. Cell Culture

HEK293T (ATCC) cells were cultured in Dulbecco's Modified Eagle Medium with 4.5 g/L glucose, 4 mM mg/L L-glutamine, 110 mg/L sodium pyruvate, 10% fetal bovine serum (FBS), 100 U/mL penicillin, and 100 μg/mL streptomycin (all from Gibco). I45 human pleural mesothelioma (Fox Chase Cancer Center, Philadelphia, PA) and SKOV3 human ovarian adenocarcinoma (ATCC) cells were cultured in RPMI 1640 medium with 4.5 g/L glucose, 10 mM HEPES buffer, 2 mM L-glutamine, 1.5 g/L sodium bicarbonate, 110 mg/L sodium pyruvate, 10% FBS, 100 U/mL penicillin, and 100 μg/mL streptomycin (all from Gibco). Cells were maintained in a humidified incubator at 37 °C and 5% CO₂. The cell lines were tested for Mycoplasma using a polymerase chain reaction-based kit (Sigma Aldrich).

C. Expression of Subcellular eDHFR-YFP

eDHFR-YFP gene was inserted into the lentiCRISPR plasmid backbone with restriction enzyme cloning. See, Sellmyer, Journal of the American Society of Gene Therapy. 2020; 28(1):42-51; Shi, Nature biotechnology. 2015; 33(6):661-7. DNA-binding, nuclear, cytoplasmic, and membrane-binding subcellular localization sequences, as detailed in Table 1, were synthesized as gene fragments (Azenta Life Sciences) and were inserted into the lentiviral eDHFR-YFP plasmid with restriction enzyme cloning. See, Kalichuk, Scientific reports. 2016; 6:37274; Lu, Cell communication and signaling: CCS. 2021; 19(1):60; Güttler, Nature structural & molecular biology. 2010; 17(11):1367-76; and Yeung, Science. 2006; 313(5785):347-51. Polyethylenimine (Polysciences #23966) was used to transfect HEK293T cells with the lentiviral plasmids and pVSVg/psPAX2 packaging plasmids. The viral supernatants were filtered through 0.45 μm polyvinylidene fluoride and were administered to I45 and SKOV3 cells in culture media with 8 μg/mL polybrene (Millipore) overnight. After growth in fresh media for 3 days, puromycin was applied (12 μg/mL for I45 and 3 μg/mL for SKOV3) to the growth media for 2 weeks for selection of eDHFR-YFP-positive cells.

### D. Flow Cytometry

For quantification of eDHFR-YFP expression, wild-type and eDHFR-YFP-positive I45 and SKOV3 cells in culture were made into single-cell suspensions using 0.25% trypsin-EDTA (Gibco). The cell suspensions were added to 96-well microplates (Corning #9603) and YFP fluorescence was measured with a Guava EasyCyte High Throughput flow cytometer. FlowJo v10 (Becton, Dickinson and Company) was used for calculation of the YFP fluorescence intensity distribution for each cell type.

### E. Live-Cell Confocal Microscopy

eDHFR-YFP-positive I45 and SKOV3 cells were plated on glass-bottom slides (Greiner #543078) in complete growth media 24 hours before the experiment. One hour prior to imaging, the cellular DNA was stained with Hoechst 33342 (Invitrogen #R37605) and the cells were kept in Hank's Balanced Salt Solution (Gibco) containing 0.33 nM of a fluorescent analog of TMP. Zeiss Laser Scanning Microscope 980 was used with a 100×/1.46 oil immersion objective lens for image acquisition.

F. For quantitative microscopy analysis, the cellular DNA and plasma membrane were stained with Hoechst 33342 and MemBrite® Fix 405/430 (Biotium #30092), respectively, and the cells were subsequently imaged in Hank's Balanced Salt Solution. Z-stack images of individual cells were acquired and exported to Imaris for Core Facilities v10 (Oxford Instruments) for analysis. The Z-stack images were manually segmented for subcellular compartments and background subtraction was applied. The sphericity and volumes of cellular nuclei were measured. The proportion of YFP signal from each subcellular compartment was measured for each cell. In the nuclei of the cells expressing DNA-bound and nuclear eDHFR-YFP, co-localization between Hoechst 33342 and YFP was measured and expressed as Pearson's correlation coefficients. The distance from the cell nucleus was measured for each cytoplasmic and membrane-bound eDHFR-YFP signal.

### G. Radioligand Binding Study

Due to the high specific activity of [²¹¹At]At-TMP that limits its working concentration to pM range, [¹²⁵I]I-TMP was used for saturation binding studies to determine Bₘₐₓ.

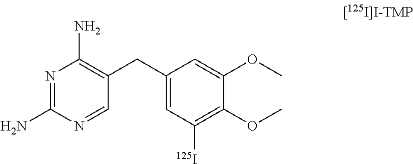 [SMILES: COc1cc(Cc2cnc(N)nc2N)cc([125I])c1OC]

eDHFR-YFP-positive I45 and SKOV3 cells were plated on 96-well plates (Corning #9102) at the density of 25,000 cells/well and 40,000 cells/well, respectively, in complete growth media 24 hours before the assay. The assay was performed with 0.06-60 nM of [¹²⁵I]I-TMP in RPMI 1640±1% bovine serum albumin (BSA), and 10 μM of TMP was used to measure non-specific binding. After incubation at 37 °C for 90 minutes, the wells were aspirated and washed twice with ice-cold PBS. The radioactivity in each well was measured with a Perkin Elmer Wizard gamma counter. The assay was performed three times in triplicates. Since the assay was performed under ligand-depleting conditions due to the low specific activity of [¹²⁵I]I-TMP, the one-site binding model with ligand depletion was used to determine the Bₘₐₓ. See, Swillens, Molecular pharmacology. 1995; 47(6):1197-203. The corresponding EC50 values were plotted against the receptor concentration/2 to determine the K_{d}. See, Hulme, British journal of pharmacology. 2010; 161(6):1219-37. The Bₘₐₓ values were correlated with the total YFP florescence from the wells, with a correction factor of 2.5 and 2.2 for membrane-bound eDHFR-YFP in I45 and SKOV3, respectively, in order to account for decreased fluorescence per molecule from addition of the membrane localization signal to YFP. See, Wu, Science. 2005; 310(5746):310-4; and Bakholdina, Molecules. 2021; 26(13).

For determination of the K_{d} of [²¹¹At]At-TMP, the binding assay protocol was repeated in I45 and SKOV3 cells expressing cytoplasmic eDHFR-YFP, using 150 μL of 0.06-60 nM [¹²⁵I]I-TMP±10 μM TMP for half of each 96-well plate and 50-260 μL of 16 μM [²¹¹At]At-TMP±10 μM TMP for the remaining half. The Bₘₐₓ of each plate determined from [¹²⁵I]I-TMP binding was divided by the assay volumes used for [²¹¹At]At-TMP to calculate the total receptor concentration ([R_{T}]) in each [²¹¹At]At-TMP well. The ligand depletion (δ) in each [²¹¹At]At-TMP well was obtained by dividing the bound radioactivity by total added radioactivity. The assay was performed four times in triplicates. As derived in Supplemental Methods 1, δ⁻¹ was plotted against [R_{T}]⁻¹ to determine the K_{d} as the slope of the linear best-fit line.

### H. Cytotoxicity Assay

Wild-type and eDHFR-YFP-expressing I45 and SKOV3 cells were seeded on 96-well plates (Corning #3603) at the density of 1,000 cells/well and 3,000 cells/well, respectively, in complete growth media 24 hours before the assay. The following five treatment conditions were applied in complete growth media: 200 μCi/mL ~15 μCi/mL [²¹¹At]At-TMP, 120 μCi/mL-15 μCi/mL free ²¹¹At, 120 μCi/mL-15 μCi/mL [²¹¹At]At-TMP+10 μM TMP, 10 nM-1 μM doxorubicin, and 40 μM−1 mM TMP. After 72 hours of incubation, the surviving fraction of the cells was quantified using CellTiter-Glo (Promega #G9243). See, Makvandi, Molecular cancer therapeutics. 2019; 18(7):1195-204. The assay was performed three times in triplicates. The effective concentration for 50% cell death (EC50) was determined for each treatment condition by fitting the surviving fractions into a non-linear sigmoidal dose-response curve. For eDHFR-YFP-positive cells, the [²¹¹At]At-TMP treatment concentrations were converted to the number of ²¹¹At decays/cell, using the median number of receptors/cell (obtained from YFP fluorescence) and K_{d} of [²¹¹At]At-TMP. See, Lee, Journal of nuclear medicine. 2020; 61(6):850-6.

### I. Subcellular Dosimetry

Based on the quantitative microscopy analysis, the nuclei of I45 and SKOV3 cells were modeled as spheres with 6 μm radii. For DNA-bound and nuclear eDHFR-YFP, the nuclear S-values were calculated with MIRDcell v4 (Society of Nuclear Medicine and Molecular Imaging) using a nuclear source of ²¹¹At and daughters. See, Katugampola, MIRD Pamphlet No. 27: MIRDcell V3, a Revised Software Tool for Multicellular Dosimetry and Bioeffect Modeling. Journal of nuclear medicine. 2022; 63(9):1441-9. For cytoplasmic and membrane-bound eDHFR-YFP, the histograms of distance from the cell nucleus (FIG. 26D) were used to calculate the nuclear S-values without assuming a spherical cellular geometry. The nuclear S-values were then expressed relative to the cytoplasmic eDHFR-YFP.

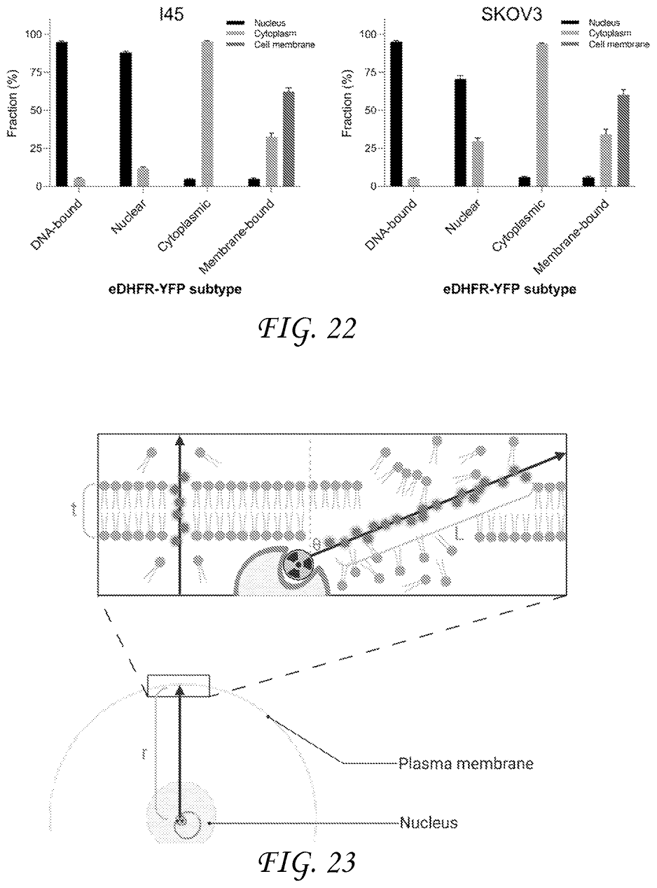

For comparison to the nuclear S-values, the observed cytotoxicity per ²¹¹At decay was also expressed relative to cytoplasmic eDHFR-YFP. Since each eDHFR-YFP subtype did not show 100% localization to the intended subcellular location, the cytotoxicity assay results (FIG. 28C) were combined with the microscopically measured subcellular distribution of each eDHFR-YFP subtype (FIG. 22) to isolate the cytotoxicity of eDHFR-YFP only in the desired subcellular compartment (Supplemental Method 2). Finally, the ratio between the isolated cytotoxicity and nuclear S-value was calculated as the RBE for eDHFR-YFP in each subcellular compartment.

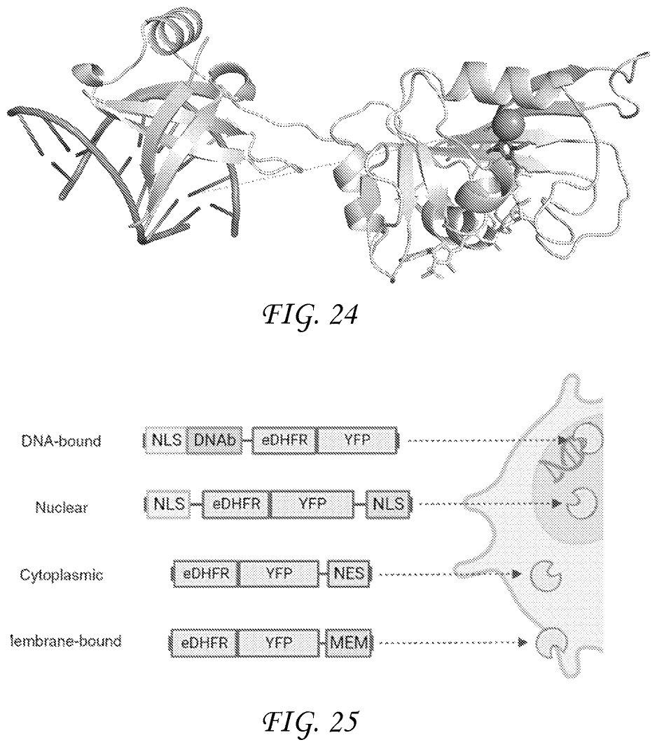

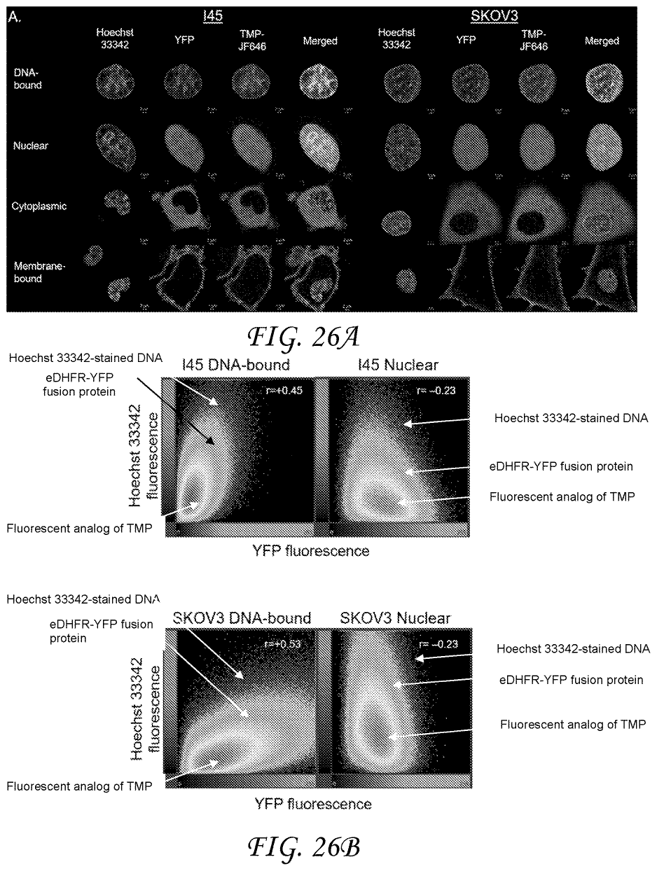

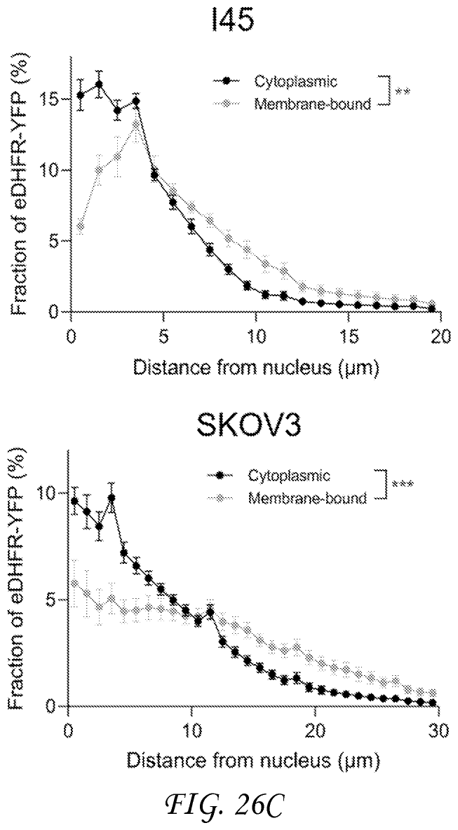

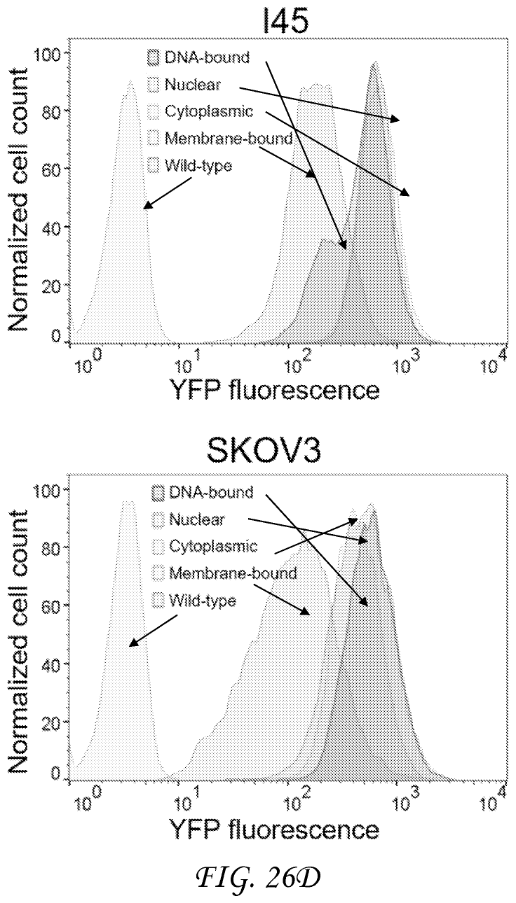

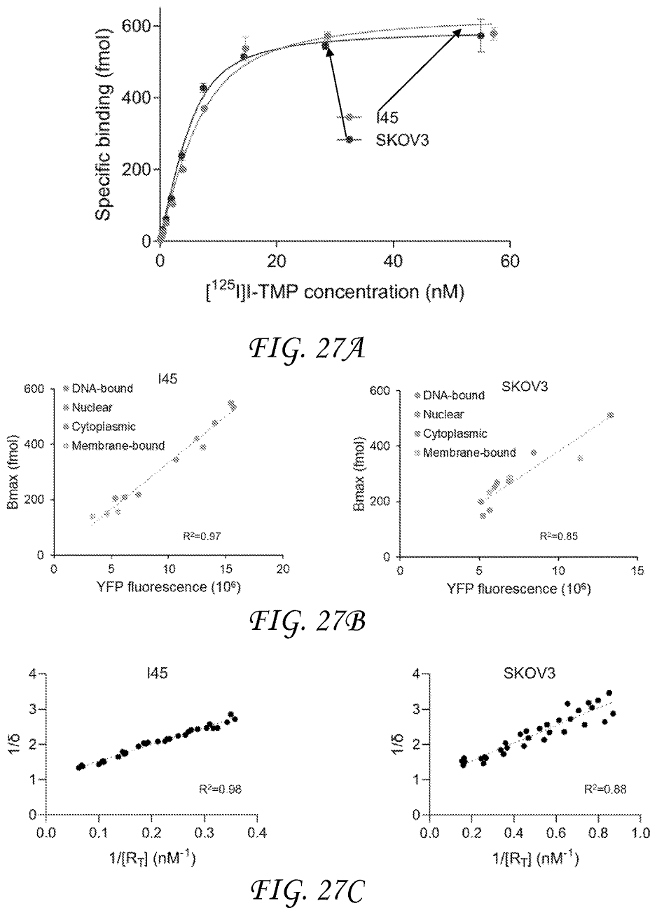

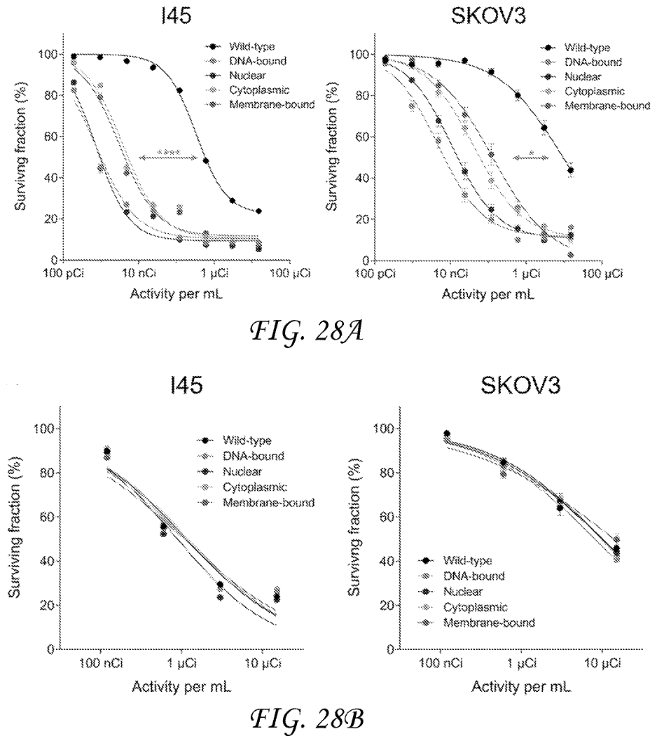

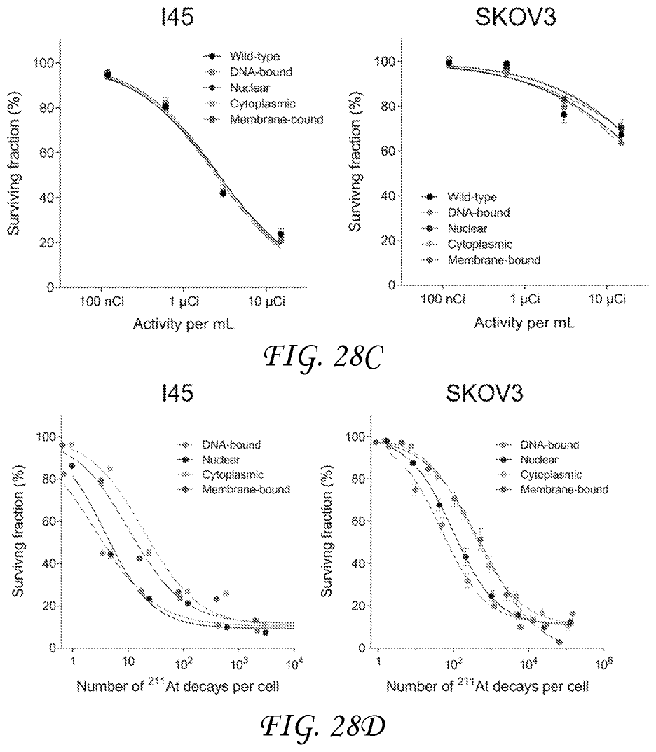

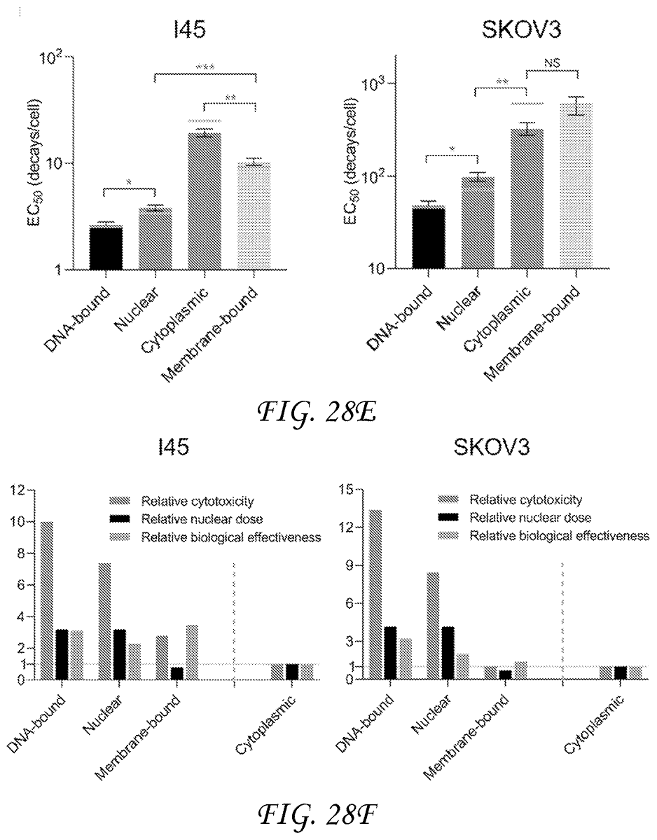

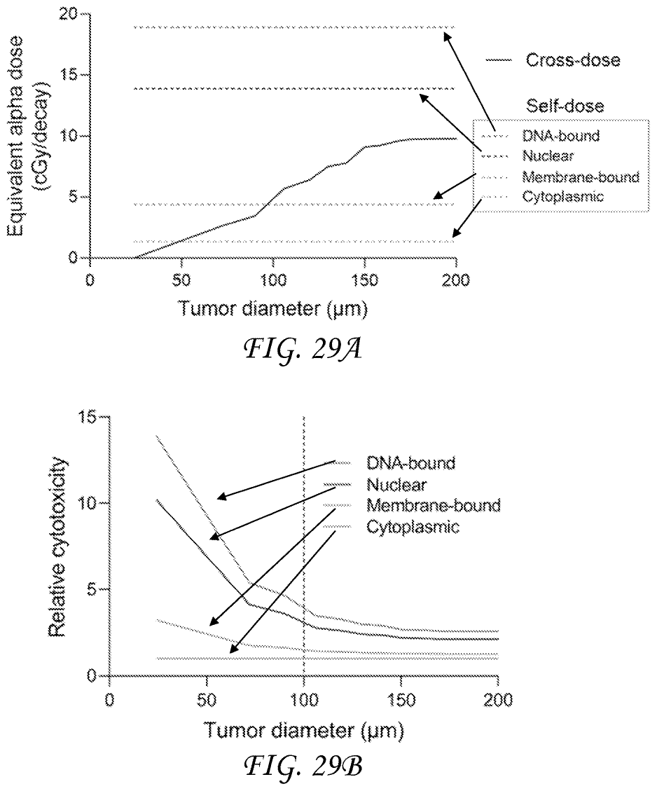

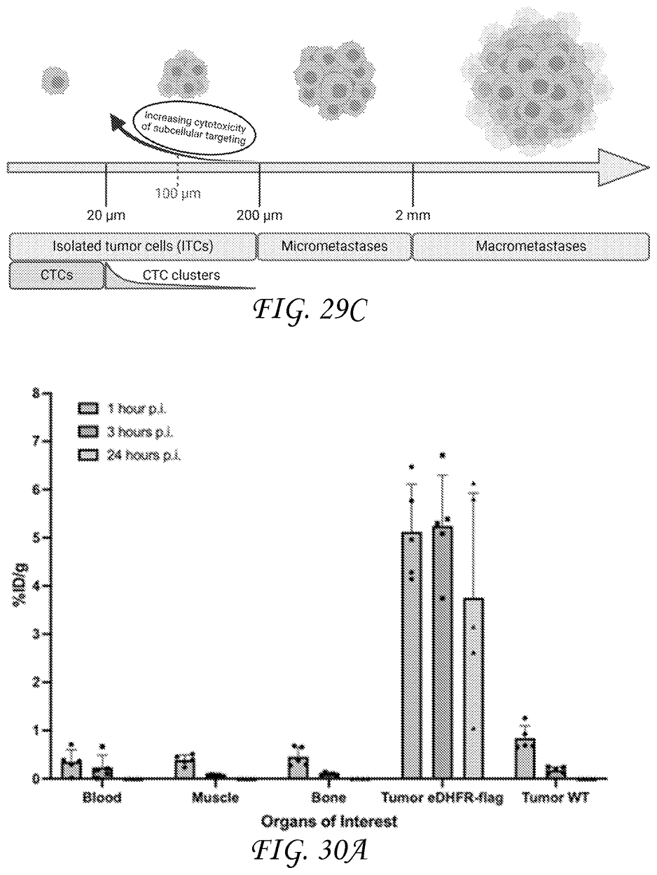

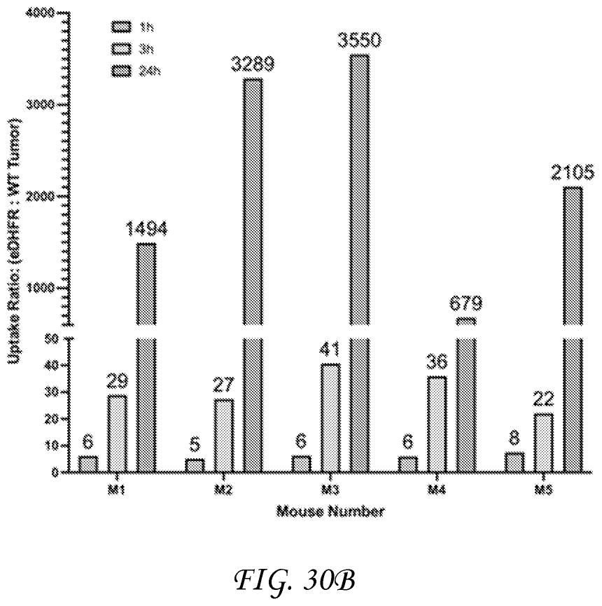

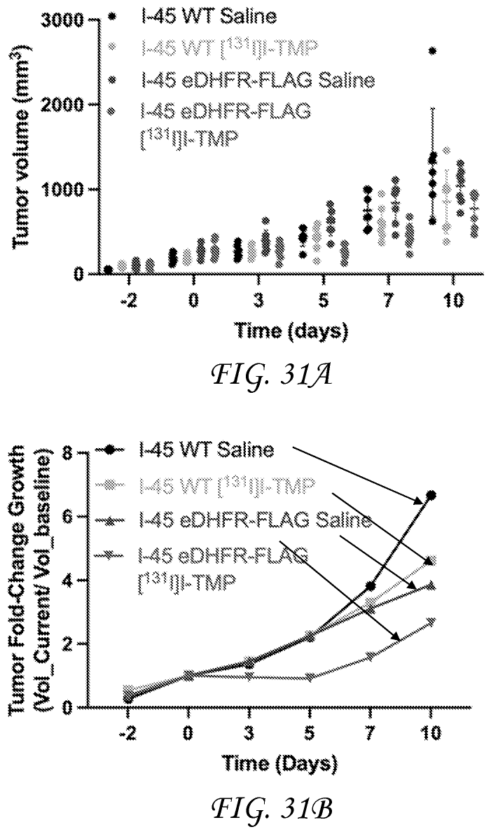

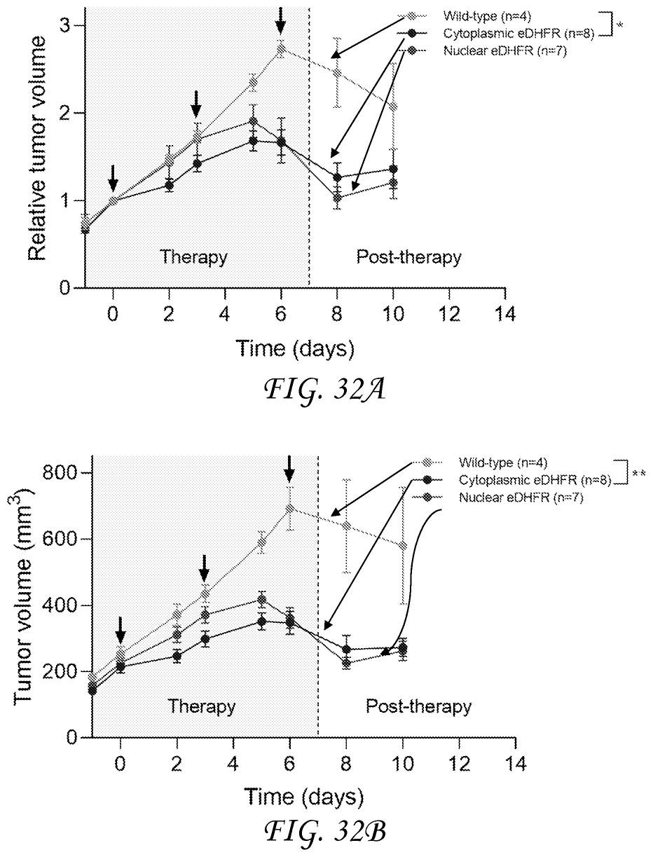

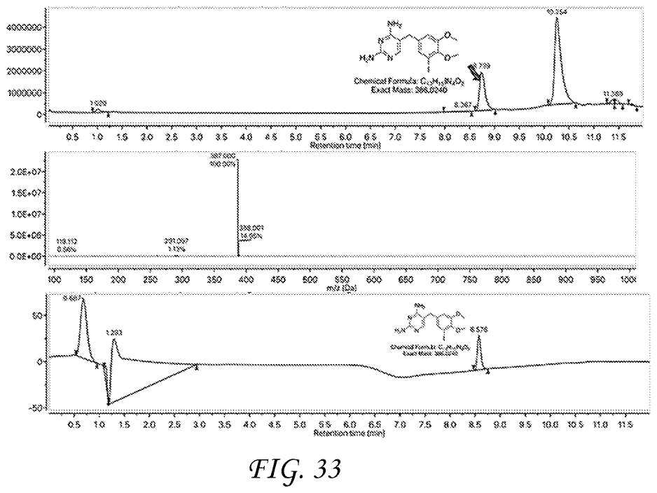

For calculation of the dose to the cell membrane, a cell was modeled as a sphere with 12-μm radius containing a 6-μm radius nucleus. The plasma membrane was modeled as a unit density shell with 8-nm thickness. See, Shan, Chem Soc Rev. 2015; 44(11):3617-38. Then, the average distance within the plasma membrane traversed by an alpha particle originating from the nucleus, cytoplasm, and cytosolic side of the plasma membrane was calculated using the Pythagorean theorem (FIG. 23). The energy deposited to the plasma membrane was then calculated using the average initial LET of 74 keV/μm for alpha particles and the average LET of 1,420 keV/μm for the alpha recoil. See, Kodaira, PLoS One. 2017; 12(6):e0178472; and Roessler, Radiochimica Acta. 1989; 47(2-3):87-90.

For 3-dimensional dosimetry, the spherical cell model was arranged in the highest packing density following the face-centered cubic structure, and the cross-dose from ²¹¹At decay was calculated using the S-values from MIRDcell v4. The equivalent alpha self-dose from the in vitro subcellular dosimetry was then added to calculate the total equivalent alpha dose for ²¹¹At in each subcellular compartment.

### J. Statistical Analysis

Statistical data analysis was performed using Prism v8 (GraphPad). Unpaired, two-tailed Student's t-test was used for evaluation of statistical significance between groups. P values less than 0.05 were considered statistically significant.

Supplemental Method 1. Measurement of the dissociation constant (K_{d}) of [²¹¹At]At-TMP binding to eDHFR under non-saturating, ligand-depleting conditions. When [Ligand]<<K_{d}, ligand depletion (6) at equilibrium is

$$δ = \frac{[ R ]}{{K}_{d} + [ R ]} \approx \frac{[ {R}_{T} ]}{{K}_{d} + [ {R}_{T} ]}$$

where [R] is the free receptor concentration and [R_{T}]is the total receptor concentration. Since 6 is non-zero, taking the reciprocal of both sides of the equation yields

$$\frac{1}{δ} = \frac{{K}_{d} + [ {R}_{T} ]}{[ {R}_{T} ]} = {K}_{d} \cdot \frac{1}{[ {R}_{T} ]} + 1$$

If a δ⁻¹ vs. [R_{T}]⁻¹ graph where y=δ⁻¹ and x=[R_{T}]⁻¹ is plot,

y=K_{d}·x+1

Therefore, the slope of the linear best-fit line represents the K_{d} and the y-intercept is 1.

Supplemental Method 2. Calculation of the relative cytotoxicity of ²¹¹At decay in different subcellular compartments.
By the linear-quadratic model for targeted alpha therapy,

(Survival)=e^{-αD-βD}^{²}≈e^{-αD}
Let N=number of ²¹¹At decays/cell at EC₅₀. Then, the following is obtained:

ln(0.5)=−αD=−TN
where T is the product of S-value and α, representing cytotoxicity per decay. Let F=fraction of eDHFR in each subcellular compartment. Then, the following can be written:

$$[ \begin{matrix}{F}_{d}^{D} & {F}_{d}^{N} & {F}_{d}^{C} & {F}_{d}^{M} \\ {F}_{n}^{D} & {F}_{n}^{N} & {F}_{n}^{C} & {F}_{n}^{M} \\ {F}_{c}^{D} & {F}_{c}^{N} & {F}_{c}^{C} & {F}_{c}^{M} \\ {F}_{m}^{D} & {F}_{m}^{N} & {F}_{m}^{C} & {F}_{m}^{M}\end{matrix} ] \cdot [ \begin{matrix}{T}_{D} \\ {T}_{N} \\ {T}_{C} \\ {T}_{M}\end{matrix} ] = [ \begin{matrix}- ln ( 0.5 ) / {N}_{d} \\ - ln ( 0.5 ) / {N}_{n} \\ - ln ( 0.5 ) / {N}_{c} \\ - ln ( 0.5 ) / {N}_{m}\end{matrix} ]$$

where capital letters denote the microscopically observed subcellular location of eDHFR and lowercase letters denote the genetically targeted location of eDHFR.

The measured values of F (FIG. 19) and N (FIG. 28C) were then used to solve for T. DNA-targeted eDHFR observed in the nucleus is assumed to be 100% DNA-bound, and for SKOV3, N_{c}≈Nₘ is applied based on the lack of significant difference in EC₅₀.

Finally, the T values are normalized with T_{c} to obtain the relative cytotoxicity of ²¹¹At decay in each subcellular compartment compared to the cytoplasm.

TABLE 1
Amino acid sequences for localization of eDHFR-
YFP to specific subcellular locations.

| | | N- or C- | |
|---|---|---|---|
| Location | Sequence name | terminal | Amino acid sequence |
| DNA | Sulfolobus solfataricus | N | MATVKFKYKGEEKEVD |
| | Sso7d DNA-binding | | ISKIKKVWRVGKMISFTY |
| | domain | | DEGGGKTGRGAVSEKD |
| | | | APKELLQMLEKQKK |
| Nucleus | Simian virus 40 large | N | PKKKRKV |
| | tumor antigen nuclear | | |
| | localization signal | | |
| | Nucleoplasmin nuclear | C | KRPAATKKAGQAKKKK |
| | localization signal | | |
| Cytoplasm | Human | C | LPPLERLTL |
| | immunodeficiency virus | | |
| | 1 Rev nuclear export | | |
| | signal | | |
| Cell membrane | KRΦ amphiphilic helix | C | GKKFWKRLRKFLRKLKS |

### K. Results

Astatine-211-astato-trimethoprim ([²¹¹At]At-TMP), an analog of the antibiotic TMP labeled with alpha-emitting ²¹¹At, was developed. Human cancer cells that express Escherichia coli dihydrofolate reductase (eDHFR) were then created, the binding target of TMP, with different subcellular localization domains. Using this model system (FIG. 25), the in vitro cytotoxicity of [²¹¹At]At-TMP in different subcellular compartments was evaluated, and compared the results with subcellular dosimetry.

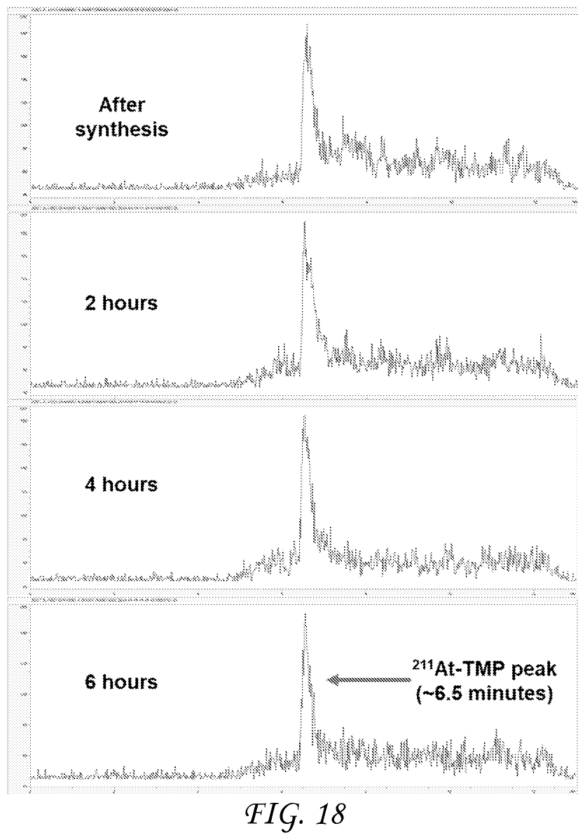

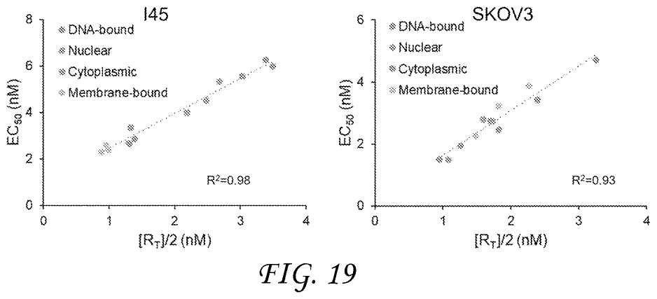

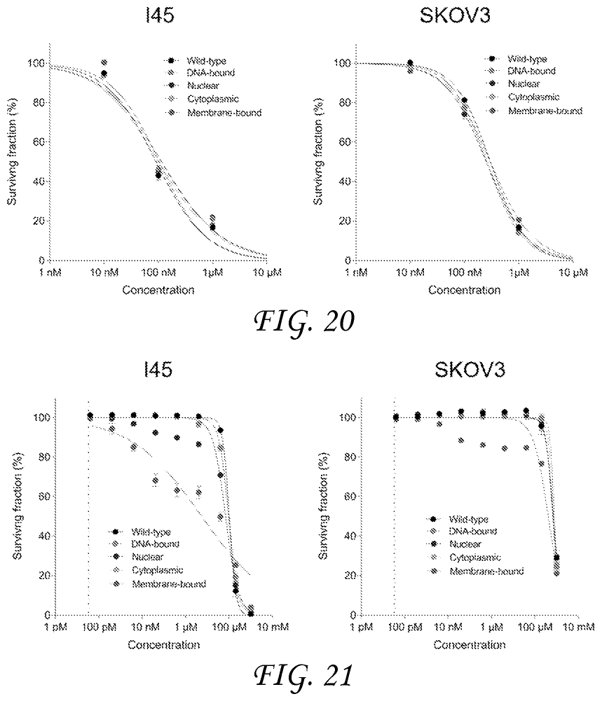

### (i) Radiochemistry

[²¹¹At]At-TMP was successfully isolated at the theoretical specific activity of astatine-211, but [¹²⁵I]I-TMP was isolated at 0.3% of the theoretical specific activity due to its chemical identity to the meta-iodo-TMP precursor. [²¹¹At]At-TMP was found to be stable at 37 °C in aqueous media (FIG. 18).

(ii) Characterization of Subcellular eDHFR-YFP Expression in Human Cancer Cell Lines

Live-cell confocal microscopy in 145 and SKOV3 cells (FIG. 26A) confirmed that the eDHFR-YFP fusion protein was localized to the desired subcellular locations, based on comparison with Hoechst 33342-stained DNA. A fluorescently labeled eDHFR ligand of TMP showed excellent co-localization with eDHFR-YFP, demonstrating successful delivery of TMP to specific subcellular locations.

When co-localization between DNA (Hoechst 33342) and eDHFR (YFP) was measured (FIG. 26B), a significantly positive correlation was found for DNA-bound eDHFR: r=0.43±0.02 in I45 and 0.56±0.01 in SKOV3 (P<0.0001 against zero), verifying subcellular targeting of DNA. In contrast, a significantly negative correlation was observed for nuclear eDHFR: r=−0.10±0.03 in I45 and −0.19±0.02 in SKOV3 (P=0.005 in I45 and P<0.0001 in SKOV3 against zero), suggesting preferential occupancy of DNA-sparse regions in the cell nucleus. The probability histograms of the distance from the cell nucleus (FIG. 26C) show that on average, membrane-bound eDHFR was located approximately 50% further away from the nucleus compared to cytoplasmic eDHFR. Specifically, the average distances for cytoplasmic vs. membrane-bound eDHFR-YFP were 4.2±0.3 μm vs. 6.4±0.5 μm (P=0.0012) in I45 cells and 7.5±0.6 μm vs. 12.2±1.1 μm (P=0.0009) in SKOV3 cells. On flow cytometry (FIG. 26D), YFP fluorescence intensity in eDHFR-YFP positive cells was approximately two orders of magnitude higher than the autofluorescence observed in the wild-type cells.

(iii) Pharmacologic Characterization of Radiolabeled TMP-eDHFR Binding

Saturation binding studies using [¹²⁵I]I-TMP, an iodinated analog of [²¹¹At]At-TMP, yielded the Bₘₐₓ for each eDHFR-YFP subtype (FIG. 27A). The obtained Bₘₐₓ values showed a strongly positive correlation with YFP fluorescence intensity in both I45 (R²=0.97) and SKOV3 (R²=0.85) cell lines (FIG. 27B), supporting the use of YFP fluorescence intensity as a measure of eDHFR-YFP expression.

The [¹²⁵I]I-TMP binding assays yielded a common K_{d} in each cell line across the eDHFR-YFP subtypes (FIG. 19), measured at 0.65±0.11 nM in I45 cells (R²=0.98) and 0.13±0.17 nM in SKOV3 cells (R²=0.92). The K_{d} of [²¹¹At]At-TMP (FIG. 27C) was 4.8±0.1 nM in I45 cells (R²=0.98) and 2.5±0.2 nM in SKOV3 cells (R²=0.88), determined by the slope of the linear best-fit line. The y-intercepts were 1.05 and 1.04, respectively, highly concordant with the theoretical y-intercept of 1 at equilibrium.

(iv) Cytotoxicity of [²¹¹At]at-TMP Based on eDHFR Binding

Compared to the wild-type cells, 2-3 orders of magnitude higher cytotoxicity from [²¹¹At]At-TMP treatment was observed in eDHFR-expressing I45 (P<0.0001) and SKOV3 (P=0.04) cells (FIG. 28A). The difference in cytotoxicity between the wild-type and eDHFR-expressing cells was attributed to specific binding between [²¹¹At]At-TMP and eDHFR, given its disappearance when [²¹¹At]At-TMP was blocked with 10 μM of cold TMP (FIG. 28B). Cytotoxicity of free ²¹¹At (FIG. 28C) was comparable across the wild-type and eDHFR-YFP-expressing cells in both I45 and SKOV3, suggesting that expression of the eDHFR-YFP subtypes did not alter the intrinsic radiosensitivity of the cells. The cells also showed comparable chemosensitivity to doxorubicin (FIG. 20). Interestingly, cells expressing the membrane-bound eDHFR-YFP showed higher cytotoxicity from cold TMP (FIG. 21), attributed to ligand-dependent stabilization of eDHFR-YFP and subsequent membrane disruption from accumulation of the membrane-binding amphiphilic helical domain. Importantly, the lowest TMP concentration of 40 μM was non-toxic in all cell types, suggesting the absence of pharmacologic cytotoxicity from up to 34 μM (dotted line) of [²¹¹At]At-TMP used in the ²¹¹At cytotoxicity assay.

### (v) Quantitative Analysis of [²¹¹At]at-TMP Cytotoxicity Based on Subcellular Localization

When the [²¹¹At]At-TMP concentration was converted to the number of bound [²¹¹At]At-TMP decays/cell (FIG. 28D), the highest cytotoxicity was observed with DNA-bound eDHFR (P=0.017 in I45 and P=0.012 in SKOV3), followed by nuclear eDHFR (P=0.0006 in I45 and P=0.004 in SKOV3) (FIG. 28E). Membrane-bound eDHFR showed higher cytotoxicity compared to cytoplasmic eDHFR in I45 cells (P=0.005), but similar cytotoxicity was observed in SKOV3 cells (P=0.11). When the quantitative fraction of eDHFR-YFP signal from each subcellular compartment (FIG. 22) was used to isolate the cytotoxicity of eDHFR only in the desired subcellular compartment (horizontal bars on FIG. 28E), the following number of ²¹¹At decays per cell was obtained at EC₅₀: 2.5 (DNA-bound), 3.4 (nuclear), 25 (cytoplasmic), and 9.0 (membrane-bound) in I45 cells and 46 (DNA-bound), 72 (nuclear), 610 (cytoplasmic), and 600 (membrane-bound) in SKOV3 cells.

### (vi) Subcellular Dosimetry

The isolated cytotoxicity of eDHFR in each subcellular compartment was then expressed in relative cytotoxicity with reference to cytoplasmic eDHFR (FIG. 28F), yielding the values of 10 (DNA-bound), 7.4 (nuclear), and 2.8 (membrane-bound) in I45 cells and 13 (DNA-bound), 8.5 (nuclear), and 1.0 (membrane-bound) in SKOV3 cells. When the relative cytotoxicity values were divided by the relative nuclear dose per decay (S-values) from subcellular dosimetry (FIG. 28F), the resulting RBE values were 3.1 (DNA-bound), 2.3 (nuclear), and 3.5 (membrane-bound) in I45 cells and 3.2 (DNA-bound), 2.0 (nuclear), and 1.4 (membrane-bound) in SKOV3 cells.

Finally, it was found that an alpha particle emitted from membrane-bound ²¹¹At traverses an approximately 3-times longer segment of the plasma membrane compared to nuclear or cytoplasmic ²¹¹At (FIG. 23). Since this phenomenon is attributed to a higher incident angle between membrane-bound ²¹¹At and the plasma membrane, i.e., the “incident angle effect”. Since the alpha recoil from membrane-bound ²¹¹At also traverses the membrane 50% of the time, the incident angle effect is further amplified by 50%. Taking into account the higher LET of the alpha recoil, the total energy deposited to the plasma membrane by membrane-bound ²¹¹At decay is approximately 30-fold higher than nuclear or cytoplasmic ²¹¹At decay. When the cytotoxicity from plasma membrane dose deposition was compared to nuclear dose deposition, the RBE was 0.11 in I45 and 0.03 in SKOV3. Therefore, the plasma membrane is much less radiosensitive than the nucleus but may represent an important radiobiological target for a membrane-bound alpha emitter.

(vii) Three-Dimensional Dosimetry in Tumor Cell Clusters

Based on 3-dimensional dosimetry, the contribution of cross-dose to the total alpha dose increased progressively with increasing tumor diameter up to −150 μm (FIG. 29A). This cross-dose effect dampened the higher cytotoxicity of DNA-bound, nuclear, and membrane-bound TAT relative to cytoplasmic TAT with increasing tumor size (FIG. 29B). Although DNA-bound and nuclear TAT remained approximately twice as cytotoxic at large tumor sizes, their cytotoxic advantage was maximized below the threshold tumor size of −100 μm.

### L. Discussion

In this study, an alpha-emitting antibiotic [²¹¹At]At-TMP was developed to investigate the differential cytotoxicity of scTAT. First, eDHFR is a completely biologically orthogonal target, and TMP can be considered inert in mammalian cells at low concentrations. Targeting the localized eDHFR with TMP is essentially a forcing function that causes selective accumulation of the radiolabeled alpha-emitting TMP in a specific subcellular locale, which is a unique advantage of this system relative to other PET reporter genes such as sodium iodide symporter or herpes simplex virus type 1 thymidine kinase that cannot provide similar subcellular constraints.

This is the first use of the archaeal nonspecific DNA-binding domain as a tool for DNA localization in living cells. TAT target-drug co-localization in live cells using the eDHFR-YFP fusion protein and a fluorescent analog of TMP was shown.

The cell membrane was redefined as a dosimetry target and the “incident angle effect” was discovered, akin to the solid angle effect for the cell nucleus.

The main finding is that an alpha emitter located in the cell nucleus showed approximately 8-fold higher cytotoxicity per decay compared to a cytoplasmic alpha emitter. While a large part of this higher cytotoxicity is attributed to the solid angle effect, ²¹¹At in the nucleus still showed twice as much cytotoxicity for the same radiation dose to the nucleus (nuclear ²¹¹At vs. cytoplasmic ²¹¹At RBE of 2). This observation subsequently yielded the recoil vs. alpha particle RBE of 3. Alpha recoil is generally not included in subcellular dosimetry, based on an early observation in hamster fibroblasts that found a lower RBE of predominantly nuclear lead-212 compared to predominantly cytoplasmic polonium-210 (4 vs. 6 relative to gamma irradiation). In contrast, early microdosimetry studies predicted that alpha recoil originating in the cell nucleus is an important contributor to cytotoxicity. More recently, ions with LET in the recoil range of ~1,000 keV/μm, compared to the alpha LET range of ~100 keV/μm, were shown to produce more DNA double-strand breaks (DSBs) per Gy and the DSBs were more clustered, which are difficult to repair and therefore highly cytotoxic. A radiobiological concept that seemingly argues against the cytotoxic efficacy of alpha recoil is the “overkill” effect, where increasing LET values above 100 keV/μm leads to decreasing RBE. This effect is attributed to wasted energy deposition by ultra-high-LET particles beyond what is required for cytotoxicity. However, the overkill effect was characterized using external ion beams that traverse the entire cell nucleus to deposit MeV-range energy, and it does not apply to alpha recoil that stops at ~0.1 μm distance to deposit ~100 keV energy. In other words, the ultra-high-LET alpha recoil is efficient at causing many DSBs in clusters at a localized region within the cell nucleus without depositing excess energy, which explains the high RBE of alpha recoil and its contribution to cytotoxicity was observed.

The archaeal DNA-binding domain brings [²¹¹At]At-TMP to the immediate vicinity of DNA at approximately 5-nm distance (FIG. 24), which closely models other radiopharmaceuticals with DNA-binding molecular targets. Interestingly, a 50% higher cytotoxicity from DNA-bound ²¹¹At was observed compared to nuclear ²¹¹At. This phenomenon is attributed to the localization of DNA-bound ²¹¹At to DNA-rich domains within the cell nucleus. Recent advances in microscopy revealed that the density of DNA within the cell nucleus varies by at least 60-fold, from <5 to >300 Mbp/μm³. DNA-bound and free nuclear macromolecules are preferentially found in DNA-rich and DNA-sparse regions, respectively, in agreement with co-localization results. In particular, chromatin packing domains are measured at ~80 nm in radius, and its similarity to the ²¹¹At alpha recoil range of 92 nm suggests highly efficient dose deposition to DNA by the alpha recoil from DNA-bound ²¹¹At. Therefore, results show that even within the cell nucleus, an alpha emitter's cytotoxicity is maximized when its target is associated with DNA.

It was found that membrane-bound ²¹¹At caused similar to 3-fold higher cytotoxicity per decay compared to cytoplasmic ²¹¹At, despite the lower dose deposition to the cell nucleus. Previously, the cell membrane was identified as a more sensitive radiobiological target than cytoplasm for Auger electrons, based on the observation that iodine-125-labeled non-internalizing antibodies caused similar to higher cytotoxicity per decay relative to internalizing antibodies in human cancer cell lines. When the cell membrane is exposed to ionizing radiation, lipid peroxidation results in dose-dependent formation of full-thickness nanopores. The loss of permeability barrier function leads to unregulated diffusion of macromolecules and ions, including influx of calcium ions, which subsequently triggers apoptosis. While this mechanism of cell death was validated also for alpha particles, its degree of contribution to cytotoxicity has been difficult to determine for two main reasons. First, DNA damage-mediated and membrane damage-mediated cytotoxicity by alpha particles share biochemical events such as plasma membrane permeabilization and intracellular calcium accumulation. Second, concurrent nuclear irradiation by alpha particles acts as a confounding factor with high cytotoxicity. Using the scTAT model with subcellular dosimetry, the membrane damage-mediated cytotoxicity was isolated by an alpha emitter. The RBE of 0.03-0.11 for membrane vs. nuclear dose deposition for ²¹¹At is in good agreement with the relative cytotoxicity per decay of approximately 0.05-0.10 from localized irradiation of the cell membrane vs. nucleus by ¹²⁵I.

The 3-dimensional dosimetry results show that while DNA-bound and nuclear alpha emitters are more cytotoxic than cytoplasmic alpha emitters at all tumor sizes, their cytotoxic advantage is maximized below the tumor size threshold of ~100 μm. This size threshold falls within the pathologic definition of “isolated tumor cells” (ITCs), below the size range of “micrometastases” (0.2-2 mm) (FIG. 29C). Hematologically, solid tumor cells may be seen as individual circulating tumor cells (CTCs) or CTC clusters. Most CTCs circulate as singlets or doublets, and clustered CTCs are generally smaller than 100 μm. Therefore, results suggest that DNA-bound and nuclear alpha emitters are particularly suited for treatment of ITCs, CTCs, and CTC clusters.

Compared to [¹²⁵I]I-TMP, a lower binding affinity for [²¹¹At]At-TMP with K_{d} of 2.5-4.8 nM was found. Nevertheless, results show that [²¹¹At]At-TMP is still a high-affinity ligand with a nanomolar-range K_{d} and selectively for eDHFR.

Investigating the sensitivity to scTAT in normal tissue models of dose-limiting organs would help understand the therapeutic benefit of scTAT for clinical translation. In addition to the subcellular locations investigated, various intracellular localization domains can be used to determine the radiosensitivity of previously unexplored subcellular targets, such as the mitochondria or nuclear envelope. Beyond scTAT, ²¹¹At-labeled TMP may be used in the future as a cytotoxic drug for CAR T-cells or tumor cells with eDHFR expression as a suicide gene. Compared to other suicide gene systems such as inducible caspase 9, unique features of this approach are the potential to deliver alpha particle irradiation to adjacent tumor cells without eDHFR expression, as well as the availability of [¹⁸F]fluoropropyl-TMP as an imaging theranostic pair to verify tumor targeting.

In conclusion, it was found that an alpha emitter's proximity to DNA leads to higher cytotoxicity, and the plasma membrane may also represent a radiobiological target. The alpha recoil was a significant contributor to the cytotoxicity of nuclear and DNA-bound alpha emission, and the incident angle effect was defined that explains the cytotoxicity of membrane-bound alpha emission. The cytotoxic advantage of DNA-bound and nuclear TAT was maximized in ITCs, CTCs, and CTC clusters.

The disclosures of each patent, patent application, and publication cited or described in this document are hereby incorporated herein by reference, each in its entirety, for all purposes.

1. A compound of formula I, II, or III: 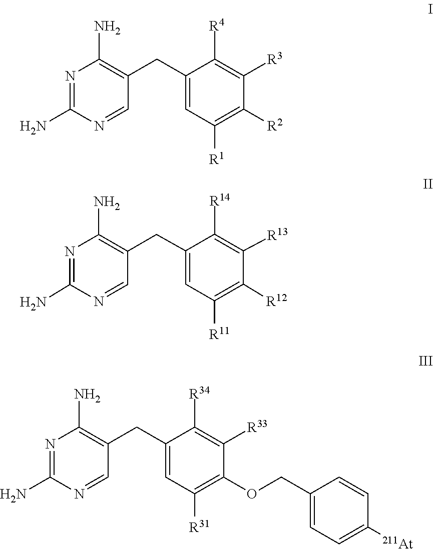
wherein:
R¹, R³, and R⁴ are, independently, H or radioactive halo;
R² is H, halo, C₁₋₆alkoxy, C₁₋₆alkyl, or aryl, provided that at least one of R¹, R3, and R⁴ is radioactive halo;
R¹² is a radioactive halo;
R¹¹, R¹³, and R¹⁴ are, independently, H, C₁₋₆alkoxy, C₁₋₆alkyl, or aryl, provided that at least one of R¹¹, R¹³, and R¹⁴ is C₁₋₆alkyl or aryl; and
R³¹, R³³, and R³⁴ are, independently, H, C₁₋₆alkoxy, C₁₋₆alkyl, or aryl;
or a pharmaceutically acceptable salt thereof.

2. (canceled)

3. The compound of claim 1, wherein R¹, R³, R⁴, and/or R¹² is ¹²⁵I.

4. The compound of claim 1, wherein R¹, R³, R⁴, and/or R¹² is ¹³¹I.

5. The compound of claim 1, wherein R¹, R³, R⁴, and/or R¹² is ²¹¹At.

6. The compound of claim 1, wherein R¹, R³, R⁴, and/or R¹² is ⁷⁵Br.

7. The compound of claim 1, wherein R¹, R³, R⁴, and/or R¹² is ⁷⁶Br.

8. The compound of claim 1, wherein R¹, R³, R⁴, and/or R¹² is ⁷⁷Br.

9. The compound of claim 1, wherein R² or one or more of R¹¹, R¹³, R¹⁴, R³¹, R³³, and/or R³⁴ is H.

10. The compound of claim 1, wherein R² or one or more of R¹¹, R¹³, R¹⁴, R³¹, R³³, and/or R³⁴ is C₁₋₆alkoxy.

11. The compound of claim 1, wherein R² or one or more of R¹¹, R¹³, R¹⁴, R³¹, R³³, and/or R³⁴ is C₁₋₆alkyl.

12. The compound of claim 1, wherein R² or one or more of R¹¹, R¹³, R¹⁴, R³¹, R³³, and/or R³⁴ is aryl.

13. The compound of claim 1, wherein R² is halo.

14. The compound of claim 1, that is of formula I-A or I-B:

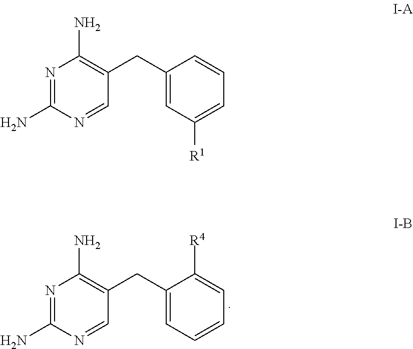

15. The compound of claim 1 that is 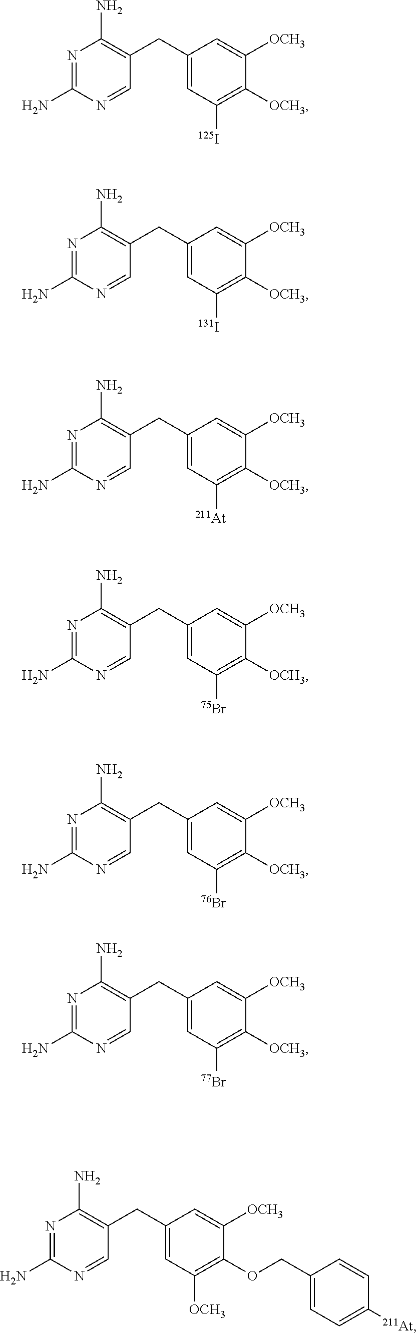
or a pharmaceutically acceptable salt thereof.

16. (canceled)

17. A composition comprising one or more compounds of claim 1 and a pharmaceutically acceptable carrier or diluent.

18. A method of delivering radiation to dihydrofolate reductase (DHFR) expressing cells or tissues in a subject, comprising administering the compound of claim 1 to the DHFR expressing cells or tissues.

19. A method of killing a cell or tissue comprising E. coli dihydrofolate reductase (eDHFR), comprising exposing the cell or tissue to the compound of claim 1.

20. A method of treating a cancer in a subject in need thereof, comprising:
(a) delivering eDHFR to cells or tissue surrounding the cancer; and
(b) administering an effective amount of the compound of claim 1 to said subject.

21-31. (canceled)

32. A method of engineering a radioligand binding domain to target a nucleus or DNA of a cell, comprising:
(i) fusing a nucleic acid encoding the radioligand binding domain to a nucleic acid encoding a tag that spatially constrains the location of the radioligand binding domain to a specific subcellular locale to provide a fusion genetic construct; and
(ii) expressing one or more proteins of the fusion genetic construct in the cell.

33-39. (canceled)
